(19) United States
(12) Patent Application Publication
(10) Pub. No.: US 2023/0089530 A1
(43) Pub. Date: Mar. 23, 2023

(54) QUINUCLIDINONE ANALOGUES AS ANTICANCER AGENTS

(71) Applicant: Newave Pharmaceutical Inc., Pleasanton, CA (US)

(72) Inventor: Yi Chen, Pleasanton, CA (US)

(21) Appl. No.: 17/797,772

(22) PCT Filed: Feb. 5, 2021
(86) PCT No.: PCT/US2021/016862

§ 371 Date: Aug. 5, 2022

Related U.S. Application Data
(60) Provisional application No. 63/053,592, filed on Jul. 18, 2020.
(60) Provisional application No. 63/019,374, filed on May 3, 2020.
(60) Provisional application No. 63/002,106, filed on Mar. 30, 2020.
(60) Provisional application No. 62/972,002, filed on Feb. 9, 2020.

(51) Int. Cl. C07D 453/02; C07F 9/6561; A61P 35/02; A61P 35/00; A61K 9/00

(52) U.S. Cl. CPC C07D 453/02; C07F 9/6561; A61P 35/02; A61P 35/00; A61K 9/0053; C07B 2200/07

(57) ABSTRACT

The disclosure includes compounds of Formula (I) and Formula (A) wherein R₁, R₂, R₃, m, n, k, and L are defined herein. Also disclosed are methods for treating a neoplastic disease, autoimmune disease, or an inflammatory disorder with these compounds.

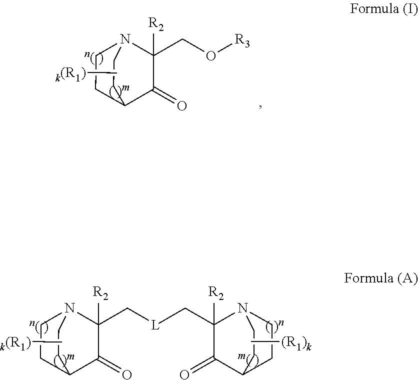

### REFERENCE TO RELATED APPLICATIONS

This application claims the benefit of the filing date of U.S. Provisional Patent Application Nos. 62/972,002, filed on Feb. 9, 2020; 63/002,106, filed on Mar. 30, 2020; 63/019,374, filed on May 3, 2020; and 63/053,592, filed on Jul. 18, 2020 the entire contents of each of the above-referenced applications are incorporated herein by reference.

### BACKGROUND OF THE INVENTION

APR-246 and PRIMA-1 have been reported to have anticancer activities [Bykov V J, et al, Nature Medicine. 2002 March; 8(3):282-8, Lambert J M, et al, Cancer Cell. 2009 May 5, 15(5), 376-88; Perdrix A, et al, Cancers (Basel). 2017 Dec. 16, 9(12); Zhang Q, Cell Death Dis. 2018 May 1, 9(5), 439; Omar S I, et al, Oncotarget. 2018 Dec. 14; 9(98):37137-37156].

APR-246 is in the Phase III trial for cancer patients. One major disadvantage of APR-246 is that it is an intravenous drug. The injection must be delivered in a clinic which limits access to many patients in remote areas, stresses the patients and their caregivers, and adds cost to the health care system.

Although APR-246 and PRIMA-1, have made a significant contribution to the art, there is a strong need for continuing search in this field of art for improved pharmaceuticals with acceptable oral bioavailability.

### SUMMARY OF THE INVENTION

The present invention relates to a class of derivatives of quinuclidin-3-one. Thus, the compounds of the present invention may be useful in treating the cancer patient. The compounds of the present invention may be useful in treating the patients with diseases such as autoimmune disease, or inflammatory disorders.

In one aspect, this invention relates to a compound of Formula (I), or an N-oxide thereof, or a pharmaceutically acceptable salt, solvate, polymorph, tautomer, stereoisomer, an isotopic form, or a prodrug of said compound of Formula (I) or N-oxide thereof:

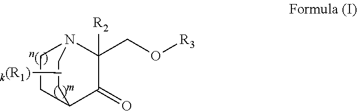

wherein
k is 0, 1, 2, 3, 4, 5, or 6;
R₁ is H, alkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, alkenyl, alkynyl, cycloalkyl, cycloalkenyl, heterocycloalkyl, spiroheterocycloalkyl, heterocycloalkenyl, aryl, heteroaryl is optionally substituted with one or more R_{d};
R₂ is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, alkyl-ORₐ, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, —S(O)(═N(R_{b}))R_{c}, —N═S(O)R_{b}R_{c}, ═NR_{b}, —SO₂N(R_{b})R_{c}, —N(R_{b})SO₂R_{c}, in which said cycloalkyl, cycloalkenyl, heterocycloalkyl, heterocycloalkenyl, aryl, heteroaryl is optionally substituted with one or more R_{d};
R₃ is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, —S(O)(═N(R_{b}))R_{c}, —N═S(O)R_{b}R_{c}, ═NR_{b}, —SO₂N(R_{b})R_{c}, —N(R_{b})SO₂R_{c},

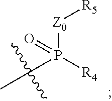

in which said cycloalkyl, cycloalkenyl, heterocycloalkyl, heterocycloalkenyl, aryl, heteroaryl is optionally substituted with one or more R_{d};
Z₀ is absent, O, N(Rₐ), or S;
R₄ is alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH—(CHR_{b})COOR_{c}, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
R₅ is alkyl, spiroalkyl, alkenyl, alkynyl, cycloalkyl, cycloalkenyl, heterocycloalkyl, spiroheterocycloalkyl, heterocycloalkenyl, aryl, heteroaryl, -alkyl-Rₐ, —NH(CH₂)ₚRₐ, —C(O)Rₐ, —S(O)Rₐ, —SO₂Rₐ, —C(O)ORₐ, —C(O)ORₐ, -alkyl-OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, C(O)NHOH, C(O)OH, —C(O)O-alkyl, —C(O)O-aryl, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ; and
Rₑ is H, D, alkyl, spiroalkyl, alkenyl, alkynyl, halo, cyano, amine, nitro, hydroxy, ═O, C(O)NHOH, —C(O)O-alkyl, —C(O)O-aryl, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl.
two of R₁ groups, taken together with the atom to which they are attached, may optionally form a cycloalkyl or heterocycloalkyl optionally substituted with one or more R_{d};
R₂ and R₃ groups, taken together with the atom to which they are attached, may optionally form a cycloalkyl or heterocycloalkyl optionally substituted with one or more R_{d}; and
each of m, n, k, and p, independently, is 0, 1, 2, or 3.

In some embodiments, the compound is represented by Formula (II)

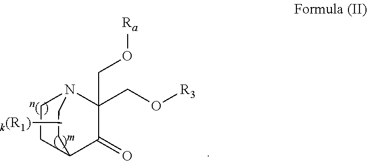

In some embodiments, the compound is represented by Formula (III)

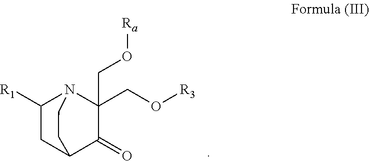

In some embodiments, the compound is represented by Formula (V)

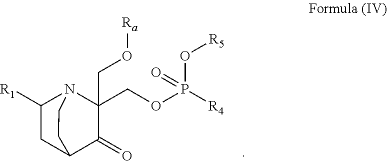

In some embodiments, the compound is represented by Formula (IV)

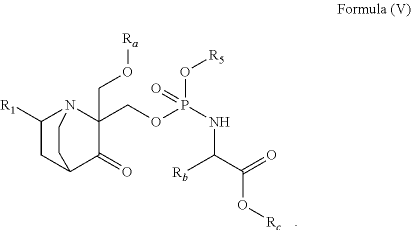

In another aspect, this invention relates to a compound of Formula (A), or an N-oxide thereof, or a pharmaceutically acceptable salt, solvate, polymorph, tautomer, stereoisomer, an isotopic form, or a prodrug of said compound of Formula (A) or N-oxide thereof:

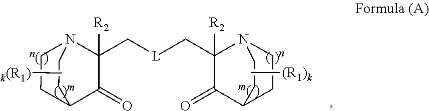

wherein
k is 0, 1, 2, 3, 4, 5, or 6;
R₁ is H, alkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, alkenyl, alkynyl, cycloalkyl, cycloalkenyl, heterocycloalkyl, spiroheterocycloalkyl, heterocycloalkenyl, aryl, heteroaryl is optionally substituted with one or more R_{d};
R₂ is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, alkyl-ORₐ, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, —S(O)(═N(R_{b}))R_{c}, —N═S(O)R_{b}R_{c}, —NR_{b}, —SO₂N(R_{b})R_{c}, —N(R_{b})SO₂R_{c}, in which said cycloalkyl, cycloalkenyl, heterocycloalkyl, heterocycloalkenyl, aryl, heteroaryl is optionally substituted with one or more R_{d};
L is -L₁-L₂-L₃-L₄-L₅;
each of L₁, L₂, L₃, L₄, and L₅, independently, is absent, —O—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—,

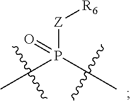

a bivalent alkyl group, a bivalent alkenyl group, a bivalent alkynyl group, a bivalent cycloalkyl group, a bivalent heterocycloalkyl group, a bivalent aryl group, a bivalent heteroaryl group, in which said bivalent groups are optionally substituted with one or more R_{d},
Z is absent, O, or N(Rₐ);
R₆ is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —(CHR_{b})COOR_{c}, —C(O)Rₐ, —S(O)Rₐ, —SO₂Rₐ, —C(O)ORₐ, —OC(O)Rₐ, —NHR_{b}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, C(O)NHOH, C(O)OH, —C(O)O-alkyl, —OC(O)-alkyl, —C(O)O-aryl, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ; and
Rₑ is H, D, alkyl, spiroalkyl, alkenyl, alkynyl, halo, cyano, amine, nitro, hydroxy, ═O, C(O)NHOH, —OC(O)-alkyl, —C(O)O-alkyl, —C(O)O-aryl, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more D, alkyl, spiroalkyl, alkenyl, alkynyl, halo, cyano, amine, nitro, hydroxy, ═O, C(O)NHOH, —OC(O)-alkyl, —C(O)O-alkyl, —C(O)O-aryl, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl,
heterocycloalkyl, spiroheterocycloalkyl, heterocycloalkenyl, aryl, or heteroaryl; each of m, n, k, and p, independently, is 0, 1, 2, or 3.

In some embodiments, the compound is represented by Formula (B)

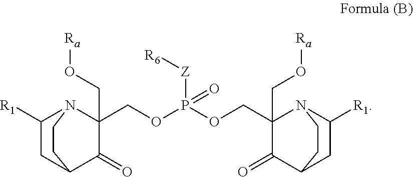

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above for use in treating a neoplastic disease, autoimmune disease, and inflammatory disorders, therapeutic uses thereof, and use of the compounds for the manufacture of a medicament for treating the disease/disorder.

This invention also relates to a method of treating a neoplastic disease, by administering to a subject in need thereof an effective amount of one or more of the compounds, modifications, and/or salts, and compositions thereof described above.

In some examples, the neoplastic disease is characterized by a mutant p53. In some examples, the compound of Formula (I) or (B); the N-oxide thereof; or the pharmaceutically acceptable salt, solvate, polymorph, tautomer, stereoisomer, isotopic form, or prodrug thereof, restores biological function to the mutant p53. In other examples, the neoplastic disease is characterized by inactivated p53.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:

List 1
(1s,4s)-2,2-bis(hydroxymethyl)-4-methylquinuclidin-3-one,
(1R,2S,4R)-2-(hydroxymethyl)-2-(methoxymethyl)-4-methylquinuclidin-3-one,
(1S,2R,4S)-2-(hydroxymethyl)-2-(methoxymethyl)-4-methylquinuclidin-3-one,
(1S,2S,4S)-4-fluoro-2-(hydroxymethyl)-2-(methoxymethyl)quinuclidin-3-one,
(1R,2R,4R)-4-fluoro-2-(hydroxymethyl)-2-(methoxymethyl)quinuclidin-3-one,
(1S,2S,4S)-2-(hydroxymethyl)-2-(methoxymethyl)-4-(trifluoromethyl)quinuclidin-3-one,
(1R,2R,4R)-2-(hydroxymethyl)-2-(methoxymethyl)-4-(trifluoromethyl)quinuclidin-3-one,
(1R,2S,4R)-2-(hydroxymethyl)-4-isopropyl-2-(methoxymethyl)quinuclidin-3-one,
(1S,2R,4S)-2-(hydroxymethyl)-4-isopropyl-2-(methoxymethyl)quinuclidin-3-one,
(1R,2S,4R)-4-ethyl-2-(hydroxymethyl)-2-(methoxymethyl)quinuclidin-3-one,
(1S,2R,4S)-4-ethyl-2-(hydroxymethyl)-2-(methoxymethyl)quinuclidin-3-one,
(1R,2S,4R)-4-(tert-butyl)-2-(hydroxymethyl)-2-(methoxymethyl)quinuclidin-3-one,
(1S,2R,4S)-4-(tert-butyl)-2-(hydroxymethyl)-2-(methoxymethyl)quinuclidin-3-one,
(1R,2S,4R)-2-(hydroxymethyl)-2-(methoxymethyl)-4-(1,1,1-trifluoro-2-methylpropan-2-yl)quinuclidin-3-one,
(1S,2R,4S)-2-(hydroxymethyl)-2-(methoxymethyl)-4-(1,1,1-trifluoro-2-methylpropan-2-yl)quinuclidin-3-one,
(1R,2S,4R)-4-cyclopropyl-2-(hydroxymethyl)-2-(methoxymethyl)quinuclidin-3-one,
(1S,2R,4S)-4-cyclopropyl-2-(hydroxymethyl)-2-(methoxymethyl)quinuclidin-3-one,
(1R,2S,4R)-4-cyclobutyl-2-(hydroxymethyl)-2-(methoxymethyl)quinuclidin-3-one,
(1S,2R,4S)-4-cyclobutyl-2-(hydroxymethyl)-2-(methoxymethyl)quinuclidin-3-one,
(1R,2S,4R)-4-cyclopentyl-2-(hydroxymethyl)-2-(methoxymethyl)quinuclidin-3-one,
(1S,2R,4S)-4-cyclopentyl-2-(hydroxymethyl)-2-(methoxymethyl)quinuclidin-3-one,
(1R,2S,4R)-4-cyclohexyl-2-(hydroxymethyl)-2-(methoxymethyl)quinuclidin-3-one,
(1S,2R,4S)-4-cyclohexyl-2-(hydroxymethyl)-2-(methoxymethyl)quinuclidin-3-one,
(1R,2S,4R)-2-(hydroxymethyl)-2-(methoxymethyl)-4-phenylquinuclidin-3-one,
(1S,2R,4S)-2-(hydroxymethyl)-2-(methoxymethyl)-4-phenylquinuclidin-3-one,
(1R,2S,4R)-2-(hydroxymethyl)-2-(methoxymethyl)-4-(pyridin-4-yl)quinuclidin-3-one,
(1S,2R,4S)-2-(hydroxymethyl)-2-(methoxymethyl)-4-(pyridin-4-yl)quinuclidin-3-one,
(1R,2S,4R)-2-(hydroxymethyl)-2-(methoxymethyl)-4-(pyridin-2-yl)quinuclidin-3-one,
(1R,2S,4R)-2-(hydroxymethyl)-2-(methoxymethyl)-4-(pyridin-3-yl)quinuclidin-3-one,
(1S,2R,4S)-2-(hydroxymethyl)-2-(methoxymethyl)-4-(pyridin-2-yl)quinuclidin-3-one,
(1S,2R,4S)-2-(hydroxymethyl)-2-(methoxymethyl)-4-(pyridin-3-yl)quinuclidin-3-one,
(1R,2S,4R)-5,5-difluoro-2-(hydroxymethyl)-2-(methoxymethyl)-4-methylquinuclidin-3-one,
(1S,2S,4S)-5,5-difluoro-2-(hydroxymethyl)-2-(methoxymethyl)-4-methylquinuclidin-3-one,
(1R,2R,4R)-5,5-difluoro-2-(hydroxymethyl)-2-(methoxymethyl)-4-methylquinuclidin-3-one,
(1S,2R,4S)-5,5-difluoro-2-(hydroxymethyl)-2-(methoxymethyl)-4-methylquinuclidin-3-one,
(1R,2S,4S)-2-(hydroxymethyl)-2-(methoxymethyl)-4,5,5-trimethylquinuclidin-3-one,
(1S,2S,4R)-2-(hydroxymethyl)-2-(methoxymethyl)-4,5,5-trimethylquinuclidin-3-one,
(1R,2R,4S)-2-(hydroxymethyl)-2-(methoxymethyl)-4,5,5-trimethylquinuclidin-3-one,
(1S,2R,4R)-2-(hydroxymethyl)-2-(methoxymethyl)-4,5,5-trimethylquinuclidin-3-one,
(1R,2S,4S)-2-(hydroxymethyl)-2-(methoxymethyl)-4,6,6-trimethylquinuclidin-3-one,
(1S,2S,4R)-2-(hydroxymethyl)-2-(methoxymethyl)-4,6,6-trimethylquinuclidin-3-one,
(1R,2R,4S)-2-(hydroxymethyl)-2-(methoxymethyl)-4,6,6-trimethylquinuclidin-3-one,
(1S,2R,4R)-2-(hydroxymethyl)-2-(methoxymethyl)-4,6,6-trimethylquinuclidin-3-one,
(1R,2S,4S,6S)-2-(hydroxymethyl)-2-(methoxymethyl)-4,6-dimethylquinuclidin-3-one,
(1S,2S,4R,6S)-2-(hydroxymethyl)-2-(methoxymethyl)-4,6-dimethylquinuclidin-3-one,
(1R,2R,4S,6S)-2-(hydroxymethyl)-2-(methoxymethyl)-4,6-dimethylquinuclidin-3-one,
(1S,2R,4R,6S)-2-(hydroxymethyl)-2-(methoxymethyl)-4,6-dimethylquinuclidin-3-one,
(1R,2S,4S,6R)-2-(hydroxymethyl)-2-(methoxymethyl)-4,6-dimethylquinuclidin-3-one,
(1S,2S,4R,6R)-2-(hydroxymethyl)-2-(methoxymethyl)-4,6-dimethylquinuclidin-3-one,
(1R,2R,4S,6R)-2-(hydroxymethyl)-2-(methoxymethyl)-4,6-dimethylquinuclidin-3-one,
(1S,2R,4R,6R)-2-(hydroxymethyl)-2-(methoxymethyl)-4,6-dimethylquinuclidin-3-one,
(1R,2S,4S,6R)-2-(hydroxymethyl)-2-(methoxymethyl)-4-methyl-6-(trifluoromethyl)quinuclidin-3-one,
(1S,2S,4R,6R)-2-(hydroxymethyl)-2-(methoxymethyl)-4-methyl-6-(trifluoromethyl)quinuclidin-3-one,
(1R,2R,4S,6R)-2-(hydroxymethyl)-2-(methoxymethyl)-4-methyl-6-(trifluoromethyl)quinuclidin-3-one,
(1S,2R,4R,6R)-2-(hydroxymethyl)-2-(methoxymethyl)-4-methyl-6-(trifluoromethyl)quinuclidin-3-one,
(1R,2S,4S,6S)-2-(hydroxymethyl)-2-(methoxymethyl)-4-methyl-6-(trifluoromethyl)quinuclidin-3-one,
(1S,2S,4R,6S)-2-(hydroxymethyl)-2-(methoxymethyl)-4-methyl-6-(trifluoromethyl)quinuclidin-3-one,
(1R,2R,4S,6S)-2-(hydroxymethyl)-2-(methoxymethyl)-4-methyl-6-(trifluoromethyl)quinuclidin-3-one,
(1S,2R,4R,6S)-2-(hydroxymethyl)-2-(methoxymethyl)-4-methyl-6-(trifluoromethyl)quinuclidin-3-one,
(1R,2S,4S,5S)-2-(hydroxymethyl)-2-(methoxymethyl)-4,5-dimethylquinuclidin-3-one,
(1S,2S,4R,5S)-2-(hydroxymethyl)-2-(methoxymethyl)-4,5-dimethylquinuclidin-3-one,
(1R,2R,4S,5S)-2-(hydroxymethyl)-2-(methoxymethyl)-4,5-dimethylquinuclidin-3-one,
(1S,2R,4R,5S)-2-(hydroxymethyl)-2-(methoxymethyl)-4,5-dimethylquinuclidin-3-one,
(1R,2S,4S,5R)-2-(hydroxymethyl)-2-(methoxymethyl)-4,5-dimethylquinuclidin-3-one,
(1S,2S,4R,5R)-2-(hydroxymethyl)-2-(methoxymethyl)-4,5-dimethylquinuclidin-3-one,
(1R,2R,4S,5R)-2-(hydroxymethyl)-2-(methoxymethyl)-4,5-dimethylquinuclidin-3-one,
(1S,2R,4R,5R)-2-(hydroxymethyl)-2-(methoxymethyl)-4,5-dimethylquinuclidin-3-one,
(1R,2S,4S,5S)-2-(hydroxymethyl)-2-(methoxymethyl)-4-methyl-5-(trifluoromethyl)quinuclidin-3-one,
(1S,2S,4R,5S)-2-(hydroxymethyl)-2-(methoxymethyl)-4-methyl-5-(trifluoromethyl)quinuclidin-3-one,
(1R,2R,4S,5S)-2-(hydroxymethyl)-2-(methoxymethyl)-4-methyl-5-(trifluoromethyl)quinuclidin-3-one,
(1S,2R,4R,5S)-2-(hydroxymethyl)-2-(methoxymethyl)-4-methyl-5-(trifluoromethyl)quinuclidin-3-one,
(1R,2S,4S,5R)-2-(hydroxymethyl)-2-(methoxymethyl)-4-methyl-5-(trifluoromethyl)quinuclidin-3-one,
(1S,2S,4R,5R)-2-(hydroxymethyl)-2-(methoxymethyl)-4-methyl-5-(trifluoromethyl)quinuclidin-3-one,
(1R,2R,4S,5R)-2-(hydroxymethyl)-2-(methoxymethyl)-4-methyl-5-(trifluoromethyl)quinuclidin-3-one,
(1S,2R,4R,5R)-2-(hydroxymethyl)-2-(methoxymethyl)-4-methyl-5-(trifluoromethyl)quinuclidin-3-one,
(1S,2R,4S)-5,5,8,8-tetrafluoro-2-(hydroxymethyl)-2-(methoxymethyl)-4-methylquinuclidin-3-one,
(1R,2S,4R)-5,5,8,8-tetrafluoro-2-(hydroxymethyl)-2-(methoxymethyl)-4-methylquinuclidin-3-one,
(1R,2R,4S,5R,8S)-2-(hydroxymethyl)-2-(methoxymethyl)-4,5,8-trimethylquinuclidin-3-one,
(1R,2S,4R)-2-(ethoxymethyl)-2-(hydroxymethyl)-4-methylquinuclidin-3-one,
(1S,2R,4S)-2-(ethoxymethyl)-2-(hydroxymethyl)-4-methylquinuclidin-3-one,
(1R,2S,4R)-2-(hydroxymethyl)-2-(isopropoxymethyl)-4-methylquinuclidin-3-one,
(1S,2R,4S)-2-(hydroxymethyl)-2-(isopropoxymethyl)-4-methylquinuclidin-3-one,
(1R,2S,4R)-2-(tert-butoxymethyl)-2-(hydroxymethyl)-4-methylquinuclidin-3-one,
(1S,2R,4S)-2-(tert-butoxymethyl)-2-(hydroxymethyl)-4-methylquinuclidin-3-one
(1R,2S,4R)-2-(hydroxymethyl)-2-(isobutoxymethyl)-4-methylquinuclidin-3-one,
(1S,2R,4S)-2-(hydroxymethyl)-2-(isobutoxymethyl)-4-methylquinuclidin-3-one
(1R,2S,4R)-2-(cyclopropoxymethyl)-2-(hydroxymethyl)-4-methylquinuclidin-3-one,
(1S,2R,4S)-2-(cyclopropoxymethyl)-2-(hydroxymethyl)-4-methylquinuclidin-3-one,
(1S,2S,4S)-2-(hydroxymethyl)-2-(methoxymethyl)-4-methyl-3-oxoquinuclidine 1-oxide,
(1S,2R,4S)-2-(hydroxymethyl)-4-methyl-2-((methylamino)methyl)quinuclidin-3-one,
2-((1S,2R,4S)-2-(hydroxymethyl)-4-methyl-3-oxoquinuclidin-2-yl)acetonitrile,
(1S,2R,4S)-2-(but-2-yn-1-yl)-2-(hydroxymethyl)-4-methylquinuclidin-3-one,
(1S,2R,4S)-2-allyl-2-(hydroxymethyl)-4-methylquinuclidin-3-one,
(1S,2R,4S)-2-(hydroxymethyl)-4-methyl-2-((methylthio)methyl)quinuclidin-3-one,
(1R,2S,4R)-2-(hydroxymethyl)-4-methyl-2-((methylthio)methyl)quinuclidin-3-one,
(1R,2S,4R)-2-(mercaptomethyl)-4-methyl-2-((methylthio)methyl)quinuclidin-3-one,
(1S,2R,4S)-2-(mercaptomethyl)-4-methyl-2-((methylthio)methyl)quinuclidin-3-one,
(1R,2S,4R)-2-(ethoxymethyl)-5,5-difluoro-2-(hydroxymethyl)-4-methylquinuclidin-3-one,
(1R,2S,4R)-2-(cyclopropoxymethyl)-5,5-difluoro-2-(hydroxymethyl)-4-methylquinuclidin-3-one,
(1R,2S,4R)-5,5-difluoro-2-(hydroxymethyl)-2-(isopropoxymethyl)-4-methylquinuclidin-3-one,
((1R,2R,4R)-2-(ethoxymethyl)-5,5-difluoro-4-methyl-3-oxoquinuclidin-2-yl)methyl acetate,
((1R,2R,4R)-2-(ethoxymethyl)-5,5-difluoro-4-methyl-3-oxoquinuclidin-2-yl)methyl isobutyrate,
((1R,2R,4R)-2-(ethoxymethyl)-5,5-difluoro-4-methyl-3-oxoquinuclidin-2-yl)methyl pivalate,
isopropyl ((((1R,2R,4R)-5,5-difluoro-2-(methoxymethyl)-4-methyl-3-oxoquinuclidin-2-yl)methoxy)(phenoxy)phosphoryl)-L-alaninate,
isopropyl (((2-(methoxymethyl)-3-oxoquinuclidin-2-yl)methoxy)(phenoxy)phosphoryl)-L-alaninate,
isopropyl (((3-oxoquinuclidin-2-yl)methoxy)(phenoxy)phosphoryl)-L-alaninate,
neopentyl (((3-oxoquinuclidin-2-yl)methoxy)(phenoxy)phosphoryl)-L-alaninate,
benzyl (((3-oxoquinuclidin-2-yl)methoxy)(phenoxy)phosphoryl)-L-alaninate,
2-ethylbutyl (((3-oxoquinuclidin-2-yl)methoxy)(phenoxy)phosphoryl)-L-alaninate,
isopropyl ((((R)-3-oxoquinuclidin-2-yl)methoxy)(phenoxy)phosphoryl)-L-alaninate,
neopentyl ((((R)-3-oxoquinuclidin-2-yl)methoxy)(phenoxy)phosphoryl)-L-alaninate,
benzyl ((((R)-3-oxoquinuclidin-2-yl)methoxy)(phenoxy)phosphoryl)-L-alaninate,
2-ethylbutyl ((((R)-3-oxoquinuclidin-2-yl)methoxy)(phenoxy)phosphoryl)-L-alaninate,
isopropyl ((((S)-3-oxoquinuclidin-2-yl)methoxy)(phenoxy)phosphoryl)-L-alaninate,
neopentyl ((((S)-3-oxoquinuclidin-2-yl)methoxy)(phenoxy)phosphoryl)-L-alaninate,
benzyl ((((S)-3-oxoquinuclidin-2-yl)methoxy)(phenoxy)phosphoryl)-L-alaninate,
2-ethylbutyl ((((S)-3-oxoquinuclidin-2-yl)methoxy)(phenoxy)phosphoryl)-L-alaninate,
isopropyl (((3-oxo-1-azabicyclo[2.2.1]heptan-2-yl)methoxy)(phenoxy)phosphoryl)-L-alaninate,
neopentyl (((3-oxo-1-azabicyclo[2.2.1]heptan-2-yl)methoxy)(phenoxy)phosphoryl)-L-alaninate,
benzyl (((3-oxo-1-azabicyclo[2.2.1]heptan-2-yl)methoxy)(phenoxy)phosphoryl)-L-alaninate,
2-ethylbutyl (((3-oxo-1-azabicyclo[2.2.1]heptan-2-yl)methoxy)(phenoxy)phosphoryl)-L-alaninate,
isopropyl ((((1S,4R)-3-oxo-1-azabicyclo[2.2.1]heptan-2-yl)methoxy)(phenoxy)phosphoryl)-L-alaninate,
neopentyl ((((1S,4R)-3-oxo-1-azabicyclo[2.2.1]heptan-2-yl)methoxy)(phenoxy)phosphoryl)-L-alaninate,
benzyl ((((1S,4R)-3-oxo-1-azabicyclo[2.2.1]heptan-2-yl)methoxy)(phenoxy)phosphoryl)-L-alaninate,
2-ethylbutyl ((((1S,4R)-3-oxo-1-azabicyclo[2.2.1]heptan-2-yl)methoxy)(phenoxy)phosphoryl)-L-alaninate,
isopropyl ((((1R,4S)-3-oxo-1-azabicyclo[2.2.1]heptan-2-yl)methoxy)(phenoxy)phosphoryl)-L-alaninate,
neopentyl ((((1R,4S)-3-oxo-1-azabicyclo[2.2.1]heptan-2-yl)methoxy)(phenoxy)phosphoryl)-L-alaninate,
benzyl ((((1R,4S)-3-oxo-1-azabicyclo[2.2.1]heptan-2-yl)methoxy)(phenoxy)phosphoryl)-L-alaninate,
2-ethylbutyl ((((1R,4S)-3-oxo-1-azabicyclo[2.2.1]heptan-2-yl)methoxy)(phenoxy)phosphoryl)-L-alaninate,
isopropyl ((((2R)-3-oxo-1-azabicyclo[2.2.1]heptan-2-yl)methoxy)(phenoxy)phosphoryl)-L-alaninate,
neopentyl ((((2R)-3-oxo-1-azabicyclo[2.2.1]heptan-2-yl)methoxy)(phenoxy)phosphoryl)-L-alaninate,
benzyl ((((2R)-3-oxo-1-azabicyclo[2.2.1]heptan-2-yl)methoxy)(phenoxy)phosphoryl)-L-alaninate,
2-ethylbutyl ((((2R)-3-oxo-1-azabicyclo[2.2.1]heptan-2-yl)methoxy)(phenoxy)phosphoryl)-L-alaninate,
isopropyl ((((1S,2R,4R)-3-oxo-1-azabicyclo[2.2.1]heptan-2-yl)methoxy)(phenoxy)phosphoryl)-L-alaninate,
neopentyl ((((1S,2R,4R)-3-oxo-1-azabicyclo[2.2.1]heptan-2-yl)methoxy)(phenoxy)phosphoryl)-L-alaninate,
benzyl ((((1S,2R,4R)-3-oxo-1-azabicyclo[2.2.1]heptan-2-yl)methoxy)(phenoxy)phosphoryl)-L-alaninate,
2-ethylbutyl ((((1S,2R,4R)-3-oxo-1-azabicyclo[2.2.1]heptan-2-yl)methoxy)(phenoxy)phosphoryl)-L-alaninate,
isopropyl ((((1R,2R,4S)-3-oxo-1-azabicyclo[2.2.1]heptan-2-yl)methoxy)(phenoxy)phosphoryl)-L-alaninate,
neopentyl ((((1R,2R,4S)-3-oxo-1-azabicyclo[2.2.1]heptan-2-yl)methoxy)(phenoxy)phosphoryl)-L-alaninate,
benzyl ((((1R,2R,4S)-3-oxo-1-azabicyclo[2.2.1]heptan-2-yl)methoxy)(phenoxy)phosphoryl)-L-alaninate,
2-ethylbutyl ((((1R,2R,4S)-3-oxo-1-azabicyclo[2.2.1]heptan-2-yl)methoxy)(phenoxy)phosphoryl)-L-alaninate,
isopropyl ((((2S)-3-oxo-1-azabicyclo[2.2.1]heptan-2-yl)methoxy)(phenoxy)phosphoryl)-L-alaninate,
neopentyl ((((2S)-3-oxo-1-azabicyclo[2.2.1]heptan-2-yl)methoxy)(phenoxy)phosphoryl)-L-alaninate,
benzyl ((((2S)-3-oxo-1-azabicyclo[2.2.1]heptan-2-yl)methoxy)(phenoxy)phosphoryl)-L-alaninate,
2-ethylbutyl ((((2S)-3-oxo-1-azabicyclo[2.2.1]heptan-2-yl)methoxy)(phenoxy)phosphoryl)-L-alaninate,
isopropyl ((((1S,2S,4R)-3-oxo-1-azabicyclo[2.2.1]heptan-2-yl)methoxy)(phenoxy)phosphoryl)-L-alaninate,
neopentyl ((((1S,2S,4R)-3-oxo-1-azabicyclo[2.2.1]heptan-2-yl)methoxy)(phenoxy)phosphoryl)-L-alaninate,
benzyl ((((1S,2S,4R)-3-oxo-1-azabicyclo[2.2.1]heptan-2-yl)methoxy)(phenoxy)phosphoryl)-L-alaninate,
2-ethylbutyl ((((1S,2S,4R)-3-oxo-1-azabicyclo[2.2.1]heptan-2-yl)methoxy)(phenoxy)phosphoryl)-L-alaninate,
isopropyl ((((1R,2S,4S)-3-oxo-1-azabicyclo[2.2.1]heptan-2-yl)methoxy)(phenoxy)phosphoryl)-L-alaninate,
neopentyl ((((1R,2S,4S)-3-oxo-1-azabicyclo[2.2.1]heptan-2-yl)methoxy)(phenoxy)phosphoryl)-L-alaninate,
benzyl ((((1R,2S,4S)-3-oxo-1-azabicyclo[2.2.1]heptan-2-yl)methoxy)(phenoxy)phosphoryl)-L-alaninate,
2-ethylbutyl ((((1R,2S,4S)-3-oxo-1-azabicyclo[2.2.1]heptan-2-yl)methoxy)(phenoxy)phosphoryl)-L-alaninate,
(1R,2S,4R)-5,5-difluoro-2-(hydroxymethyl)-2-(methoxymethyl)quinuclidin-3-one,
(1S,2S,4S)-5,5-difluoro-2-(hydroxymethyl)-2-(methoxymethyl)quinuclidin-3-one,
(1R,2R,4R)-5,5-difluoro-2-(hydroxymethyl)-2-(methoxymethyl)quinuclidin-3-one,
(1S,2R,4S)-5,5-difluoro-2-(hydroxymethyl)-2-(methoxymethyl)quinuclidin-3-one.
(1R,2S,4S)-2-(hydroxymethyl)-2-(methoxymethyl)-5,5-dimethylquinuclidin-3-one,
(1S,2S,4R)-2-(hydroxymethyl)-2-(methoxymethyl)-5,5-dimethylquinuclidin-3-one,
(1R,2R,4S)-2-(hydroxymethyl)-2-(methoxymethyl)-5,5-dimethylquinuclidin-3-one,
(1S,2R,4R)-2-(hydroxymethyl)-2-(methoxymethyl)-5,5-dimethylquinuclidin-3-one,
(1R,2S,4S,5S)-2-(hydroxymethyl)-2-(methoxymethyl)-5-methylquinuclidin-3-one,
(1S,2S,4R,5S)-2-(hydroxymethyl)-2-(methoxymethyl)-5-methylquinuclidin-3-one,
(1R,2R,4S,5S)-2-(hydroxymethyl)-2-(methoxymethyl)-5-methylquinuclidin-3-one,
(1S,2R,4R,5S)-2-(hydroxymethyl)-2-(methoxymethyl)-5-methylquinuclidin-3-one,
(1R,2S,4S,5R)-2-(hydroxymethyl)-2-(methoxymethyl)-5-methylquinuclidin-3-one,
(1S,2S,4R,5R)-2-(hydroxymethyl)-2-(methoxymethyl)-5-methylquinuclidin-3-one,
(1R,2R,4S,5R)-2-(hydroxymethyl)-2-(methoxymethyl)-5-methylquinuclidin-3-one,
(1S,2R,4R,5R)-2-(hydroxymethyl)-2-(methoxymethyl)-5-methylquinuclidin-3-one,
(1R,2S,4S,5S)-2-(hydroxymethyl)-2-(methoxymethyl)-5-(trifluoromethyl)quinuclidin-3-one,
(1S,2S,4R,5S)-2-(hydroxymethyl)-2-(methoxymethyl)-5-(trifluoromethyl)quinuclidin-3-one,
(1R,2R,4S,5S)-2-(hydroxymethyl)-2-(methoxymethyl)-5-(trifluoromethyl)quinuclidin-3-one,
(1S,2R,4R,5S)-2-(hydroxymethyl)-2-(methoxymethyl)-5-(trifluoromethyl)quinuclidin-3-one,
(1R,2S,4S,5R)-2-(hydroxymethyl)-2-(methoxymethyl)-5-(trifluoromethyl)quinuclidin-3-one,
(1S,2S,4R,5R)-2-(hydroxymethyl)-2-(methoxymethyl)-5-(trifluoromethyl)quinuclidin-3-one,
(1R,2R,4S,5R)-2-(hydroxymethyl)-2-(methoxymethyl)-5-(trifluoromethyl)quinuclidin-3-one,
(1S,2R,4R,5R)-2-(hydroxymethyl)-2-(methoxymethyl)-5-(trifluoromethyl)quinuclidin-3-one,
(1S,4R,5S)-5-(hydroxymethyl)-5-(methoxymethyl)-4-azaspiro[bicyclo[2.2.2]octane-2,1′-cyclopropan]-6-one,
(1R,3 S,4S,6S)-6-(hydroxymethyl)-6-(methoxymethyl)-5-oxoquinuclidine-3-carbonitrile,
(1R,2S,4S)-2-(hydroxymethyl)-5-methoxy-2-(methoxymethyl)-5-methylquinuclidin-3-one,
(1R,2S,4S)-5-hydroxy-2-(hydroxymethyl)-2-(methoxymethyl)-5-methylquinuclidin-3-one,
(1R,2S,4S,5R)-2-(hydroxymethyl)-2-(methoxymethyl)-5-phenylquinuclidin-3-one,
(1R,2S,4S,5S)-2-(hydroxymethyl)-2-(methoxymethyl)-5-(pyridin-2-yl)quinuclidin-3-one,
(1R,2S,4S,5R)-2-(hydroxymethyl)-2-(methoxymethyl)-5-(pyridin-3-yl)quinuclidin-3-one,
(1R,2S,4S,5R)-2-(hydroxymethyl)-2-(methoxymethyl)-5-(pyridin-4-yl)quinuclidin-3-one,
((1R,2R,4R)-5,5-difluoro-2-(methoxymethyl)-3-oxoquinuclidin-2-yl)methyl isobutyrate,
((1R,2R,4R)-5,5-difluoro-2-(isopropoxymethyl)-3-oxoquinuclidin-2-yl)methyl isobutyrate,
isopropyl ((R)-(((1R,2R,4R)-5,5-difluoro-2-(methoxymethyl)-3-oxoquinuclidin-2-yl)methoxy)(phenoxy)phosphoryl)-L-alaninate,
bis(((1S,2S,4S)-2-(methoxymethyl)-3-oxoquinuclidin-2-yl)methyl) carbonate,
(1S,1'S,2S,2'S,4S,4'S)-2,2′-(oxybis(methylene))bis(2-(methoxymethyl)quinuclidin-3-one),
bis(((1S,2S,4S)-2-(methoxymethyl)-3-oxoquinuclidin-2-yl)methyl) piperazine-1,4-dicarboxylate,
bis(((1S,2S,4S)-2-(methoxymethyl)-3-oxoquinuclidin-2-yl)methyl) ((1R,3S)-cyclohexane-1,3-diyl)dicarbamate,
bis(((1S,2R,4S)-2-(methoxymethyl)-3-oxoquinuclidin-2-yl)methyl) fumarate bis(((1S,2R,4S)-2-(methoxymethyl)-3-oxoquinuclidin-2-yl)methyl) terephthalate,
bis(((1S,2R,4S)-2-(methoxymethyl)-3-oxoquinuclidin-2-yl)methyl) phosphoramidate,
benzyl (bis(((1S,2R,4S)-2-(methoxymethyl)-3-oxoquinuclidin-2-yl)methoxy)phosphoryl)-L-alaninate,
isopropyl (bis(((1S,2R,4S)-2-(methoxymethyl)-3-oxoquinuclidin-2-yl)methoxy)phosphoryl)-L-phenylalaninate,
bis(((1 S,2R,4S)-2-(methoxymethyl)-3-oxoquinuclidin-2-yl)methyl) phenylphosphoramidate,
bis(((1 S,2R,4S)-2-(methoxymethyl)-3-oxoquinuclidin-2-yl)methyl) benzylphosphoramidate,
isopropyl (bis(((1S,2R,4S)-2-(methoxymethyl)-3-oxoquinuclidin-2-yl)methoxy)phosphoryl)-L-alaninate,
isopropyl (bis(((1S,2R,4S)-2-(methoxymethyl)-3-oxoquinuclidin-2-yl)methoxy)phosphoryl)-L-valinate,
bis(((1S,2R,4S)-2-(methoxymethyl)-3-oxoquinuclidin-2-yl)methyl) hydrogen phosphate,
bis(((1S,2R,4S)-2-(methoxymethyl)-3-oxoquinuclidin-2-yl)methyl) phenyl phosphate,
bis(((1S,2R,4S)-2-(methoxymethyl)-3-oxoquinuclidin-2-yl)methyl) naphthalen-1-yl phosphate,
bis(((1S,2R,4S)-2-(methoxymethyl)-3-oxoquinuclidin-2-yl)methyl) p-tolyl phosphate benzyl bis(((1S,2R,4S)-2-(methoxymethyl)-3-oxoquinuclidin-2-yl)methyl) phosphate,
isopropyl bis(((1S,2R,4S)-2-(methoxymethyl)-3-oxoquinuclidin-2-yl)methyl) phosphate,
((bis(((1S,2R,4S)-2-(methoxymethyl)-3-oxoquinuclidin-2-yl)methoxy)phosphoryl)oxy)methyl pivalate,
tris((2-(methoxymethyl)-3-oxoquinuclidin-2-yl)methyl) phosphate,
bis(((1 S,2R,4S)-2-(methoxymethyl)-3-oxoquinuclidin-2-yl)methyl)
methylphosphonate,
bis(((1 S,2R,4S)-2-(methoxymethyl)-3-oxoquinuclidin-2-yl)methyl) isopropylphosphonate,
bis(((1 S,2R,4S)-2-(methoxymethyl)-3-oxoquinuclidin-2-yl)methyl) phenylphosphonate,
((1S,2R,4S)-2-(methoxymethyl)-3-oxoquinuclidin-2-yl)methyl ((((1S,2R,4S)-2-(methoxymethyl)-3-oxoquinuclidin-2-yl)methoxy)carbonyl)-L-valinate,
((1S,2R,4S)-2-(methoxymethyl)-3-oxoquinuclidin-2-yl)methyl ((((1S,2R,4S)-2-(methoxymethyl)-3-oxoquinuclidin-2-yl)methoxy)carbonyl)-L-phenylalaninate,
bis(((1S,2R,4S)-2-(methoxymethyl)-3-oxoquinuclidin-2-yl)methyl) (S)-pyrrolidine-1,2-dicarboxylate,
((1S,2R,4S)-2-(methoxymethyl)-3-oxoquinuclidin-2-yl)methyl ((((1S,2R,4S)-2-(methoxymethyl)-3-oxoquinuclidin-2-yl)methoxy)carbonyl)glycinate,
(1S,1′S,2S,2′S,4S,4′S)-2,2′-((methylenebis(oxy))bis(methylene))bis(2-(methoxymethyl)quinuclidin-3-one),
(1S,1′S,2S,2′S,4S,4′S)-2,2′-((ethane-1,2-diylbis(oxy))bis(methylene))bis(2-(methoxymethyl)quinuclidin-3-one),
(1R,2R,4R)-2-(aminomethyl)-2-(methoxymethyl)quinuclidin-3-one,
((1S,2S,4S)-2-(methoxymethyl)-3-oxoquinuclidin-2-yl)methyl (((1R,2R,4R)-2-(methoxymethyl)-3-oxoquinuclidin-2-yl)methyl)(methyl)carbamate,
((1S,2S,4S)-2-(methoxymethyl)-3-oxoquinuclidin-2-yl)methyl (((1R,2R,4R)-2-(methoxymethyl)-3-oxoquinuclidin-2-yl)methyl)carbamate,
((1S,2S,4S)-2-(methoxymethyl)-3-oxoquinuclidin-2-yl)methyl ethyl(((1R,2R,4R)-2-(methoxymethyl)-3-oxoquinuclidin-2-yl)methyl)carbamate,
bis(((1S,2R,4S)-2-(methoxymethyl)-3-oxoquinuclidin-2-yl)methyl) ethane-1,2-diylbis(methylcarbamate),
((1S,2R,4S)-2-(methoxymethyl)-3-oxoquinuclidin-2-yl)methyl (4-((((1S,2R,4S)-2-(methoxymethyl)-3-oxoquinuclidin-2-yl)methoxy)methyl)phenyl)carbamate,
bis(((1S,2S,4S)-2-(methoxymethyl)-3-oxoquinuclidin-2-yl)methyl) (3aS,6aS)-tetrahydropyrrolo[3,4-c]pyrrole-2,5(1H,3H)-dicarboxylate,
bis(((1S,2S,4S)-2-(methoxymethyl)-3-oxoquinuclidin-2-yl)methyl) 3,9-diazaspiro[5.5]undecane-3,9-dicarboxylate,
bis(((1S,2S,4S)-2-(methoxymethyl)-3-oxoquinuclidin-2-yl)methyl) 2,7-diazaspiro[3.5]nonane-2,7-dicarboxylate,
bis(((1S,2S,4S)-2-(methoxymethyl)-3-oxoquinuclidin-2-yl)methyl) ((1R,2S)-cyclohexane-1,2-diyl)dicarbamate,
bis(((1S,2S,4S)-2-(methoxymethyl)-3-oxoquinuclidin-2-yl)methyl) ((1S,4S)-cyclohexane-1,4-diyl)dicarbamate,
bis(((1S,2S,4S)-2-(methoxymethyl)-3-oxoquinuclidin-2-yl)methyl) 1,4-diazepane-1,4-dicarboxylate,
bis(((1S,2S,4S)-2-(methoxymethyl)-3-oxoquinuclidin-2-yl)methyl) sulfate,
bis(((1S,2S,4S)-2-(methoxymethyl)-3-oxoquinuclidin-2-yl)methyl) sulfurimidate,
bis(((1S,2R,4S)-2-(methoxymethyl)-3-oxoquinuclidin-2-yl)methyl) pyridine-2,6-dicarboxylate,
bis(((1S,2R,4S)-2-(methoxymethyl)-3-oxoquinuclidin-2-yl)methyl) pyridine-2,5-dicarboxylate,
bis(((1S,2R,4S)-2-(methoxymethyl)-3-oxoquinuclidin-2-yl)methyl) cyclohexane-1,4-dicarboxylate,
bis(((1S,2R,4S)-2-(methoxymethyl)-3-oxoquinuclidin-2-yl)methyl) malonate,
bis(((1S,2R,4S)-2-(methoxymethyl)-3-oxoquinuclidin-2-yl)methyl) succinate,
bis(((1S,2R,4S)-2-(methoxymethyl)-3-oxoquinuclidin-2-yl)methyl) succinate,
(1S,1′S,2R,2′R,4S,4′S)-2,2′-((methylenebis(oxy))bis(methylene))bis(2-(methoxymethyl)quinuclidin-3-one),
(1S,1′S,2S,2′S,4S,4′S)-2,2′-((ethane-1,2-diylbis(oxy))bis(methylene))bis(2-(methoxymethyl)quinuclidin-3-one),
(1S,1′S,2S,2′S,4S,4′S)-2,2′-((ethane-1,2-diylbis(oxy))bis(methylene))bis(2-(methoxymethyl)quinuclidin-3-one),

List 2
(1R,2S,4S)-2-(hydroxymethyl)-2-(methoxymethyl)-6,6-dimethylquinuclidin-3-one,
(1S,2S,4R)-2-(hydroxymethyl)-2-(methoxymethyl)-6,6-dimethylquinuclidin-3-one,
(1R,2R,4S)-2-(hydroxymethyl)-2-(methoxymethyl)-6,6-dimethylquinuclidin-3-one,
(1S,2R,4R)-2-(hydroxymethyl)-2-(methoxymethyl)-6,6-dimethylquinuclidin-3-one,
(1R,2S,4S,6S)-2-(hydroxymethyl)-2-(methoxymethyl)-6-methylquinuclidin-3-one,
(1S,2S,4R,6S)-2-(hydroxymethyl)-2-(methoxymethyl)-6-methylquinuclidin-3-one,
(1R,2R,4S,6S)-2-(hydroxymethyl)-2-(methoxymethyl)-6-methylquinuclidin-3-one,
(1S,2R,4R,6S)-2-(hydroxymethyl)-2-(methoxymethyl)-6-methylquinuclidin-3-one,
(1R,2S,4S,6R)-2-(hydroxymethyl)-2-(methoxymethyl)-6-methylquinuclidin-3-one,
(1S,2S,4R,6R)-2-(hydroxymethyl)-2-(methoxymethyl)-6-methylquinuclidin-3-one,
(1R,2R,4S,6R)-2-(hydroxymethyl)-2-(methoxymethyl)-6-methylquinuclidin-3-one,
(1S,2R,4R,6R)-2-(hydroxymethyl)-2-(methoxymethyl)-6-methylquinuclidin-3-one,
(1R,2S,4S,6R)-2-(hydroxymethyl)-2-(methoxymethyl)-6-(trifluoromethyl)quinuclidin-3-one,
(1S,2S,4R,6R)-2-(hydroxymethyl)-2-(methoxymethyl)-6-(trifluoromethyl)quinuclidin-3-one,
(1R,2R,4S,6R)-2-(hydroxymethyl)-2-(methoxymethyl)-6-(trifluoromethyl)quinuclidin-3-one,
(1S,2R,4R,6R)-2-(hydroxymethyl)-2-(methoxymethyl)-6-(trifluoromethyl)quinuclidin-3-one,
(1R,2S,4S,6S)-2-(hydroxymethyl)-2-(methoxymethyl)-6-(trifluoromethyl)quinuclidin-3-one,
(1S,2S,4R,6S)-2-(hydroxymethyl)-2-(methoxymethyl)-6-(trifluoromethyl)quinuclidin-3-one,
(1R,2R,4S,6S)-2-(hydroxymethyl)-2-(methoxymethyl)-6-(trifluoromethyl)quinuclidin-3-one,
(1S,2R,4R,6S)-2-(hydroxymethyl)-2-(methoxymethyl)-6-(trifluoromethyl)quinuclidin-3-one,
(1R,2S,4S,6S)-6-ethyl-2-(hydroxymethyl)-2-(methoxymethyl)quinuclidin-3-one,
(1S,2S,4R,6S)-6-ethyl-2-(hydroxymethyl)-2-(methoxymethyl)quinuclidin-3-one,
(1R,2R,4S,6S)-6-ethyl-2-(hydroxymethyl)-2-(methoxymethyl)quinuclidin-3-one,
(1S,2R,4R,6S)-6-ethyl-2-(hydroxymethyl)-2-(methoxymethyl)quinuclidin-3-one,
(1R,2S,4S,6R)-6-ethyl-2-(hydroxymethyl)-2-(methoxymethyl)quinuclidin-3-one,
(1S,2S,4R,6R)-6-ethyl-2-(hydroxymethyl)-2-(methoxymethyl)quinuclidin-3-one,
(1R,2R,4S,6R)-6-ethyl-2-(hydroxymethyl)-2-(methoxymethyl)quinuclidin-3-one,
(1S,2R,4R,6R)-6-ethyl-2-(hydroxymethyl)-2-(methoxymethyl)quinuclidin-3-one,
(1R,2S,4S,6R)-2-(hydroxymethyl)-6-isopropyl-2-(methoxymethyl)quinuclidin-3-one,
(1S,2S,4R,6R)-2-(hydroxymethyl)-6-isopropyl-2-(methoxymethyl)quinuclidin-3-one,
(1R,2R,4S,6R)-2-(hydroxymethyl)-6-isopropyl-2-(methoxymethyl)quinuclidin-3-one,
(1S,2R,4R,6R)-2-(hydroxymethyl)-6-isopropyl-2-(methoxymethyl)quinuclidin-3-one,
(1R,2S,4S,6S)-2-(hydroxymethyl)-6-isopropyl-2-(methoxymethyl)quinuclidin-3-one,
(1S,2S,4R,6S)-2-(hydroxymethyl)-6-isopropyl-2-(methoxymethyl)quinuclidin-3-one,
(1R,2R,4S,6S)-2-(hydroxymethyl)-6-isopropyl-2-(methoxymethyl)quinuclidin-3-one,
(1S,2R,4R,6S)-2-(hydroxymethyl)-6-isopropyl-2-(methoxymethyl)quinuclidin-3-one,
(1R,2S,4S,6R)-6-(tert-butyl)-2-(hydroxymethyl)-2-(methoxymethyl)quinuclidin-3-one,
(1S,2S,4R,6R)-6-(tert-butyl)-2-(hydroxymethyl)-2-(methoxymethyl)quinuclidin-3-one,
(1R,2R,4S,6R)-6-(tert-butyl)-2-(hydroxymethyl)-2-(methoxymethyl)quinuclidin-3-one,
(1S,2R,4R,6R)-6-(tert-butyl)-2-(hydroxymethyl)-2-(methoxymethyl)quinuclidin-3-one,
(1R,2S,4S,6S)-6-(tert-butyl)-2-(hydroxymethyl)-2-(methoxymethyl)quinuclidin-3-one,
(1S,2S,4R,6S)-6-(tert-butyl)-2-(hydroxymethyl)-2-(methoxymethyl)quinuclidin-3-one,
(1R,2R,4S,6S)-6-(tert-butyl)-2-(hydroxymethyl)-2-(methoxymethyl)quinuclidin-3-one
(1S,2R,4R,6S)-6-(tert-butyl)-2-(hydroxymethyl)-2-(methoxymethyl)quinuclidin-3-one,
(1R,2R,4S,6S)-2-(hydroxymethyl)-2-(methoxymethyl)-6-(1,1,1-trifluoro-2-methylpropan-2-yl)quinuclidin-3-one,
(1R,2R,4S,6S)-6-cyclohexyl-2-(hydroxymethyl)-2-(methoxymethyl)quinuclidin-3-one,
(1R,2R,4S,6S)-6-cyclobutyl-2-(hydroxymethyl)-2-(methoxymethyl)quinuclidin-3-one,
(1R,2R,4S,6S)-6-cyclopropyl-2-(hydroxymethyl)-2-(methoxymethyl)quinuclidin-3-one,
(1R,2R,4S,6S)-6-cyclopentyl-2-(hydroxymethyl)-2-(methoxymethyl)quinuclidin-3-one,
(1R,2R,4S,6S)-2-(hydroxymethyl)-6-(2-hydroxypropan-2-yl)-2-(methoxymethyl)quinuclidin-3-one,
(1R,2R,4S,6S)-6-(2-aminopropan-2-yl)-2-(hydroxymethyl)-2-(methoxymethyl)quinuclidin-3-one,
2-((1R,2S,4S,6R)-6-(hydroxymethyl)-6-(methoxymethyl)-5-oxoquinuclidin-2-yl)-2-methylpropanenitrile,
(6R)-2-(hydroxymethyl)-2-(methoxymethyl)-6-phenylquinuclidin-3-one,
(6R)-2-(hydroxymethyl)-2-(methoxymethyl)-6-phenylquinuclidin-3-one,
(6S)-2-(hydroxymethyl)-2-(methoxymethyl)-6-phenylquinuclidin-3-one,
(6S)-2-(hydroxymethyl)-2-(methoxymethyl)-6-phenylquinuclidin-3-one,
(6R)-2-(hydroxymethyl)-2-(methoxymethyl)-6-(4-(trifluoromethyl)phenyl)quinuclidin-3-one,
(6R)-2-(hydroxymethyl)-2-(methoxymethyl)-6-(4-(trifluoromethyl)phenyl)quinuclidin-3-one,
(6S)-2-(hydroxymethyl)-2-(methoxymethyl)-6-(4-(trifluoromethyl)phenyl)quinuclidin-3-one,
(6S)-2-(hydroxymethyl)-2-(methoxymethyl)-6-(4-(trifluoromethyl)phenyl)quinuclidin-3-one,
(6S)-2-(hydroxymethyl)-2-(methoxymethyl)-6-(p-tolyl)quinuclidin-3-one,
(6S)-6-(4-chloro-2-fluorophenyl)-2-(hydroxymethyl)-2-(methoxymethyl)quinuclidin-3-one,
(6S)-6-(2-chloro-4-isopropylphenyl)-2-(hydroxymethyl)-2-(methoxymethyl)quinuclidin-3-one,
(6S)-2-(hydroxymethyl)-6-(4-isopropyl-2-methoxyphenyl)-2-(methoxymethyl)quinuclidin-3-one,
(6S)-2-(hydroxymethyl)-6-(4-(isopropylsulfonyl)phenyl)-2-(methoxymethyl)quinuclidin-3-one,
(6R)-2-(hydroxymethyl)-2-(methoxymethyl)-6-(pyridin-4-yl)quinuclidin-3-one,
(6R)-2-(hydroxymethyl)-2-(methoxymethyl)-6-(pyridin-4-yl)quinuclidin-3-one,
(6S)-2-(hydroxymethyl)-2-(methoxymethyl)-6-(pyridin-4-yl)quinuclidin-3-one,
(6S)-2-(hydroxymethyl)-2-(methoxymethyl)-6-(pyridin-4-yl)quinuclidin-3-one,
(6S)-2-(hydroxymethyl)-2-(methoxymethyl)-6-(pyridin-2-yl)quinuclidin-3-one,
(6S)-2-(hydroxymethyl)-2-(methoxymethyl)-6-(pyridin-3-yl)quinuclidin-3-one,
(6S)-2-(hydroxymethyl)-2-(methoxymethyl)-6-(pyridin-4-yl)quinuclidin-3-one
(1S,2S,4R,6R,7S)-2-(hydroxymethyl)-2-(methoxymethyl)-6,7-dimethylquinuclidin-3-one,
(1S,2S,4R,6R,7S)-2-(hydroxymethyl)-2-(methoxymethyl)-6,7-dimethylquinuclidin-3-one,
(1R,2S,4S,6R,7S)-2-(hydroxymethyl)-6,7-diisopropyl-2-(methoxymethyl)quinuclidin-3-one,
(1R,2S,4S,6R,7S)-2-(hydroxymethyl)-6,7-diisopropyl-2-(methoxymethyl)quinuclidin-3-one,
(1R,2S,4S,6R,7S)-2-(hydroxymethyl)-6-isopropyl-2-(methoxymethyl)-7-(trifluoromethyl)quinuclidin-3-one,
(1R,2S,4S,6R,7S)-2-(hydroxymethyl)-6-isopropyl-2-(methoxymethyl)-7-(trifluoromethyl)quinuclidin-3-one,
(1S,2S,4R,6S,7R)-2-(hydroxymethyl)-6-isopropyl-2-(methoxymethyl)-7-(trifluoromethyl)quinuclidin-3-one,
(1S,2S,4R,6S,7R)-2-(hydroxymethyl)-6-isopropyl-2-(methoxymethyl)-7-(trifluoromethyl)quinuclidin-3-one,
((1S,2R,4R,6S)-2-(methoxymethyl)-6-methyl-3-oxoquinuclidin-2-yl)methyl acetate,
((1S,2R,4R,6S)-2-(methoxymethyl)-6-methyl-3-oxoquinuclidin-2-yl)methyl isobutyrate,
((1S,2R,4R,6S)-2-(methoxymethyl)-6-methyl-3-oxoquinuclidin-2-yl)methyl pivalate,
((((((1S,2R,4R,6S)-2-(methoxymethyl)-6-methyl-3-oxoquinuclidin-2-yl)methoxy)methyl)phosphoryl)bis(oxy))bis(methylene) bis(2,2-dimethylpropanoate),
diisopropyl (((((((1S,2R,4R,6S)-2-(methoxymethyl)-6-methyl-3-oxoquinuclidin-2-yl)methoxy)methyl)phosphoryl)bis(oxy))bis(methylene)) bis(carbonate),
isopropyl ((((1S,2R,4R,6S)-2-(methoxymethyl)-6-methyl-3-oxoquinuclidin-2-yl)methoxy)(phenoxy)phosphoryl)-L-alaninate,
isopropyl ((((1S,2R,4R,6S)-2-(methoxymethyl)-6-methyl-3-oxoquinuclidin-2-yl)methoxy)(phenoxy)phosphoryl)-L-valinate,
benzyl ((((1S,2R,4R,6S)-2-(methoxymethyl)-6-methyl-3-oxoquinuclidin-2-yl)methoxy)(phenoxy)phosphoryl)-L-alaninate,
benzyl ((((1S,2R,4R,6S)-2-(methoxymethyl)-6-methyl-3-oxoquinuclidin-2-yl)methoxy)(phenoxy)phosphoryl)-L-valinate,
isopropyl ((((1S,2R,4R,6S)-2-(methoxymethyl)-6-methyl-3-oxoquinuclidin-2-yl)methoxy)(phenoxy)phosphoryl)-L-phenylalaninate,
isopropyl (isopropoxy(((1S,2R,4R,6S)-2-(methoxymethyl)-6-methyl-3-oxoquinuclidin-2-yl)methoxy)phosphoryl)-L-phenylalaninate,
benzyl (isopropoxy(((1S,2R,4R,6S)-2-(methoxymethyl)-6-methyl-3-oxoquinuclidin-2-yl)methoxy)phosphoryl)-L-valinate,
isopropyl ((((1S,2R,4R,6S)-2-(methoxymethyl)-6-methyl-3-oxoquinuclidin-2-yl)methoxy)((S)-2-methyl-1-(propionyloxy)propoxy)phosphoryl)-L-phenylalaninate,
bis(((1S,2R,4R,6S)-2-(methoxymethyl)-6-methyl-3-oxoquinuclidin-2-yl)methyl)
methylphosphonate,
bis(((1S,2R,4R,6S)-2-(methoxymethyl)-6-methyl-3-oxoquinuclidin-2-yl)methyl) phenyl phosphate,
benzyl (bis(((1S,2R,4R,6S)-2-(methoxymethyl)-6-methyl-3-oxoquinuclidin-2-yl)methoxy)phosphoryl)-L-alaninate,
isopropyl (bis(((1S,2R,4R,6S)-2-(methoxymethyl)-6-methyl-3-oxoquinuclidin-2-yl)methoxy)phosphoryl)-L-phenylalaninate,
tris(((1S,2R,4R,6S)-2-(methoxymethyl)-6-methyl-3-oxoquinuclidin-2-yl)methyl) phosphate,
(1R,2R,4S,6R)-2-(hydroxymethyl)-2-((methoxy-d3)methyl)-6-methylquinuclidin-3-one,
(1R,2R,4S,6R)-2-(hydroxymethyl)-6-methyl-2-((trifluoromethoxy)methyl)quinuclidin-3-one,
((1R,2S,4S,6R)-2-(methoxymethyl)-6-methyl-3-oxoquinuclidin-2-yl)methyl acetate,
((1R,2S,4S,6R)-2-(methoxymethyl)-6-methyl-3-oxoquinuclidin-2-yl)methyl isobutyrate,
((1R,2S,4S,6R)-2-(methoxymethyl)-6-methyl-3-oxoquinuclidin-2-yl)methyl pivalate,
((((((1R,2S,4S,6R)-2-(methoxymethyl)-6-methyl-3-oxoquinuclidin-2-yl)methoxy)methyl)phosphoryl)bis(oxy))bis(methylene) bis(2,2-dimethylpropanoate),
diisopropyl (((((((1R,2S,4S,6R)-2-(methoxymethyl)-6-methyl-3-oxoquinuclidin-2-yl)methoxy)methyl)phosphoryl)bis(oxy))bis(methylene)) bis(carbonate),
isopropyl ((((1R,2S,4S,6R)-2-(methoxymethyl)-6-methyl-3-oxoquinuclidin-2-yl)methoxy)(phenoxy)phosphoryl)-L-alaninate,
isopropyl ((((1R,2S,4S,6R)-2-(methoxymethyl)-6-methyl-3-oxoquinuclidin-2-yl)methoxy)(phenoxy)phosphoryl)-L-valinate,
benzyl ((((1R,2S,4S,6R)-2-(methoxymethyl)-6-methyl-3-oxoquinuclidin-2-yl)methoxy)(phenoxy)phosphoryl)-L-alaninate,
benzyl ((((1R,2S,4S,6R)-2-(methoxymethyl)-6-methyl-3-oxoquinuclidin-2-yl)methoxy)(phenoxy)phosphoryl)-L-valinate,
isopropyl ((((1R,2S,4S,6R)-2-(methoxymethyl)-6-methyl-3-oxoquinuclidin-2-yl)methoxy)(phenoxy)phosphoryl)-L-phenylalaninate,
isopropyl (isopropoxy(((1R,2S,4S,6R)-2-(methoxymethyl)-6-methyl-3-oxoquinuclidin-2-yl)methoxy)phosphoryl)-L-phenylalaninate,
benzyl (isopropoxy(((1R,2S,4S,6R)-2-(methoxymethyl)-6-methyl-3-oxoquinuclidin-2-yl)methoxy)phosphoryl)-L-valinate,
isopropyl ((((1R,2S,4S,6R)-2-(methoxymethyl)-6-methyl-3-oxoquinuclidin-2-yl)methoxy)((S)-2-methyl-1-(propionyloxy)propoxy)phosphoryl)-L-phenylalaninate,
((1R,2S,4S,6R)-2-(methoxymethyl)-6-methyl-3-oxoquinuclidin-2-yl)methyl (((2S,6R)-2-(methoxymethyl)-6-methyl-3-oxoquinuclidin-2-yl)methyl)
methylphosphonate,
((1R,2S,4S,6R)-2-(methoxymethyl)-6-methyl-3-oxoquinuclidin-2-yl)methyl (((2S,6R)-2-(methoxymethyl)-6-methyl-3-oxoquinuclidin-2-yl)methyl) phenyl phosphate,
benzyl ((((1R,2S,4S,6R)-2-(methoxymethyl)-6-methyl-3-oxoquinuclidin-2-yl)methoxy)(((2S,6R)-2-(methoxymethyl)-6-methyl-3-oxoquinuclidin-2-yl)methoxy)phosphoryl)-L-alaninate,
isopropyl ((((1R,2S,4S,6R)-2-(methoxymethyl)-6-methyl-3-oxoquinuclidin-2-yl)methoxy)(((2S,6R)-2-(methoxymethyl)-6-methyl-3-oxoquinuclidin-2-yl)methoxy)phosphoryl)-L-phenylalaninate,
((1R,2S,4S,6R)-2-(methoxymethyl)-6-methyl-3-oxoquinuclidin-2-yl)methyl (((1S,2S,4R,6R)-2-(methoxymethyl)-6-methyl-3-oxoquinuclidin-2-yl)methyl) (((2S,6R)-2-(methoxymethyl)-6-methyl-3-oxoquinuclidin-2-yl)methyl) phosphate,
(1R,2S,4R)-2-(hydroxymethyl)-2-(methoxymethyl)-1-azabicyclo[2.1.1]hexan-3-one,
(1S,2R,4S)-2-(hydroxymethyl)-2-(methoxymethyl)-1-azabicyclo[2.1.1]hexan-3-one,
(1S,2S,4R)-2-(hydroxymethyl)-2-(methoxymethyl)-1-azabicyclo[2.2.1]heptan-3-one,
(1R,2S,4S)-2-(hydroxymethyl)-2-(methoxymethyl)-1-azabicyclo[2.2.1]heptan-3-one,
(1S,2R,4R)-2-(hydroxymethyl)-2-(methoxymethyl)-1-azabicyclo[2.2.1]heptan-3-one,
(1R,2R,4S)-2-(hydroxymethyl)-2-(methoxymethyl)-1-azabicyclo[2.2.1]heptan-3-one,
(1S,5R,7S)-7-(hydroxymethyl)-7-(methoxymethyl)-1-azabicyclo[3.2.1]octan-6-one,
(1R,5S,7S)-7-(hydroxymethyl)-7-(methoxymethyl)-1-azabicyclo[3.2.1]octan-6-one,
(1S,5R,7R)-7-(hydroxymethyl)-7-(methoxymethyl)-1-azabicyclo[3.2.1]octan-6-one,
(1R,5S,7R)-7-(hydroxymethyl)-7-(methoxymethyl)-1-azabicyclo[3.2.1]octan-6-one,
(1S,5R,7S)-7-(hydroxymethyl)-7-(methoxymethyl)-1-azabicyclo[3.2.2]nonan-6-one,
(1R,5S,7S)-7-(hydroxymethyl)-7-(methoxymethyl)-1-azabicyclo[3.2.2]nonan-6-one,
(1S,5R,7R)-7-(hydroxymethyl)-7-(methoxymethyl)-1-azabicyclo[3.2.2]nonan-6-one,
(1R,5S,7R)-7-(hydroxymethyl)-7-(methoxymethyl)-1-azabicyclo[3.2.2]nonan-6-one,
(1R,5R,9S)-9-(hydroxymethyl)-9-(methoxymethyl)-1-azabicyclo[3.3.2]decan-10-one,
(1S,5S,9R)-9-(hydroxymethyl)-9-(methoxymethyl)-1-azabicyclo[3.3.2]decan-10-one,

Compounds of the invention may contain one or more asymmetrically substituted carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by any known methods, such as, for example, chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and deuterium-enriched compounds. For example:
Deuterium-enriched compounds: deuterium (D or ²H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium, and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts, and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPβCD and SBEβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl.

Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl.

The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

The term “heterocycloalkyl” refers to a nonaromatic 5-8 membered monocyclic, 8-12 membered bicyclic, or 11-14 membered tricyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, or 11-14 membered tricyclic ring system having one or more heteroatoms (such as O, N, S, P, or Se) and one or more double bonds.

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl. The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, aryl, and heteroaryl mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-β-cyclodextrin or sulfobutyl ether βcyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In some embodiments, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors (Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMKla, CAMK1b, CAMK1d, CAMK1 g, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1 g1, CK1 g2, CK1 g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDR/VEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38 g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120 g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TAO1, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F317I), ABL1(G250E), ABL1(H396P), ABL1(M351T), ABL1(Q252H), ABL1(T315I), ABL1(Y253F), ALK (C1156Y), ALK(L1196M), ALK (F1174L), ALK (R1275Q), BRAF(V599E), BTK(E41K), CHK2(I157T), c-Kit(A829P), c-KIT(D816H), c-KIT(D816V), c-Kit(D820E), c-Kit(N822K), C-Kit (T670I), c-Kit(V559D), c-Kit(V559D/V654A), c-Kit(V559D/T670I), C-Kit (V560G), c-KIT(V654A), C-MET(D1228H), C-MET(D1228N), C-MET(F1200I), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (I2020T), LRRK2 (R1441C), p38a(T106M), PDGFRa(D842V), PDGFRa(T674I), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors (HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), drug-antibody conjugate (e.g brentuximab vedotin, ibritumomab tioxetan), cancer immunotherapy such as Interleukin-2, cancer vaccines (e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, antimetabolites, topoisomerase inhibitors, anti-microtubule agents, kinase inhibitors, epigenetic agents, HSP90 inhibitors, PARP inhibitors, BCL-2 inhibitor, drug-antibody conjugate, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

In certain embodiments, the compounds of the invention are administered in combination with one or more anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates. The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as entanercept, or infliximab, which is an anti-TNF alpha monoclonal antibody.

In certain embodiments, the compounds of the invention are administered in combination with one or more immunosuppressant agents.

In some embodiments, the immunosuppressant agent is glucocorticoid, methotrexate, cyclophosphamide, azathioprine, mercaptopurine, leflunomide, cyclosporine, tacrolimus, and mycophenolate mofetil, dactinomycin, anthracyclines, mitomycin C, bleomycin, or mithramycin, or fingolimod.

The invention further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of reaction schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

An approach to synthesize compounds of

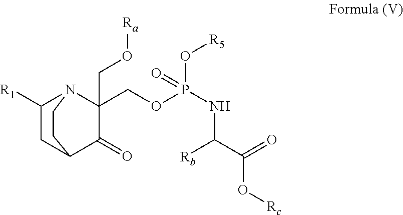

is described in Scheme 1. R₁, R₅, Rₐ, R_{b}, and R_{c} in general Scheme 1 is the same as those described in the Summary section above.

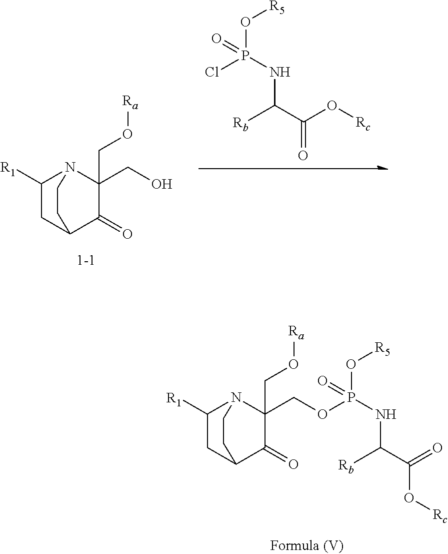

In Scheme 1, the starting material 1-1 prepared by standard organic reaction can react with appropriate phosphorochloridate reagent to form the target compounds. The phosphorochloridate reagent can be prepared by reacting the phosphorodichloridate reagents with the amino analogues.

The Formula (IV) compounds

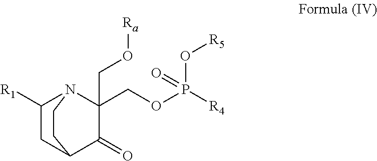

can be made by the method similar to Scheme 1, by using different starting material, intermediates, and reagents.

The compounds

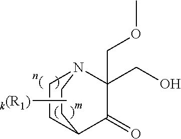

is described in Scheme 2 below. R₁, k, m, and n, in general Scheme 2 is the same as those described in the Summary section above.

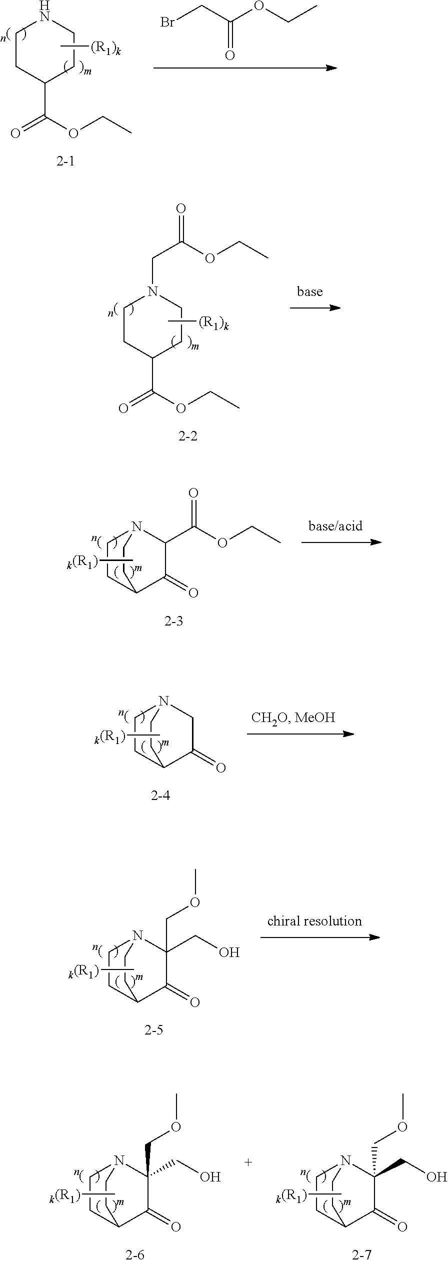

In Scheme 2, the starting material 2-1 is N-alkylated to yield bi-ester 2-2, which is treated with base to form the fused cyclic compound 2-3. Next, the decarboxylation condition generates ketone 2-4, which is treated with CH₂O/MeOH to afford 2-5. Finally, the chiral resolution provides the optically pure compound, exemplified by isomer 2-6 and 2-7.

The Formula (II) compounds

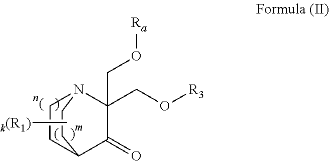

can be made by the method similar to Scheme 2, by using different starting material, intermediates, and reagents.

The Formula (I) compounds

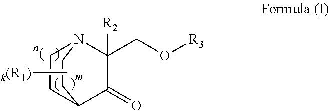

can be made by the method similar to Scheme 2, by using different starting material, intermediates, and reagents.

The compounds

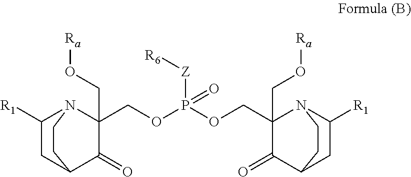

is described in Scheme A below. R₁, R₆, and Rₐ in general Scheme A is the same as those described in the Summary section above.

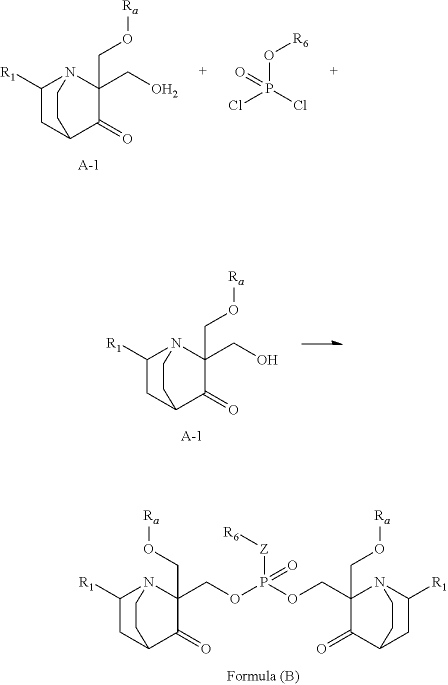

In Scheme A, the starting material A-1 prepared by the method similar to Scheme 2 can react with appropriate phosphorodichloridate reagent to form the target compounds. The phosphorodichloridate reagent can be prepared by reacting the phosphorodichloridate reagents with the amino analogues.

The compounds

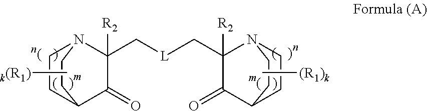

can be made by the method similar to Scheme A, by using different starting material, intermediates, and reagents.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example 1: Preparation of isopropyl (2S)-2-([[2-(methoxymethyl)-3-oxo-1-azabicyclo[2.2.2]octan-2-yl]methoxy(phenoxy)phosphoryl]amino)propanoate

Synthesis of 2-(hydroxymethyl)-2-(methoxymethyl)-1-azabicyclo[2.2.2]octan-3-one: Into a 1000-mL round-bottom flask, was placed. This was followed by the addition of K₂CO₃ (55.21 g, 399.478 mmol, 1.00 equiv) at degrees C., H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. The mixture was dried over anhydrous sodium sulfate and concentrated. The residue was applied onto a silica gel column with ethyl acetate/petroleum ether (0:1-1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. H-NMR: ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of isopropyl (2S)-2-[[chloro(phenoxy)phosphoryl]amino]propanoate: Into a 250-mL 3-necked round-bottom flask, was placed isopropyl (2S)-2-aminopropanoate hydrochloride (11.92 g, 71.097 mmol, 1.0 equiv), DCM (150.00 mL), TEA (17.99 g, 177.742 mmol, 2.50 equiv). This was followed by the addition of phenyl dichlorophosphate (15.00 g, 71.097 mmol, 1.00 equiv) dropwise with stirring at −78 degrees C. The resulting solution was stirred for 3 hr at −78 degrees C. The resulting mixture was concentrated under lower temperature. The residue was dissolved in 100 mL of Et2O. The solids were filtered out. The resulting mixture was concentrated under lower temperature. The residue was applied onto a silica gel column with ethyl acetate/petroleum ether (1:20-1:4). This resulted in 3.9 g (17.94%) of isopropyl (2S)-2-[[chloro(phenoxy)phosphoryl]amino]propanoate as colorless oil. LC-MS: (ES, m/z): [M+1]=306. H-NMR: (300 MHz, Chloroform-d) δ 7.51-7.34 (m, 2H), 7.28 (ddd, J=8.8, 4.6, 2.2 Hz, 3H), 5.20-4.99 (m, J=6.2 Hz, 1H), 4.34 (dt, J=21.9, 10.7 Hz, 1H), 4.15 (m, 1H), 1.52 (dd, J=7.0, 1.7 Hz, 3H), 1.30 (dt, J=6.4, 4.1 Hz, 6H).

Synthesis of isopropyl (2S)-2-([[2-(methoxymethyl)-3-oxo-1-azabicyclo[2.2.2]octan-2-yl]methoxy(phenoxy)phosphoryl]amino)propanoate: Into a 50-mL 3-necked round-bottom flask, was placed 2-(hydroxymethyl)-2-(methoxymethyl)-1-azabicyclo[2.2.2]octan-3-one (100 mg, 1.00 equiv), THE (5 mL), NMI (82.4 mg, 2.0 equiv). This was followed by the addition of a solution of isopropyl (2S)-2-[[chloro(phenoxy)phosphoryl]amino]propanoate (230 mg, 1.5 equiv) in THE (1 mL) dropwise with stirring at 0 degrees C. The resulting solution was stirred for 1 hr at room temperature. The resulting mixture was concentrated under lower temperature. The residue was dissolved in 4 mL of CH₃CN. The crude was purified by Prep-HPLC with the following conditions (IntelFlash-1): Column, C18; mobile phase, CH₃CN:H2O (0.5% CF3COOH); Detector, 220. 20 mg product was obtained. This resulted in 20 mg (8.51%) of isopropyl (2S)-2-([[2-(methoxymethyl)-3-oxo-1-azabicyclo[2.2.2]octan-2-yl]methoxy(phenoxy)phosphoryl]amino)propanoate as colorless oil. LC-MS: (ES, m/z): [M+1]=469. H-NMR: (300 MHz, Chloroform-d) δ 7.34 (t, J=7.8 Hz, 2H), 7.28-7.12 (m, 3H), 5.12-4.90 (m, 1H), 4.78-4.36 (m, 3H), 4.15-3.83 (m, 4H), 3.76-3.46 (m, 2H), 3.31 (d, J=9.3 Hz, 3H), 2.74 (d, J=3.4 Hz, 1H), 2.47-1.97 (m, 4H), 1.42 (dd, J=7.1, 5.2 Hz, 2H), 1.36-1.14 (m, 6H).

### Example 2: Preparation of 2-(hydroxymethyl)-2-(methoxymethyl)-4-methyl-1-azabicyclo[2.2.2]octan-3-one

Synthesis of 1-tert-butyl 4-methyl 4-methylpiperidine-1,4-dicarboxylate: Into a 250-mL 3-necked round-bottom flask purged and maintained with an inert atmosphere of nitrogen, was placed 1-tert-butyl 4-methyl piperidine-1,4-dicarboxylate (6.36 g, 26.140 mmol, 1.00 equiv), THE (50.00 mL). This was followed by the addition of LDA (34.00 mL, 34.000 mmol, 1.30 equiv) dropwise with stirring at −78 degrees C. The mixture was stirred at −78 degrees C. for 45 min. To this was added CH₃I (5.95 g, 41.919 mmol, 1.60 equiv) dropwise with stirring at −78 degrees C. The resulting solution was stirred for 5 hr at room temperature. The reaction was then quenched by the addition of 300 mL of aqueous NH₄Cl. The resulting solution was extracted with 3×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:9). This resulted in 6.12 g (90.98%) of 1-tert-butyl 4-methyl 4-methylpiperidine-1,4-dicarboxylate as yellow oil. H-NMR (300 MHz, Chloroform-d, ppm) δ 3.81-3.73 (m, 2H), 3.72 (s, 3H), 3.00 (ddd, J=13.8, 10.6, 3.1 Hz, 2H), 2.13-2.03 (m, 2H), 1.46 (s, 9H), 1.38 (ddd, J=14.2, 10.6, 4.2 Hz, 2H), 1.22 (s, 3H).

Synthesis of methyl 4-methylpiperidine-4-carboxylate hydrochloride: Into a 250-mL round-bottom flask, was placed 1-tert-butyl 4-methyl 4-methylpiperidine-1,4-dicarboxylate (6.12 g, 23.783 mmol, 1.00 equiv), HCl (gas) in 1,4-dioxane (4 M, 60.00 mL). The resulting solution was stirred for 2 hr at room temperature. The resulting mixture was concentrated under vacuum. This resulted in 4 g (86.84%) of methyl 4-methylpiperidine-4-carboxylate hydrochloride as a white solid. H-NMR (300 MHz, Methanol-d4, ppm) δ 3.77 (s, 3H), 3.37-3.29 (m, 3H), 3.11-2.97 (m, 2H), 2.38-2.25 (m, 2H), 1.72 (ddd, J=15.3, 11.7, 4.1 Hz, 2H), 1.30 (s, 3H).

Synthesis of methyl 1-(2-ethoxy-2-oxoethyl)-4-methylpiperidine-4-carboxylate: Into a 100-mL round-bottom flask, was placed methyl 4-methylpiperidine-4-carboxylate hydrochloride (4.00 g, 20.654 mmol, 1.00 equiv), DMF (40.00 mL), ethyl bromoacetate (3.61 g, 21.617 mmol, 1.05 equiv), K₂CO₃ (3.00 g, 21.707 mmol, 1.05 equiv), TBAB (668.00 mg, 2.072 mmol, 0.10 equiv). The resulting solution was stirred for 5 hr at room temperature. The reaction was then quenched by the addition of 300 mL of water. The resulting solution was extracted with 2×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:1). This resulted in 3.46 g (68.85%) of methyl 1-(2-ethoxy-2-oxoethyl)-4-methylpiperidine-4-carboxylate as yellow oil. H-NMR (300 MHz, Chloroform-d, ppm) δ 4.18 (q, J=7.1 Hz, 2H), 3.70 (s, 3H), 3.17 (s, 2H), 2.84-2.69 (m, 2H), 2.31-2.09 (m, 4H), 1.58 (ddd, J=13.9, 10.5, 3.8 Hz, 2H), 1.27 (t, J=7.1 Hz, 3H), 1.20 (s, 3H).

Synthesis of ethyl 4-methyl-3-oxo-1-azabicyclo[2.2.2]octane-2-carboxylate: Into a 100-mL round-bottom flask, was placed methyl 1-(2-ethoxy-2-oxoethyl)-4-methylpiperidine-4-carboxylate (3.06 g, 12.577 mmol, 1.00 equiv), toluene (40.00 mL, 375.956 mmol, 29.89 equiv), t-BuOK (2.82 g, 25.131 mmol, 2.00 equiv). The resulting solution was stirred for 3 hr at 110 degrees C. in an oil bath. The reaction mixture was cooled to room temperature. The pH value of the solution was adjusted to 6 with HCl (2 mol/L). The resulting mixture was concentrated under vacuum. The residue was applied onto a silica gel column with dichloromethane/methanol (24:1). This resulted in 1.45 g (54.57%) of ethyl 4-methyl-3-oxo-1-azabicyclo[2.2.2]octane-2-carboxylate as yellow oil. LC-MS: (ES, m/z): M+1: 212

Synthesis of 4-methyl-1-azabicyclo[2.2.2]octan-3-one hydrochloride: Into a 100-mL round-bottom flask, was placed ethyl 4-methyl-3-oxo-1-azabicyclo[2.2.2]octane-2-carboxylate (1.45 g, 6.864 mmol, 1.00 equiv), conc. HCl (10.00 mL). The resulting solution was stirred overnight at 100 degrees C. in an oil bath. The reaction mixture was cooled to room temperature. The resulting mixture was concentrated under vacuum. The crude product was purified by re-crystallization from CH₃CN. The solids were collected by filtration. This resulted in 1 g (82.94%) of 4-methyl-1-azabicyclo[2.2.2]octan-3-one hydrochloride as a white solid. H-NMR (300 MHz, Methanol-d₄, ppm) δ 4.06 (t, J=1.3 Hz, 2H), 3.75-3.45 (m, 4H), 2.22 (ddd, J=13.4, 10.7, 5.5 Hz, 2H), 2.13-1.95 (m, 2H), 1.15 (s, 3H).

Synthesis of 2-(hydroxymethyl)-2-(methoxymethyl)-4-methyl-1-azabicyclo[2.2.2]octan-3-one: Into a 100-mL round-bottom flask, was placed 4-methyl-1-azabicyclo[2.2.2]octan-3-one hydrochloride (200.00 mg, 1.139 mmol, 1.00 equiv), MeOH (9.00 mL), H₂O (3.00 mL), HCHO (462.00 mg, 5.693 mmol, 5.00 equiv, 37%), K₂CO₃ (236.00 mg, 1.708 mmol, 1.50 equiv). The resulting solution was stirred for 8 hr at 80 degrees C. in an oil bath. The reaction mixture was cooled to room temperature. The pH value of the solution was adjusted to 12 with aq. NaOH (2 mol/L). The resulting solution was extracted with 3×50 mL of dichloromethane and the organic layers combined. The resulting mixture was washed with 1×200 ml of brine. The mixture was dried over anhydrous sodium sulfate and concentrated under vacuum. The residue was applied onto a silica gel column with dichloromethane/methanol (7:1). The crude product was purified by Prep-HPLC with the following conditions (2 #SHIMADZU (HPLC-01)): Column, XBridge Prep C18 OBD Column, Sum, 19*150 mm; mobile phase, Water (0.05% NH₃.H₂O) and ACN (4% Phase B up to 35% in 7 min); Detector, 220 nm. This resulted in 16 mg (6.59%) of 2-(hydroxymethyl)-2-(methoxymethyl)-4-methyl-1-azabicyclo[2.2.2]octan-3-one as a white solid. LC-MS: (ES, m/z): M+1: 214; H-NMR (300 MHz, DMSO-d₆, ppm): δ 4.57 (t, J=6.0 Hz, 1H), 3.74-3.48 (m, 4H), 3.30-3.22 (m, 2H), 3.20 (s, 3H), 2.75 (dtd, J=13.5, 10.0, 6.6 Hz, 2H), 1.73 (ddd, J=8.4, 5.9, 3.0 Hz, 4H), 0.82 (s, 3H).

### Example 3: Preparation of Synthesis of (S)-2-(hydroxymethyl)-2-(methoxymethyl)-4-methylquinuclidin-3-one (Assumed) and Synthesis of (S)-2-(hydroxymethyl)-2-(methoxymethyl)-4-methylquinuclidin-3-one (Assumed)

The 2-(hydroxymethyl)-2-(methoxymethyl)-4-methyl-1-azabicyclo[2.2.2]octan-3-one (47.00 mg, 0.22 mmol, 1.00 equiv, 95%) was purified by Chiral-Prep-HPLC with the following conditions: Column, Lux Cellulose-4, 100*4.6 mm, 3 um H19-381245; mobile phase A: n-Hexane (0.1% DEA); mobile phase B: Ethanol; Flow rate: 1.0000 mL/min; Gradient: 0% B to 15% B in 6 min; Detector, 220 nm. This resulted in 15 mg of (S)-2-(hydroxymethyl)-2-(methoxymethyl)-4-methylquinuclidin-3-one (Assumed), RT=3.12 min at SHIMADZU LC-20AD-2) as a white solid. LC-MS: (ES, m/z): M+1: 214, H-NMR: 1H NMR (300 MHz, CDCL₃, ppm) δ 3.98 (t, J=12.0 Hz, 1H), 3.74-3.48 (m, 3H), 3.45-3.34 (m, 5H), 3.06-2.95 (m, 3H), 1.97-1.91 (m, 4H), 0.92 (s, 3H).

The 2-(hydroxymethyl)-2-(methoxymethyl)-4-methyl-1-azabicyclo[2.2.2]octan-3-one (47.00 mg, 0.22 mmol, 1.00 equiv, 95%) was purified by Chiral-Prep-HPLC with the following conditions: Column, Lux Cellulose-4, 100*4.6 mm, 3 um H19-381245; mobile phase A: n-Hexane (0.1% DEA); mobile phase B: Ethanol; Flow rate: 1.0000 mL/min; Gradient: 0% B to 15% B in 6 min; Detector, 220 nm. This resulted in 15 mg of (S)-2-(hydroxymethyl)-2-(methoxymethyl)-4-methylquinuclidin-3-one (Assumed), RT=4.02 min at SHIMADZU LC-20AD-2) as a white solid. LC-MS: (ES, m/z): M+1: 214, H-NMR: 1H NMR (300 MHz, CDCL₃, ppm) δ 3.98 (t, J=12.0 Hz, 1H), 3.74-3.48 (m, 3H), 3.45-3.34 (m, 5H), 3.06-2.95 (m, 3H), 1.97-1.91 (m, 4H), 0.92 (s, 3H).

### Example 4: Preparation of 2,2-bis(hydroxymethyl)-4-methyl-1-azabicyclo[2.2.2]octan-3-one

Synthesis of 1-tert-butyl 4-methyl 4-methylpiperidine-1,4-dicarboxylate: Into a 10000-mL 3-necked round-bottom flask purged and maintained with an inert atmosphere of nitrogen, was placed 1-tert-butyl 4-methyl piperidine-1,4-dicarboxylate (400.0 g, 1.65 mol, 1.00 equiv), THE (4000.00 mL). This was followed by the addition of LDA (2150 mL, 2.15 mol, 1.30 equiv) dropwise with stirring at −78 degrees C. The mixture was stirred at −78 degrees C. for 45 min. To this was added CH₃I (369.0 g, 2.6 mol, 1.60 equiv) dropwise with stirring at −78 degrees C. The resulting solution was stirred for 5 hr at room temperature. The reaction was then quenched by the addition of 3000 mL of aqueous NH₄Cl. The resulting solution was extracted with 3×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:9). This resulted in 400 g (94.5%) of 1-tert-butyl 4-methyl 4-methylpiperidine-1,4-dicarboxylate as yellow oil. H-NMR (300 MHz, Chloroform-d, ppm) δ 3.81-3.73 (m, 2H), 3.72 (s, 3H), 3.00 (ddd, J=13.8, 10.6, 3.1 Hz, 2H), 2.13-2.03 (m, 2H), 1.46 (s, 9H), 1.38 (ddd, J=14.2, 10.6, 4.2 Hz, 2H), 1.22 (s, 3H).

Synthesis of methyl 4-methylpiperidine-4-carboxylate hydrochloride: Into a 5000-mL round-bottom flask, was placed 1-tert-butyl 4-methyl 4-methylpiperidine-1,4-dicarboxylate (380 g, 1.48 mol, 1.00 equiv), HCl (gas) in 1,4-dioxane (4 M, 3000 mL). The resulting solution was stirred for 6 hr at room temperature. The resulting mixture was concentrated under vacuum. This resulted in 251 g (88%) of methyl 4-methylpiperidine-4-carboxylate hydrochloride as a white solid. H-NMR (300 MHz, Methanol-d₄, ppm) δ 3.77 (s, 3H), 3.37-3.29 (m, 3H), 3.11-2.97 (m, 2H), 2.38-2.25 (m, 2H), 1.72 (ddd, J=15.3, 11.7, 4.1 Hz, 2H), 1.30 (s, 3H).

Synthesis of methyl 1-(2-ethoxy-2-oxoethyl)-4-methylpiperidine-4-carboxylate: Into a 100-mL round-bottom flask, was placed methyl 4-methylpiperidine-4-carboxylate hydrochloride (250 g, 1.3 mol, 1.00 equiv), DMF (2500 mL), ethyl bromoacetate (229 g, 1.37 mmol, 1.05 equiv), K₂CO₃ (189.0 g, 1.37 mol, 1.05 equiv), TBAB (42 g, 0.13 mol, 0.10 equiv).

The resulting solution was stirred for 5 hr at room temperature. The reaction was then quenched by the addition of 1000 mL of water. The resulting solution was extracted with 2×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:1). This resulted in 220 g (70.0%) of methyl 1-(2-ethoxy-2-oxoethyl)-4-methylpiperidine-4-carboxylate as yellow oil. H-NMR (300 MHz, Chloroform-d, ppm) δ 4.18 (q, J=7.1 Hz, 2H), 3.70 (s, 3H), 3.17 (s, 2H), 2.84-2.69 (m, 2H), 2.31-2.09 (m, 4H), 1.58 (ddd, J=13.9, 10.5, 3.8 Hz, 2H), 1.27 (t, J=7.1 Hz, 3H), 1.20 (s, 3H).

Synthesis of ethyl 4-methyl-3-oxo-1-azabicyclo[2.2.2]octane-2-carboxylate: Into a 3000-mL round-bottom flask, was placed methyl 1-(2-ethoxy-2-oxoethyl)-4-methylpiperidine-4-carboxylate (200 g, 0.82 mol, 1.00 equiv), toluene (1000 mL), t-BuOK (188 g, 1.64 mol, 2.00 equiv). The resulting solution was stirred for 3 hr at 110 degrees C. in an oil bath. The reaction mixture was cooled to room temperature. The pH value of the solution was adjusted to 6 with HCl (2 mol/L). The resulting mixture was concentrated under vacuum. The residue was applied onto a silica gel column with dichloromethane/methanol (24:1). This resulted in 121 g (70.0%) of ethyl 4-methyl-3-oxo-1-azabicyclo[2.2.2]octane-2-carboxylate as yellow oil. LC-MS: (ES, m/z): M+1: 212

Synthesis of 4-methyl-1-azabicyclo[2.2.2]octan-3-one hydrochloride: Into a 100-mL round-bottom flask, was placed ethyl 4-methyl-3-oxo-1-azabicyclo[2.2.2]octane-2-carboxylate (120 g, 6.864 mmol, 1.00 equiv), conc. HCl (10.00 mL). The resulting solution was stirred overnight at 100 degrees C. in an oil bath. The reaction mixture was cooled to room temperature. The resulting mixture was concentrated under vacuum. The crude product was purified by re-crystallization from CH₃CN. The solids were collected by filtration. This resulted in 89 g (90.0%) of 4-methyl-1-azabicyclo[2.2.2]octan-3-one hydrochloride as a white solid. H-NMR (300 MHz, Methanol-d₄, ppm) δ 4.06 (t, J=1.3 Hz, 2H), 3.75-3.45 (m, 4H), 2.22 (ddd, J=13.4, 10.7, 5.5 Hz, 2H), 2.13-1.95 (m, 2H), 1.15 (s, 3H).

Synthesis of 2,2-bis(hydroxymethyl)-4-methyl-1-azabicyclo[2.2.2]octan-3-one: Into a 250-mL round-bottom flask, was placed 4-methyl-1-azabicyclo[2.2.2]octan-3-one hydrochloride (5.00 g, 28.464 mmol, 1.00 equiv), HCHO (46.33 g, 570.909 mmol, 20.06 equiv, 37%), K₂CO₃ (4.73 g, 34.224 mmol, 1.20 equiv). The resulting solution was stirred for 1 hr at 55 degrees C. in an oil bath. The reaction mixture was cooled to room temperature. The resulting solution was diluted with 100 mL of water. The resulting solution was extracted with 3×100 mL of dichloromethane and the organic layers combined and dried over anhydrous sodium sulfate and concentrated under vacuum. The residue was applied onto a silica gel column with dichloromethane/methanol (5:1). The crude product was purified by re-crystallization from Et₂O. The solids were collected by filtration. This resulted in 870 mg (15.34%) of 2,2-bis(hydroxymethyl)-4-methyl-1-azabicyclo[2.2.2]octan-3-one as a white solid. LC-MS: (ES, m/z): M+1: 200; H-NMR (300 MHz, DMSO-d₆, ppm) δ 4.60 (t, J=5.8 Hz, 2H), 3.73 (dd, J=11.6, 5.5 Hz, 2H), 3.62 (dd, J=11.6, 6.0 Hz, 2H), 3.39-3.34 (m, 1H), 3.31-3.26 (m, 1H), 2.91-2.68 (m, 2H), 1.82-1.69 (m, 4H), 0.83 (s, 3H).

### Example 5: Preparation of 2-(hydroxymethyl)-4-isopropyl-2-(methoxymethyl)-1-azabicyclo[2.2.2]octan-3-one

Synthesis of 1-tert-butyl 4-methyl 4-isopropylpiperidine-1,4-dicarboxylate: Into a 250-mL 3-necked round-bottom flask purged and maintained with an inert atmosphere of nitrogen, was placed 1-tert-butyl 4-methyl piperidine-1,4-dicarboxylate (9.72 g, 39.950 mmol, 1.00 equiv), THE (97.00 mL). This was followed by the addition of LiHMDS (52.00 mL, 52.000 mmol, 1.30 equiv) dropwise with stirring at −78 degrees C. stirring at −78 degrees C. for 30 min. To this was added 2-iodopropane (10.87 g, 63.944 mmol, 1.60 equiv) dropwise with stirring at −78 degrees C. The resulting solution was stirred overnight at room temperature. The reaction was then quenched by the addition of 200 mL of NH₄Cl. The resulting solution was extracted with 3×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:5). This resulted in 9 g (78.94%) of 1-tert-butyl 4-methyl 4-isopropylpiperidine-1,4-dicarboxylate as a white solid. LC-MS: (ES, m/z): M-56+CH₃CN=271

Synthesis of methyl 4-isopropylpiperidine-4-carboxylate hydrochloride: Into a 100-mL round-bottom flask, was placed 1-tert-butyl 4-methyl 4-isopropylpiperidine-1,4-dicarboxylate (9.00 g, 31.536 mmol, 1.00 equiv), HCl (g) in dioxane (4M, 50.00 mL). The resulting solution was stirred for 3 h at room temperature. The resulting mixture was concentrated. This resulted in 8 g (crude) of methyl 4-isopropylpiperidine-4-carboxylate hydrochloride as a white solid. LC-MS: (ES, m/z): M-HCl+1=186.

Synthesis of methyl 1-(2-ethoxy-2-oxoethyl)-4-isopropylpiperidine-4-carboxylate: Into a 250-mL round-bottom flask, was placed methyl 4-isopropylpiperidine-4-carboxylate hydrochloride (7.20 g, 32.472 mmol, 1.00 equiv), DMF (100.00 mL), ethyl bromoacetate (5.68 g, 34.012 mmol, 1.05 equiv), K₂CO₃ (4.71 g, 34.080 mmol, 1.05 equiv), TBAB (1.06 g, 3.288 mmol, 0.10 equiv). The resulting solution was stirred overnight at room temperature.

The reaction was then quenched by the addition of 500 mL of water. The resulting solution was extracted with 3×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:1.5). This resulted in 6.4 g (72.63%) of methyl 1-(2-ethoxy-2-oxoethyl)-4-isopropylpiperidine-4-carboxylate as light yellow oil. H-NMR (300 MHz, DMSO-d₆, ppm) δ 4.06 (q, J=7.1 Hz, 2H), 3.62 (s, 3H), 3.12 (s, 2H), 2.73 (dt, J=11.8, 3.4 Hz, 2H), 2.12-1.92 (m, 4H), 1.66 (p, J=6.9 Hz, 1H), 1.41 (td, J=12.6, 4.1 Hz, 2H), 1.17 (t, J=7.1 Hz, 3H), 0.82 (d, J=6.9 Hz, 6H).

Synthesis of ethyl 4-isopropyl-3-oxo-1-azabicyclo[2.2.2]octane-2-carboxylate: Into a 100-mL round-bottom flask, was placed t-BuOK (3.97 g, 35.379 mmol, 1.50 equiv), toluene (40.00 mL), the resulting solution was stirred for 30 min at 110 degrees C. in an oil bath. Then methyl 1-(2-ethoxy-2-oxoethyl)-4-isopropylpiperidine-4-carboxylate (6.40 g, 23.585 mmol, 1.00 equiv) in toluene (10 mL) was added. The resulting solution was stirred for 3 hr at 110 degrees C. in an oil bath. The reaction mixture was cooled to room temperature. The pH value of the solution was adjusted to 6 with HCl (6 mol/L). The resulting mixture was concentrated under vacuum. The residue was applied onto a silica gel column with dichloromethane/methanol (15:1). This resulted in 3.7 g (65.55%) of ethyl 4-isopropyl-3-oxo-1-azabicyclo[2.2.2]octane-2-carboxylate as yellow oil. LC-MS: (ES, m/z): M+1: 240.

Synthesis of 4-isopropyl-1-azabicyclo[2.2.2]octan-3-one hydrochloride: Into a 100-mL round-bottom flask, was placed ethyl 4-isopropyl-3-oxo-1-azabicyclo[2.2.2]octane-2-carboxylate (3.70 g, 15.461 mmol, 1.00 equiv), 6N HCl (40.00 mL). The resulting solution was stirred overnight at 100 degrees C. in an oil bath. The reaction mixture was cooled to room temperature. The resulting mixture was concentrated under vacuum. The crude product was purified by re-crystallization from EtOAc. This resulted in 2.8 g (88.90%) of 4-isopropyl-1-azabicyclo[2.2.2]octan-3-one hydrochloride as a white solid. H-NMR (300 MHz, Methanol-d₄, ppm) δ 4.00 (t, J=1.3 Hz, 2H), 3.71-3.44 (m, 4H), 2.33-1.93 (m, 5H), 0.97 (d, J=6.9 Hz, 6H).

Synthesis of 2-(hydroxymethyl)-4-isopropyl-2-(methoxymethyl)-1-azabicyclo[2.2.2]octan-3-one: Into a 40-mL round-bottom flask, was placed 4-isopropyl-1-azabicyclo[2.2.2]octan-3-one hydrochloride (400.00 mg, 1.964 mmol, 1.00 equiv), MeOH (15.00 mL), H₂O (5.00 mL), K₂CO₃ (816.00 mg, 5.904 mmol, 3.01 equiv), HCHO (1.60 g, 19.716 mmol, 10.04 equiv, 37%). The resulting solution was stirred for 7 hr at 70 degrees C. in an oil bath. The reaction mixture was cooled to room temperature. The reaction was diluted with 200 mL of water. The resulting solution was extracted with 2×100 mL of dichloromethane and the organic layers combined. The resulting mixture was washed with 1×200 ml of brine. The mixture was dried over anhydrous sodium sulfate and concentrated under vacuum. The residue was applied onto a silica gel column with dichloromethane/methanol (20:1). The crude product was purified by Prep-HPLC with the following conditions (2 #SHIMADZU (HPLC-01)): Column, XBridge Prep C18 OBD Column, 5 um, 19*150 mm; mobile phase, Water (10 MMOL/L NH₄HCO₃+0.1% NH₃.H₂O) and ACN (28% Phase B up to 45% in 7 min); Detector, 220 nm. This resulted in 12 mg (2.53%) of 2-(hydroxymethyl)-4-isopropyl-2-(methoxymethyl)-1-azabicyclo[2.2.2]octan-3-one as a white solid. LC-MS: (ES, m/z): M+1: 242; H-NMR (300 MHz, DMSO-d₆, ppm) δ 4.50 (t, J=6.0 Hz, 1H), 3.73-3.50 (m, 4H), 3.31-3.22 (m, 2H), 3.20 (s, 3H), 2.82-2.68 (m, 2H), 1.94-1.52 (m, 5H), 0.78 (d, J=6.9 Hz, 6H).

### Example 6: Preparation of (2S)-4-ethyl-2-(hydroxymethyl)-2-(methoxymethyl)-1-azabicyclo[2.2.2]octan-3-one (Assumed) and (2R)-4-ethyl-2-(hydroxymethyl)-2-(methoxymethyl)-1-azabicyclo[2.2.2]octan-3-one (Assumed)

Synthesis of 1-tert-butyl 4-methyl 4-ethylpiperidine-1,4-dicarboxylate: Into a 250-mL 3-necked round-bottom flask purged and maintained with an inert atmosphere of nitrogen, was placed 1-tert-butyl 4-methyl piperidine-1,4-dicarboxylate (10.00 g, 41.101 mmol, 1.00 equiv), THE (100.00 mL). This was followed by the addition of LDA (61.65 mL, 61.650 mmol, 1.50 equiv) dropwise with stirring at −78 degrees C. The resulting solution was stirred for 45 min at −78 degrees C. To this was added ethyl iodide (10.23 g, 65.591 mmol, 1.60 equiv) dropwise with stirring at −78 degrees C. The resulting solution was stirred overnight at room temperature. The reaction was then quenched by the addition of 500 mL of NH₄Cl. The resulting solution was extracted with 2×300 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:10). This resulted in 10.66 g (95.58%) of 1-tert-butyl 4-methyl 4-ethylpiperidine-1,4-dicarboxylate as yellow oil. H-NMR (300 MHz, Chloroform-d, ppm) δ 3.88 (dt, J=13.9, 4.0 Hz, 2H), 3.72 (s, 3H), 2.88 (ddd, J=14.1, 11.7, 2.8 Hz, 2H), 2.18-2.03 (m, 2H), 1.57 (q, J=7.5 Hz, 2H), 1.46 (s, 9H), 1.34 (ddd, J=13.5, 11.6, 4.4 Hz, 2H), 0.83 (t, J=7.5 Hz, 3H).

Synthesis of methyl 4-ethylpiperidine-4-carboxylate hydrochloride: Into a 250-mL round-bottom flask, was placed 1-tert-butyl 4-methyl 4-ethylpiperidine-1,4-dicarboxylate (10.66 g, 39.284 mmol, 1.00 equiv), HCl (gas) in 1,4-dioxane (4M, 100.00 mL). The resulting solution was stirred for 2 hr at room temperature. The resulting mixture was concentrated under vacuum. The crude product was purified by re-crystallization from EtOAc. The solids were collected by filtration. This resulted in 7.5 g (91.92%) of methyl 4-ethylpiperidine-4-carboxylate hydrochloride as a white solid. H-NMR (300 MHz, Methanol-d₄, ppm) δ 3.77 (s, 3H), 3.39-3.30 (m, 2H), 3.07-2.89 (m, 2H), 2.33 (dq, J=14.8, 3.0 Hz, 2H), 1.76-1.60 (m, 4H), 0.86 (t, J=7.5 Hz, 3H).

Synthesis of methyl 1-(2-ethoxy-2-oxoethyl)-4-ethylpiperidine-4-carboxylate: Into a 250-mL round-bottom flask, was placed methyl 4-ethylpiperidine-4-carboxylate hydrochloride (7.50 g, 36.110 mmol, 1.00 equiv), DMF (80.00 mL), ethyl bromoacetate (6.32 g, 37.844 mmol, 1.05 equiv), K₂CO₃ (5.25 g, 37.987 mmol, 1.05 equiv), TBAB (1.22 g, 3.784 mmol, 0.10 equiv). The resulting solution was stirred overnight at room temperature. The reaction was then quenched by the addition of 500 mL of water. The resulting solution was extracted with 2×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 7.9 g (85.02%) of methyl 1-(2-ethoxy-2-oxoethyl)-4-ethylpiperidine-4-carboxylate as yellow oil. H-NMR (300 MHz, Chloroform-d, ppm) δ 4.19 (q, J=7.1 Hz, 2H), 3.71 (s, 3H), 3.17 (s, 2H), 2.94-2.75 (m, 2H), 2.26-2.10 (m, 4H), 1.63-1.51 (m, 4H), 1.28 (t, J=7.1 Hz, 3H), 0.81 (t, J=7.5 Hz, 3H).

Synthesis of ethyl 4-ethyl-3-oxo-1-azabicyclo[2.2.2]octane-2-carboxylate: Into a 250-mL round-bottom flask purged and maintained with an inert atmosphere of nitrogen, was placed t-BuOK (5.16 g, 45.984 mmol, 1.50 equiv), toluene (80.00 mL, 751.912 mmol, 24.49 equiv), the resulting solution was stirred for 30 min at 110 degrees C. in an oil bath. Then methyl 1-(2-ethoxy-2-oxoethyl)-4-ethylpiperidine-4-carboxylate (7.90 g, 30.700 mmol, 1.00 equiv) in toluene (10 mL) was added. The resulting solution was stirred for 3 hr at 110 degrees C. in an oil bath. The reaction mixture was cooled to room temperature. The pH value of the solution was adjusted to 6 with HCl (6 mol/L). The resulting mixture was concentrated under vacuum. The residue was applied onto a silica gel column with dichloromethane/methanol (15:1). This resulted in 6.5 g (93.98%) of ethyl 4-ethyl-3-oxo-1-azabicyclo[2.2.2]octane-2-carboxylate as yellow oil. LC-MS: (ES, m/z): M+1: 226 Synthesis of 4-ethyl-1-azabicyclo[2.2.2]octan-3-one hydrochloride: Into a 100-mL round-bottom flask, was placed ethyl 4-ethyl-3-oxo-1-azabicyclo[2.2.2]octane-2-carboxylate (6.50 g, 28.852 mmol, 1.00 equiv), HCl (6N, 60.00 mL). The resulting solution was stirred overnight at 100 degrees C. in an oil bath. The reaction mixture was cooled to room temperature. The resulting mixture was concentrated under vacuum. The crude product was purified by re-crystallization from EtOAc. The solids were collected by filtration. This resulted in 4 g (73.09%) of 4-ethyl-1-azabicyclo[2.2.2]octan-3-one hydrochloride as a brown solid. H-NMR (300 MHz, Methanol-d₄, ppm) δ 4.03 (t, J=1.3 Hz, 2H), 3.74-3.45 (m, 4H), 2.21 (ddd, J=13.4, 10.7, 5.5 Hz, 2H), 2.08-1.94 (m, 2H), 1.63 (q, J=7.6 Hz, 2H), 0.95 (t, J=7.5 Hz, 3H).

Synthesis of (2S)-4-ethyl-2-(hydroxymethyl)-2-(methoxymethyl)-1-azabicyclo[2.2.2]octan-3-one (Assumed) and (2R)-4-ethyl-2-(hydroxymethyl)-2-(methoxymethyl)-1-azabicyclo[2.2.2]octan-3-one (Assumed): Into a 100-mL round-bottom flask, was placed 4-ethyl-1-azabicyclo[2.2.2]octan-3-one hydrochloride (1.00 g, 5.272 mmol, 1.00 equiv), MeOH (24.00 mL), H₂O (8.00 mL), K₂CO₃ (2.19 g, 15.846 mmol, 3.01 equiv), HCHO (4.29 g, 52.864 mmol, 10.03 equiv, 37%). The resulting solution was stirred for 5 hr at 70 degrees C. in an oil bath. The reaction mixture was cooled to room temperature. The resulting mixture was concentrated under vacuum. The residue was applied onto a silica gel column with dichloromethane/methanol (20:1). The crude product was purified by Prep-HPLC with the following conditions (2 #SHIMADZU (HPLC-01)): Column, X Bridge Prep C18 OBD Column, Sum, 19*150 mm; mobile phase, Water (0.05% NH₃.H₂O) and ACN (15% Phase B up to 50% in 7 min); Detector, 220 nm. This resulted in 80 mg (6.68%) of 4-ethyl-2-(hydroxymethyl)-2-(methoxymethyl)-1-azabicyclo[2.2.2]octan-3-one as a white solid. The compound 4-ethyl-2-(hydroxymethyl)-2-(methoxymethyl)-1-azabicyclo[2.2.2]octan-3-one (70.00 mg, 0.308 mmol, 1.00 equiv) was purified by Chiral-Prep-HPLC with the following conditions (XA-Prep Chiral HPLC-02): Column, Lux 5u Cellulose-4, AXIA Packed, 2.12*25 cm, 5 um; mobile phase, Hex- and EtOH- (hold 50% EtOH in 25 min); Detector, UV. This resulted in 25 mg (35.71%) of (2S)-4-ethyl-2-(hydroxymethyl)-2-(methoxymethyl)-1-azabicyclo[2.2.2]octan-3-one (assumed) as a white solid. LC-MS (ES, m/z): M+1: 228; H-NMR: (300 MHz, DMSO-d₆, ppm) δ 4.52 (s, 1H), 3.72-3.49 (m, 4H), 3.30-3.22 (m, 2H), 3.20 (s, 3H), 2.86-2.66 (m, 2H), 1.84-1.54 (m, 4H), 1.29 (q, J=7.5 Hz, 2H), 0.76 (t, J=7.5 Hz, 3H). And 25 mg (35.71%) of (2R)-4-ethyl-2-(hydroxymethyl)-2-(methoxymethyl)-1-azabicyclo[2.2.2]octan-3-one (assumed) as a white solid. LC-MS (ES, m/z): M+1: 228; H-NMR (300 MHz, DMSO-d₆, ppm) δ 4.52 (s, 1H), 3.72-3.49 (m, 4H), 3.30-3.22 (m, 2H), 3.20 (s, 3H), 2.86-2.66 (m, 2H), 1.84-1.54 (m, 4H), 1.29 (q, J=7.5 Hz, 2H), 0.76 (t, J=7.5 Hz, 3H).

### Example 7: Preparation of (2S)-2-(ethoxymethyl)-2-(hydroxymethyl)-4-methyl-1-azabicyclo[2.2.2]octan-3-one (Assumed) and (2R)-2-(ethoxymethyl)-2-(hydroxymethyl)-4-methyl-1-azabicyclo[2.2.2]octan-3-one (Assumed)

Synthesis of 1-tert-butyl 4-methyl 4-methylpiperidine-1,4-dicarboxylate: Into a 10000-mL 3-necked round-bottom flask purged and maintained with an inert atmosphere of nitrogen, was placed 1-tert-butyl 4-methyl piperidine-1,4-dicarboxylate (400.0 g, 1.65 mol, 1.00 equiv), THE (4000.00 mL). This was followed by the addition of LDA (2150 mL, 2.15 mol, 1.30 equiv) dropwise with stirring at −78 degrees C. The mixture was stirred at −78 degrees C. for 45 min. To this was added CH₃I (369.0 g, 2.6 mol, 1.60 equiv) dropwise with stirring at −78 degrees C. The resulting solution was stirred for 5 hr at room temperature. The reaction was then quenched by the addition of 3000 mL of aqueous NH₄Cl. The resulting solution was extracted with 3×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:9). This resulted in 400 g (94.5%) of 1-tert-butyl 4-methyl 4-methylpiperidine-1,4-dicarboxylate as yellow oil. H-NMR (300 MHz, Chloroform-d, ppm) δ 3.81-3.73 (m, 2H), 3.72 (s, 3H), 3.00 (ddd, J=13.8, 10.6, 3.1 Hz, 2H), 2.13-2.03 (m, 2H), 1.46 (s, 9H), 1.38 (ddd, J=14.2, 10.6, 4.2 Hz, 2H), 1.22 (s, 3H).

Synthesis of methyl 4-methylpiperidine-4-carboxylate hydrochloride: Into a 5000-mL round-bottom flask, was placed 1-tert-butyl 4-methyl 4-methylpiperidine-1,4-dicarboxylate (380 g, 1.48 mol, 1.00 equiv), HCl (gas) in 1,4-dioxane (4 M, 3000 mL). The resulting solution was stirred for 6 hr at room temperature. The resulting mixture was concentrated under vacuum. This resulted in 251 g (88%) of methyl 4-methylpiperidine-4-carboxylate hydrochloride as a white solid. H-NMR (300 MHz, Methanol-d₄, ppm) δ 3.77 (s, 3H), 3.37-3.29 (m, 3H), 3.11-2.97 (m, 2H), 2.38-2.25 (m, 2H), 1.72 (ddd, J=15.3, 11.7, 4.1 Hz, 2H), 1.30 (s, 3H).

Synthesis of methyl 1-(2-ethoxy-2-oxoethyl)-4-methylpiperidine-4-carboxylate: Into a 100-mL round-bottom flask, was placed methyl 4-methylpiperidine-4-carboxylate hydrochloride (250 g, 1.3 mol, 1.00 equiv), DMF (2500 mL), ethyl bromoacetate (229 g, 1.37 mmol, 1.05 equiv), K₂CO₃ (189.0 g, 1.37 mol, 1.05 equiv), TBAB (42 g, 0.13 mol, 0.10 equiv). The resulting solution was stirred for 5 hr at room temperature. The reaction was then quenched by the addition of 1000 mL of water. The resulting solution was extracted with 2×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:1). This resulted in 220 g (70.0%) of methyl 1-(2-ethoxy-2-oxoethyl)-4-methylpiperidine-4-carboxylate as yellow oil. H-NMR (300 MHz, Chloroform-d, ppm) δ 4.18 (q, J=7.1 Hz, 2H), 3.70 (s, 3H), 3.17 (s, 2H), 2.84-2.69 (m, 2H), 2.31-2.09 (m, 4H), 1.58 (ddd, J=13.9, 10.5, 3.8 Hz, 2H), 1.27 (t, J=7.1 Hz, 3H), 1.20 (s, 3H).

Synthesis of ethyl 4-methyl-3-oxo-1-azabicyclo[2.2.2]octane-2-carboxylate: Into a 3000-mL round-bottom flask, was placed methyl 1-(2-ethoxy-2-oxoethyl)-4-methylpiperidine-4-carboxylate (200 g, 0.82 mol, 1.00 equiv), toluene (1000 mL), t-BuOK (188 g, 1.64 mol, 2.00 equiv). The resulting solution was stirred for 3 hr at 110 degrees C. in an oil bath. The reaction mixture was cooled to room temperature. The pH value of the solution was adjusted to 6 with HCl (2 mol/L). The resulting mixture was concentrated under vacuum. The residue was applied onto a silica gel column with dichloromethane/methanol (24:1). This resulted in 121 g (70.0%) of ethyl 4-methyl-3-oxo-1-azabicyclo[2.2.2]octane-2-carboxylate as yellow oil. LC-MS: (ES, m/z): M+1: 212

Synthesis of 4-methyl-1-azabicyclo[2.2.2]octan-3-one hydrochloride: Into a 100-mL round-bottom flask, was placed ethyl 4-methyl-3-oxo-1-azabicyclo[2.2.2]octane-2-carboxylate (120 g, 6.864 mmol, 1.00 equiv), conc. HCl (10.00 mL). The resulting solution was stirred overnight at 100 degrees C. in an oil bath. The reaction mixture was cooled to room temperature. The resulting mixture was concentrated under vacuum. The crude product was purified by re-crystallization from CH₃CN. The solids were collected by filtration. This resulted in 89 g (90.0%) of 4-methyl-1-azabicyclo[2.2.2]octan-3-one hydrochloride as a white solid. H-NMR (300 MHz, Methanol-d₄, ppm) δ 4.06 (t, J=1.3 Hz, 2H), 3.75-3.45 (m, 4H), 2.22 (ddd, J=13.4, 10.7, 5.5 Hz, 2H), 2.13-1.95 (m, 2H), 1.15 (s, 3H).

Synthesis of (2S)-2-(ethoxymethyl)-2-(hydroxymethyl)-4-methyl-1-azabicyclo[2.2.2]octan-3-one (Assumed) and (2R)-2-(ethoxymethyl)-2-(hydroxymethyl)-4-methyl-1-azabicyclo[2.2.2]octan-3-one (Assumed): Into a 100-mL round-bottom flask purged and maintained with an inert atmosphere of nitrogen, was placed 4-methyl-1-azabicyclo[2.2.2]octan-3-one hydrochloride (1.00 g, 5.693 mmol, 1.00 equiv), EtOH (15.00 mL), H₂O (5.00 mL), K₂CO₃ (3.95 g, 28.581 mmol, 5.02 equiv), HCHO (4.64 g, 57.177 mmol, 10.04 equiv, 37%). The resulting solution was stirred for 6 hr at 70 degrees C. in an oil bath. The reaction mixture was cooled to room temperature. The resulting solution was diluted with 200 mL of water. The resulting solution was extracted with 3×100 mL of dichloromethane and the organic layers combined. The resulting mixture was washed with 1×500 ml of brine. The mixture was dried over anhydrous sodium sulfate and concentrated under vacuum. The residue was applied onto a silica gel column with dichloromethane/methanol (30:1). The crude product was purified by Prep-HPLC with the following conditions (2 #SHIMADZU (HPLC-01)): Column, Sunfire Prep C18 OBD Column, 50*250 mm 5 um 10 nm; mobile phase, Water (0.05% NH₃.H₂O) and MeOH:ACN=1:1 (10% Phase B up to 40% in 15 min); Detector, UV. The crude product was purified by Chiral-Prep-HPLC with the following conditions (XA-Prep Chiral HPLC-02): Column, Lux 5u Cellulose-4, AXIA Packed, 2.12*25 cm, 5 um; mobile phase, Hex- and EtOH- (hold 50% EtOH- in 25 min); Detector, UV. This resulted in 25 mg (1.93%) of (2S)-2-(ethoxymethyl)-2-(hydroxymethyl)-4-methyl-1-azabicyclo[2.2.2]octan-3-one (assumed) as a white solid and 20 mg (1.55%) of (2R)-2-(ethoxymethyl)-2-(hydroxymethyl)-4-methyl-1-azabicyclo[2.2.2]octan-3-one (assumed) as a white solid. LC-MS (ES, m/z): M+1: 228; ¹H NMR (300 MHz, DMSO-d₆, ppm) δ 4.52 (s, 1H), 3.68 (d, J=10.3 Hz, 2H), 3.62-3.48 (m, 2H), 3.38 (q, J=7.0 Hz, 2H), 3.28 (t, J=6.7 Hz, 2H), 2.74 (ddd, J=19.5, 13.5, 7.6 Hz, 2H), 1.81-1.62 (m, 4H), 1.05 (t, J=7.0 Hz, 3H), 0.82 (s, 3H). LC-MS (ES, m/z): M+1: 228; ¹H NMR (300 MHz, DMSO-d₆, ppm) δ 4.52 (s, 1H), 3.68 (d, J=10.3 Hz, 2H), 3.62-3.48 (m, 2H), 3.38 (q, J=7.0 Hz, 2H), 3.28 (t, J=6.7 Hz, 2H), 2.74 (ddd, J=19.5, 13.5, 7.6 Hz, 2H), 1.81-1.62 (m, 4H), 1.05 (t, J=7.0 Hz, 3H), 0.82 (s, 3H).

### Example 8: Preparation of 2-(hydroxymethyl)-2-(isopropoxymethyl)-4-methyl-1-azabicyclo[2.2.2]octan-3-one

Synthesis of 1-tert-butyl 4-methyl 4-methylpiperidine-1,4-dicarboxylate: Into a 10000-mL 3-necked round-bottom flask purged and maintained with an inert atmosphere of nitrogen, was placed 1-tert-butyl 4-methyl piperidine-1,4-dicarboxylate (400.0 g, 1.65 mol, 1.00 equiv), THE (4000.00 mL). This was followed by the addition of LDA (2150 mL, 2.15 mol, 1.30 equiv) dropwise with stirring at −78 degrees C. The mixture was stirred at −78 degrees C. for 45 min. To this was added CH₃I (369.0 g, 2.6 mol, 1.60 equiv) dropwise with stirring at −78 degrees C. The resulting solution was stirred for 5 hr at room temperature. The reaction was then quenched by the addition of 3000 mL of aqueous NH₄Cl. The resulting solution was extracted with 3×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:9). This resulted in 400 g (94.5%) of 1-tert-butyl 4-methyl 4-methylpiperidine-1,4-dicarboxylate as yellow oil. H-NMR (300 MHz, Chloroform-d, ppm) δ 3.81-3.73 (m, 2H), 3.72 (s, 3H), 3.00 (ddd, J=13.8, 10.6, 3.1 Hz, 2H), 2.13-2.03 (m, 2H), 1.46 (s, 9H), 1.38 (ddd, J=14.2, 10.6, 4.2 Hz, 2H), 1.22 (s, 3H).

Synthesis of methyl 4-methylpiperidine-4-carboxylate hydrochloride: Into a 5000-mL round-bottom flask, was placed 1-tert-butyl 4-methyl 4-methylpiperidine-1,4-dicarboxylate (380 g, 1.48 mol, 1.00 equiv), HCl (gas) in 1,4-dioxane (4 M, 3000 mL). The resulting solution was stirred for 6 hr at room temperature. The resulting mixture was concentrated under vacuum. This resulted in 251 g (88%) of methyl 4-methylpiperidine-4-carboxylate hydrochloride as a white solid. H-NMR (300 MHz, Methanol-d₄, ppm) δ 3.77 (s, 3H), 3.37-3.29 (m, 3H), 3.11-2.97 (m, 2H), 2.38-2.25 (m, 2H), 1.72 (ddd, J=15.3, 11.7, 4.1 Hz, 2H), 1.30 (s, 3H).

Synthesis of methyl 1-(2-ethoxy-2-oxoethyl)-4-methylpiperidine-4-carboxylate: Into a 100-mL round-bottom flask, was placed methyl 4-methylpiperidine-4-carboxylate hydrochloride (250 g, 1.3 mol, 1.00 equiv), DMF (2500 mL), ethyl bromoacetate (229 g, 1.37 mmol, 1.05 equiv), K₂CO₃ (189.0 g, 1.37 mol, 1.05 equiv), TBAB (42 g, 0.13 mol, 0.10 equiv).

The resulting solution was stirred for 5 hr at room temperature. The reaction was then quenched by the addition of 1000 mL of water. The resulting solution was extracted with 2×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:1). This resulted in 220 g (70.0%) of methyl 1-(2-ethoxy-2-oxoethyl)-4-methylpiperidine-4-carboxylate as yellow oil. H-NMR (300 MHz, Chloroform-d, ppm) δ 4.18 (q, J=7.1 Hz, 2H), 3.70 (s, 3H), 3.17 (s, 2H), 2.84-2.69 (m, 2H), 2.31-2.09 (m, 4H), 1.58 (ddd, J=13.9, 10.5, 3.8 Hz, 2H), 1.27 (t, J=7.1 Hz, 3H), 1.20 (s, 3H).

Synthesis of ethyl 4-methyl-3-oxo-1-azabicyclo[2.2.2]octane-2-carboxylate: Into a 3000-mL round-bottom flask, was placed methyl 1-(2-ethoxy-2-oxoethyl)-4-methylpiperidine-4-carboxylate (200 g, 0.82 mol, 1.00 equiv), toluene (1000 mL), t-BuOK (188 g, 1.64 mol, 2.00 equiv). The resulting solution was stirred for 3 hr at 110 degrees C. in an oil bath. The reaction mixture was cooled to room temperature. The pH value of the solution was adjusted to 6 with HCl (2 mol/L). The resulting mixture was concentrated under vacuum. The residue was applied onto a silica gel column with dichloromethane/methanol (24:1). This resulted in 121 g (70.0%) of ethyl 4-methyl-3-oxo-1-azabicyclo[2.2.2]octane-2-carboxylate as yellow oil. LC-MS: (ES, m/z): M+1: 212

Synthesis of 4-methyl-1-azabicyclo[2.2.2]octan-3-one hydrochloride: Into a 100-mL round-bottom flask, was placed ethyl 4-methyl-3-oxo-1-azabicyclo[2.2.2]octane-2-carboxylate (120 g, 6.864 mmol, 1.00 equiv), conc. HCl (10.00 mL). The resulting solution was stirred overnight at 100 degrees C. in an oil bath. The reaction mixture was cooled to room temperature. The resulting mixture was concentrated under vacuum. The crude product was purified by re-crystallization from CH₃CN. The solids were collected by filtration. This resulted in 89 g (90.0%) of 4-methyl-1-azabicyclo[2.2.2]octan-3-one hydrochloride as a white solid. H-NMR (300 MHz, Methanol-d₄, ppm) δ 4.06 (t, J=1.3 Hz, 2H), 3.75-3.45 (m, 4H), 2.22 (ddd, J=13.4, 10.7, 5.5 Hz, 2H), 2.13-1.95 (m, 2H), 1.15 (s, 3H).

Synthesis of 2,2-bis(hydroxymethyl)-4-methyl-1-azabicyclo[2.2.2]octan-3-one: Into a 250-mL round-bottom flask, was placed 4-methyl-1-azabicyclo[2.2.2]octan-3-one hydrochloride (5.00 g, 28.464 mmol, 1.00 equiv), HCHO (46.33 g, 570.909 mmol, 20.06 equiv, 37%), K₂CO₃ (4.73 g, 34.224 mmol, 1.20 equiv). The resulting solution was stirred for 1 hr at 55 degrees C. in an oil bath. The reaction mixture was cooled to room temperature.

The resulting solution was diluted with 100 mL of water. The resulting solution was extracted with 3×100 mL of dichloromethane and the organic layers combined and dried over anhydrous sodium sulfate and concentrated under vacuum. The residue was applied onto a silica gel column with dichloromethane/methanol (5:1). The crude product was purified by re-crystallization from Et₂O. The solids were collected by filtration. This resulted in 870 mg (15.34%) of 2,2-bis(hydroxymethyl)-4-methyl-1-azabicyclo[2.2.2]octan-3-one as a white solid. LC-MS: (ES, m/z): M+1: 200; H-NMR (300 MHz, DMSO-d₆, ppm) δ 4.60 (t, J=5.8 Hz, 2H), 3.73 (dd, J=11.6, 5.5 Hz, 2H), 3.62 (dd, J=11.6, 6.0 Hz, 2H), 3.39-3.34 (m, 1H), 3.31-3.26 (m, 1H), 2.91-2.68 (m, 2H), 1.82-1.69 (m, 4H), 0.83 (s, 3H).

Synthesis of 2-(hydroxymethyl)-2-(isopropoxymethyl)-4-methyl-1-azabicyclo[2.2.2]octan-3-one: Into a 100-mL round-bottom flask, was placed 2,2-bis(hydroxymethyl)-4-methyl-1-azabicyclo[2.2.2]octan-3-one (300.00 mg, 1.506 mmol, 1.00 equiv), DCM (10.00 mL). This was followed by the addition of 2-iodopropane (333.00 mg, 1.959 mmol, 1.30 equiv) at −78 degrees C. To this was added CF₃SO₃Ag (854.00 mg, 3.336 mmol, 2.22 equiv) and 2,6-di-tert-butyl-4-methylpyridine (703.00 mg, 3.424 mmol, 2.27 equiv) at −78 degrees C. The resulting solution was stirred for 3 days at room temperature. The solids were filtered out. The resulting mixture was concentrated under vacuum. The residue was applied onto a silica gel column with dichloromethane/methanol (10:1). The crude product was purified by Prep-HPLC with the following conditions (2 #SHIMADZU (HPLC-01)): Column, XBridge Prep C18 OBD Column, Sum, 19*150 mm; mobile phase, Water (0.05% NH₃.H₂O) and ACN (14% Phase B up to 44% in 7 min); Detector, UV. This resulted in 40 mg (11.01%) of 2-(hydroxymethyl)-2-(isopropoxymethyl)-4-methyl-1-azabicyclo[2.2.2]octan-3-one as a brown solid. LC-MS: (ES, m/z): M+1: 242; H-NMR (300 MHz, Methanol-d₄, ppm) δ 3.95-3.69 (m, 4H), 3.67-3.38 (m, 3H), 3.04-2.82 (m, 2H), 2.00-1.74 (m, 4H), 1.14 (t, J=6.3 Hz, 6H), 0.94 (s, 3H).

### Example 9: Preparation of 4-fluoro-2-(hydroxymethyl)-2-(methoxymethyl)-1-azabicyclo[2.2.2]octan-3-one

Synthesis of 1-tert-butyl 4-methyl 4-fluoropiperidine-1,4-dicarboxylate: Into a 250-mL 3-necked round-bottom flask purged and maintained with an inert atmosphere of nitrogen, was placed 1-tert-butyl 4-methyl piperidine-1,4-dicarboxylate (8.00 g, 32.881 mmol, 1.00 equiv), THE (100.00 mL). This was followed by the addition of LDA (49.00 mL, 49.000 mmol, 1.49 equiv) dropwise with stirring at −78 degrees C. The resulting solution was stirred for 45 min at −78 degrees C. To this was added N-(benzenesulfonyl)-N-fluorobenzenesulfonamide (15.60 g, 49.472 mmol, 1.50 equiv), in portions at −78 degrees C.

The resulting solution was stirred for 6 hr at room temperature. The reaction was then quenched by the addition of 500 mL of aqueous NH₄Cl. The resulting solution was extracted with 3×200 mL of ethyl acetate and the organic layers combined. The resulting mixture was washed with 1×800 ml of brine. The mixture was dried over anhydrous sodium sulfate and concentrated under vacuum. The residue was applied onto a silica gel column with ethyl acetate/petroleum ether (1:4). This resulted in 8.1 g (94.28%) of 1-tert-butyl 4-methyl 4-fluoropiperidine-1,4-dicarboxylate as yellow oil. LC-MS: (ES, m/z): M-t-Bu+CH₃CN: 247 Synthesis of methyl 4-fluoropiperidine-4-carboxylate hydrochloride: Into a 250-mL round-bottom flask, was placed 1-tert-butyl 4-methyl 4-fluoropiperidine-1,4-dicarboxylate (8.10 g, 31.000 mmol, 1.00 equiv), HCl (gas) in 1,4-dioxane (4M, 80.00 mL). The resulting solution was stirred for 2 hr at room temperature. The resulting mixture was concentrated under vacuum. The crude product was re-crystallized from PE:EA in the ratio of 4:1. The solids were collected by filtration. This resulted in 5.09 g (83.08%) of methyl 4-fluoropiperidine-4-carboxylate hydrochloride as a white solid. H-NMR (300 MHz, Methanol-d4, ppm) δ 3.85 (s, 3H), 3.51-3.41 (m, 2H), 3.35-3.21 (m, 2H), 2.48-2.23 (m, 4H).

Synthesis of methyl 1-(2-ethoxy-2-oxoethyl)-4-fluoropiperidine-4-carboxylate: Into a 250-mL round-bottom flask, was placed methyl 4-fluoropiperidine-4-carboxylate hydrochloride (5.09 g, 25.755 mmol, 1.00 equiv), DMF (80.00 mL), ethyl bromoacetate (4.52 g, 27.043 mmol, 1.05 equiv), K₂CO₃ (3.74 g, 27.043 mmol, 1.05 equiv), TBAB (830.00 mg, 2.575 mmol, 0.10 equiv). The resulting solution was stirred overnight at room temperature. The reaction was then quenched by the addition of 300 mL of water. The resulting solution was extracted with 3×100 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:1). This resulted in 6 g (94.22%) of methyl 1-(2-ethoxy-2-oxoethyl)-4-fluoropiperidine-4-carboxylate as yellow oil. H-NMR (300 MHz, Chloroform-d, ppm) δ 4.20 (q, J=7.2 Hz, 2H), 3.79 (s, 3H), 3.24 (s, 2H), 2.92-2.79 (m, 2H), 2.52 (tdd, J=11.7, 3.0, 1.2 Hz, 2H), 2.37-2.09 (m, 2H), 1.97 (tdd, J=11.6, 4.1, 2.3 Hz, 2H), 1.28 (t, J=7.1 Hz, 3H).

Synthesis of ethyl 4-fluoro-3-oxo-1-azabicyclo[2.2.2]octane-2-carboxylate: Into a 100-mL round-bottom flask, was placed methyl 1-(2-ethoxy-2-oxoethyl)-4-fluoropiperidine-4-carboxylate (5.13 g, 20.747 mmol, 1.00 equiv), toluene (50.00 mL), t-BuOK (3.02 g, 26.913 mmol, 1.30 equiv). The resulting solution was stirred for 3 hr at 110 degrees C. in an oil bath. The reaction mixture was cooled to room temperature. The pH value of the solution was adjusted to 6 with HCl (6 mol/L). The resulting mixture was concentrated under vacuum. The residue was applied onto a silica gel column with dichloromethane/methanol (10:1). This resulted in 3 g (67.19%) of ethyl 4-fluoro-3-oxo-1-azabicyclo[2.2.2]octane-2-carboxylate as a yellow solid. LC-MS: (ES, m/z): M+1: 216.

Synthesis of 4-fluoro-1-azabicyclo[2.2.2]octan-3-one: Into a 100-mL round-bottom flask, was placed ethyl 4-fluoro-3-oxo-1-azabicyclo[2.2.2]octane-2-carboxylate (3.00 g, 13.939 mmol, 1.00 equiv), HCl (6N, 30.00 mL). The resulting solution was stirred overnight at 100 degrees C. in an oil bath. The reaction mixture was cooled to room temperature. The pH value of the solution was adjusted to 7 with NaOH (2 mol/L). The resulting mixture was concentrated under vacuum. The residue was applied onto a silica gel column with dichloromethane/methanol (10:1). This resulted in 1.5 g (75.17%) of 4-fluoro-1-azabicyclo[2.2.2]octan-3-one as a white solid. H-NMR (300 MHz, DMSO-d₆, ppm) δ 3.38 (d, J=2.4 Hz, 2H), 3.09 (td, J=7.7, 2.1 Hz, 4H), 2.25-1.96 (m, 4H).

Synthesis of 4-fluoro-2-(hydroxymethyl)-2-(methoxymethyl)-1-azabicyclo[2.2.2]octan-3-one: Into a 100-mL round-bottom flask, was placed 4-fluoro-1-azabicyclo[2.2.2]octan-3-one (300.00 mg, 2.096 mmol, 1.00 equiv), MeOH (9.00 mL), H₂O (3.00 mL), K₂CO₃ (232.00 mg, 1.679 mmol, 0.80 equiv), HCHO (486.00 mg, 5.989 mmol, 2.86 equiv, 37%). The resulting solution was stirred for 3 hr at 60 degrees C. in an oil bath. The reaction mixture was cooled to room temperature. The resulting mixture was concentrated under vacuum. The residue was applied onto a silica gel column with dichloromethane/methanol (20:1). The crude product was purified by Prep-HPLC with the following conditions (2 #SHIMADZU (HPLC-01)): Column, XBridge Prep C18 OBD Column, 5 um, 19*150 mm; mobile phase, Water (0.05% NH₃.H₂O) and ACN (2% Phase B up to 27% in 7 min); Detector, UV. This resulted in 2.9 mg (0.64%) of 4-fluoro-2-(hydroxymethyl)-2-(methoxymethyl)-1-azabicyclo[2.2.2]octan-3-one as a white solid. LC-MS: (ES, m/z): M+1: 218; H-NMR (300 MHz, Methanol-d₄, ppm) δ 3.98-3.53 (m, 5H), 3.41-3.35 (m, 1H), 3.34 (s, 3H), 3.11 (dt, J=14.6, 8.2 Hz, 2H), 2.21 (ddt, J=8.5, 6.3, 3.9 Hz, 4H).

### Example 10: Preparation of 2-(hydroxymethyl)-2-(methoxymethyl)-4-(trifluoromethyl)-1-azabicyclo[2.2.2]octan-3-one

Synthesis of 1-tert-butyl 4-methyl 4-(trifluoromethyl)piperidine-1,4-dicarboxylate: Into a 500-mL 3-necked round-bottom flask purged and maintained with an inert atmosphere of nitrogen, was placed 1-tert-butyl 4-methyl piperidine-1,4-dicarboxylate (4.5 g, 1.00 equiv), THE (200 mL). This was followed by the addition of LDA (20 mL, 2 equiv) dropwise with stirring at −78 degrees C. The resulting solution was stirred for 45 min at −78 degrees C. To this was added 8-(trifluoromethyl)-8-thiatricyclo[7.4.0.0\hat{ }[2,7]]trideca-1(9),2,4,6,10,12-hexaen-8-ium triflate (14.8 g, 2 equiv), in portions at −78 degrees C. The resulting solution was stirred overnight at room temperature. The reaction was then quenched by the addition of 30 mL of water. The resulting solution was extracted with 3×100 mL of ethyl acetate and the organic layers combined. The resulting mixture was washed with 1×500 mL of brine. The mixture was dried over anhydrous sodium sulfate and concentrated under vacuum. The residue was applied onto a silica gel column with ethyl acetate/petroleum ether (1:1). This resulted in 1.4 g of 1-tert-butyl 4-methyl 4-(trifluoromethyl)piperidine-1,4-dicarboxylate as a white solid. H-NMR (300 MHz, DMSO-d₆, ppm) δ 3.97 (d, J=16.2 Hz, 2H), 3.80 (s, 3H), 2.80 (d, J=16.2 Hz, 2H), 2.20 (dd, J=13.2, 2.4 Hz, 2H).

Synthesis of methyl 4-(trifluoromethyl)piperidine-4-carboxylate hydrochloride: Into a 100-mL round-bottom flask, was placed 1-tert-butyl 4-methyl 4-(trifluoromethyl)piperidine-1,4-dicarboxylate (1.60 g, 1 equiv), HCl (gas) in 1,4-dioxane (20 mL, 2M). The resulting solution was stirred for 2 hr at room temperature. The resulting mixture was concentrated. This resulted in 700 mg (64.49%) of methyl 4-(trifluoromethyl)piperidine-4-carboxylate hydrochloride as a white solid. LC-MS: (ES, m/z): M+H: 212.

Synthesis of methyl 1-(2-ethoxy-2-oxoethyl)-4-(trifluoromethyl)piperidine-4-carboxylate: Into a 50-mL round-bottom flask, was placed methyl 4-(trifluoromethyl)piperidine-4-carboxylate hydrochloride (750.00 mg, 3.551 mmol, 1.00 equiv), CH₃CN (15.00 mL, 285.370 mmol, 80.35 equiv), K₂CO₃ (1472.47 mg, 10.654 mmol, 3.00 equiv), ethyl bromoacetate (889.64 mg, 5.327 mmol, 1.50 equiv). The resulting solution was stirred overnight at room temperature. The resulting mixture was concentrated. The residue was applied onto a silica gel column with ethyl acetate/petroleum ether (1:3). This resulted in 700 mg (66.30%) of methyl 1-(2-ethoxy-2-oxoethyl)-4-(trifluoromethyl)piperidine-4-carboxylate as a white solid. H-NMR (300 MHz, DMSO-d₆, ppm) δ 4.07 (q, J=7.2 Hz, 2H), 3.78 (s, 3H), 2.86 (d, J=11.7 Hz, 2H), 2.25-2.11 (m, 4H), 1.76 (td, J=13.2, 4.2 Hz, 2H), 1.18 (t, J=7.2 Hz, 4H).

Synthesis of ethyl 3-oxo-4-(trifluoromethyl)-1-azabicyclo[2.2.2]octane-2-carboxylate: Into a 100-mL round-bottom flask, was placed methyl 1-(2-ethoxy-2-oxoethyl)-4-(trifluoromethyl)piperidine-4-carboxylate (900.00 mg, 3.028 mmol, 1.00 equiv), toluene (10.00 mL), t-BuOK (509.00 mg, 4.536 mmol, 1.50 equiv). The resulting solution was stirred for 3 hr at 110 degrees C. in an oil bath. The reaction mixture was cooled to room temperature. The pH value of the solution was adjusted to 6 with HCl (6 mol/L). The resulting mixture was concentrated under vacuum. The residue was applied onto a silica gel column with CH₂Cl₂. This resulted in 410 mg (51.06%) of ethyl 3-oxo-4-(trifluoromethyl)-1-azabicyclo[2.2.2]octane-2-carboxylate as a yellow solid. LC-MS: (ES, m/z): M+H: 266.

Synthesis of 4-(trifluoromethyl)-1-azabicyclo[2.2.2]octan-3-one: Into a 100-mL round-bottom flask, was placed ethyl 3-oxo-4-(trifluoromethyl)-1-azabicyclo[2.2.2]octane-2-carboxylate (410.00 mg, 1.546 mmol, 1.00 equiv), HCl (6N, 5.00 mL). The resulting solution was stirred overnight at 100 degrees C. in an oil bath. The reaction mixture was cooled to room temperature. The pH value of the solution was adjusted to 6 with NaOH (4 mol/L). The resulting mixture was concentrated under vacuum. The residue was applied onto a silica gel column with dichloromethane/methanol (10:1). This resulted in 116 mg (38.85%) of 4-(trifluoromethyl)-1-azabicyclo[2.2.2]octan-3-one as a yellow solid. H-NMR (300 MHz, DMSO-d₆, ppm) δ 3.29 (s, 2H), 2.97 (t, J=7.7 Hz, 4H), 2.05 (dqt, J=14.0, 9.0, 4.3 Hz, 4H).

Synthesis of 2-(hydroxymethyl)-2-(methoxymethyl)-4-(trifluoromethyl)-1-azabicyclo[2.2.2]octan-3-one: Into a 50-mL round-bottom flask, was placed 4-(trifluoromethyl)-1-azabicyclo[2.2.2]octan-3-one (110.00 mg, 0.569 mmol, 1.00 equiv), MeOH (6.00 mL), H₂O (2.00 mL), K₂CO₃ (236.00 mg, 1.708 mmol, 3.00 equiv), HCHO (462.00 mg, 5.693 mmol, 10.00 equiv, 37%). The resulting solution was stirred for 6 hr at 70 degrees C. in an oil bath. The reaction mixture was cooled to room temperature. The resulting mixture was concentrated under vacuum. The residue was applied onto a silica gel column with dichloromethane/methanol (20:1). The crude product was purified by Prep-HPLC with the following conditions (2 #SHIMADZU (HPLC-01)): Column, XBridge Prep C18 OBD Column, 5 um, 19*150 mm; mobile phase, Water (0.05% NH₃.H₂O) and ACN (11% Phase B up to 47% in 7 min); Detector, UV. This resulted in 1.5 mg (0.99%) of 2-(hydroxymethyl)-2-(methoxymethyl)-4-(trifluoromethyl)-1-azabicyclo[2.2.2]octan-3-one as a white solid. LC-MS: (ES, m/z): M+H: 268; H-NMR (300 MHz, Methanol-d₄, ppm) δ 3.96-3.65 (m, 4H), 3.63-3.45 (m, 2H), 3.33 (s, 3H), 3.00 (dt, J=15.1, 7.9 Hz, 2H), 2.31-2.12 (m, 4H).

### Example 11: Preparation of (2S)-2-(tert-butoxymethyl)-2-(hydroxymethyl)-4-methyl-1-azabicyclo[2.2.2]octan-3-one (Assumed) and (2R)-2-(tert-butoxymethyl)-2-(hydroxymethyl)-4-methyl-1-azabicyclo[2.2.2]octan-3-one (Assumed)

Synthesis of 1-tert-butyl 4-methyl 4-methylpiperidine-1,4-dicarboxylate: Into a 10000-mL 3-necked round-bottom flask purged and maintained with an inert atmosphere of nitrogen, was placed 1-tert-butyl 4-methyl piperidine-1,4-dicarboxylate (400.0 g, 1.65 mol, 1.00 equiv), THE (4000.00 mL). This was followed by the addition of LDA (2150 mL, 2.15 mol, 1.30 equiv) dropwise with stirring at −78 degrees C. The mixture was stirred at −78 degrees C. for 45 min. To this was added CH₃I (369.0 g, 2.6 mol, 1.60 equiv) dropwise with stirring at −78 degrees C. The resulting solution was stirred for 5 hr at room temperature. The reaction was then quenched by the addition of 3000 mL of aqueous NH₄Cl. The resulting solution was extracted with 3×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:9). This resulted in 400 g (94.5%) of 1-tert-butyl 4-methyl 4-methylpiperidine-1,4-dicarboxylate as yellow oil. ¹H NMR (300 MHz, Chloroform-d, ppm) δ 3.81-3.73 (m, 2H), 3.72 (s, 3H), 3.00 (ddd, J=13.8, 10.6, 3.1 Hz, 2H), 2.13-2.03 (m, 2H), 1.46 (s, 9H), 1.38 (ddd, J=14.2, 10.6, 4.2 Hz, 2H), 1.22 (s, 3H).

Synthesis of methyl 4-methylpiperidine-4-carboxylate hydrochloride: Into a 5000-mL round-bottom flask, was placed 1-tert-butyl 4-methyl 4-methylpiperidine-1,4-dicarboxylate (380 g, 1.48 mol, 1.00 equiv), HCl (gas) in 1,4-dioxane (4 M, 3000 mL). The resulting solution was stirred for 6 h at room temperature. The resulting mixture was concentrated under vacuum. This resulted in 251 g (88%) of methyl 4-methylpiperidine-4-carboxylate hydrochloride as a white solid. ¹H NMR (300 MHz, Methanol-d4, ppm) δ 3.77 (s, 3H), 3.37-3.29 (m, 3H), 3.11-2.97 (m, 2H), 2.38-2.25 (m, 2H), 1.72 (ddd, J=15.3, 11.7, 4.1 Hz, 2H), 1.30 (s, 3H).

Synthesis of methyl 1-(2-ethoxy-2-oxoethyl)-4-methylpiperidine-4-carboxylate: Into a 5 L round-bottom flask, was placed methyl 4-methylpiperidine-4-carboxylate hydrochloride (250 g, 1.3 mol, 1.00 equiv), DMF (2500 mL), ethyl bromoacetate (229 g, 1.37 mmol, 1.05 equiv), K₂CO₃ (189.0 g, 1.37 mol, 1.05 equiv), TBAB (42 g, 0.13 mol, 0.10 equiv). The resulting solution was stirred for 5 hr at room temperature. The reaction was then quenched by the addition of 1000 mL of water. The resulting solution was extracted with 2×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:1). This resulted in 220 g (70.0%) of methyl 1-(2-ethoxy-2-oxoethyl)-4-methylpiperidine-4-carboxylate as yellow oil. ¹H NMR (300 MHz, Chloroform-d, ppm) δ 4.18 (q, J=7.1 Hz, 2H), 3.70 (s, 3H), 3.17 (s, 2H), 2.84-2.69 (m, 2H), 2.31-2.09 (m, 4H), 1.58 (ddd, J=13.9, 10.5, 3.8 Hz, 2H), 1.27 (t, J=7.1 Hz, 3H), 1.20 (s, 3H).

Synthesis of ethyl 4-methyl-3-oxo-1-azabicyclo[2.2.2]octane-2-carboxylate: Into a 3000-mL round-bottom flask, was placed methyl 1-(2-ethoxy-2-oxoethyl)-4-methylpiperidine-4-carboxylate (200 g, 0.82 mol, 1.00 equiv), toluene (1000 mL), t-BuOK (188 g, 1.64 mol, 2.00 equiv). The resulting solution was stirred for 3 hr at 110 degrees C. in an oil bath. The reaction mixture was cooled to room temperature. The pH value of the solution was adjusted to 6 with HCl (2 mol/L). The resulting mixture was concentrated under vacuum. The residue was applied onto a silica gel column with dichloromethane/methanol (24:1). This resulted in 121 g (70.0%) of ethyl 4-methyl-3-oxo-1-azabicyclo[2.2.2]octane-2-carboxylate as yellow oil. LC-MS: (ES, m/z): M+1: 212 Synthesis of 4-methyl-1-azabicyclo[2.2.2]octan-3-one hydrochloride: Into a 250-mL round-bottom flask, was placed ethyl 4-methyl-3-oxo-1-azabicyclo[2.2.2]octane-2-carboxylate (120 g, 6.864 mmol, 1.00 equiv), conc. HCl (10.00 mL). The resulting solution was stirred overnight at 100 degrees C. in an oil bath. The reaction mixture was cooled to room temperature. The resulting mixture was concentrated under vacuum. The crude product was purified by re-crystallization from CH₃CN. The solids were collected by filtration. This resulted in 89 g (90.0%) of 4-methyl-1-azabicyclo[2.2.2]octan-3-one hydrochloride as a white solid. ¹H NMR (300 MHz, Methanol-d4, ppm) δ 4.06 (t, J=1.3 Hz, 2H), 3.75-3.45 (m, 4H), 2.22 (ddd, J=13.4, 10.7, 5.5 Hz, 2H), 2.13-1.95 (m, 2H), 1.15 (s, 3H).

Synthesis of 2,2-bis(hydroxymethyl)-4-methyl-1-azabicyclo[2.2.2]octan-3-one: Into a 250-mL round-bottom flask, was placed 4-methyl-1-azabicyclo[2.2.2]octan-3-one hydrochloride (5.00 g, 28.464 mmol, 1.00 equiv), HCHO (46.33 g, 570.909 mmol, 20.06 equiv, 37%), K₂CO₃ (4.73 g, 34.224 mmol, 1.20 equiv). The resulting solution was stirred for 1 h at 55 degrees C. in an oil bath. The reaction mixture was cooled to room temperature. The resulting solution was diluted with 100 mL of water. The resulting solution was extracted with 3×100 mL of dichloromethane and the organic layers combined and dried over anhydrous sodium sulfate and concentrated under vacuum. The residue was applied onto a silica gel column with dichloromethane/methanol (5:1). The crude product was purified by re-crystallization from Et₂O. The solids were collected by filtration. This resulted in 870 mg (15.34%) of 2,2-bis(hydroxymethyl)-4-methyl-1-azabicyclo[2.2.2]octan-3-one as a white solid. LC-MS: (ES, m/z): M+1: 200; H-NMR (300 MHz, DMSO-d₆, ppm) δ 4.60 (t, J=5.8 Hz, 2H), 3.73 (dd, J=11.6, 5.5 Hz, 2H), 3.62 (dd, J=11.6, 6.0 Hz, 2H), 3.39-3.34 (m, 1H), 3.31-3.26 (m, 1H), 2.91-2.68 (m, 2H), 1.82-1.69 (m, 4H), 0.83 (s, 3H).

Synthesis of (2S)-2-(tert-butoxymethyl)-2-(hydroxymethyl)-4-methyl-1-azabicyclo[2.2.2]octan-3-one (Assumed): Into a 50-mL 3-necked round-bottom flask, was placed 2,2-bis(hydroxymethyl)-4-methyl-1-azabicyclo[2.2.2]octan-3-one (300.00 mg, 1.506 mmol, 1.00 equiv), DCM (20.00 mL). This was followed by the addition of propane, 2-iodo-2-methyl- (332.48 mg, 1.807 mmol, 1.20 equiv) at −78 degrees C. To this was added AgO₃SCF₃ (851.83 mg, 3.327 mmol, 2.21 equiv) at −78 degrees. To the mixture was added 4-Me-2,6-(t-Bu)₂-Py (700.65 mg, 3.418 mmol, 2.27 equiv) at −78 degrees C. The resulting solution was stirred for 3 days at room temperature. The solids were filtered out. The resulting mixture was concentrated. The crude product was purified by Prep-HPLC with the following conditions (XBridge Prep C18 OBD): Column, Sum, 19*150 mm; mobile phase, A: Water (0.05% NH₃H₂O), Mobile Phase B: ACN; Flow rate: 20 mL/min; Gradient: 18 B to 48 B in 7 min; Detector, 220 nm. The product was purified by Chiral-Prep-HPLC with the following conditions (XA-Prep Chiral HPLC-02): Column, CHIRALPAK IG, 3*25 cm, Sum, mobile phase, B: EtOH-HPLC; Flow rate: 35 mL/min; Gradient: 25 B to 25 B in 13 min; Detector, 220 nm. This resulted in 3.2 mg (0.83%) of (2S)-2-(tert-butoxymethyl)-2-(hydroxymethyl)-4-methyl-1-azabicyclo[2.2.2]octan-3-one (Assumed) as a white solid. LC-MS: (ES, m/z): M+1: 256, ¹H NMR (300 MHz, CDCL₃, ppm) δ 4.04-3.70 (m, 4H), 3.53-3.24 (m, 3H), 3.11-2.93 (m, 2H), 1.99-1.81 (m, 3H), 1.25 (s, 9H), 0.98 (s, 3H).

Synthesis of (2R)-2-(tert-butoxymethyl)-2-(hydroxymethyl)-4-methyl-1-azabicyclo[2.2.2]octan-3-one (Assumed): Into a 50-mL 3-necked round-bottom flask, was placed 2,2-bis(hydroxymethyl)-4-methyl-1-azabicyclo[2.2.2]octan-3-one (300.00 mg, 1.506 mmol, 1.00 equiv), DCM. This was followed by the addition of propane, 2-iodo-2-methyl-(332.48 mg, 1.807 mmol, 1.20 equiv) at −78 degrees C. To this was added AgO₃SCF₃ (851.83 mg, 3.327 mmol, 2.21 equiv) at −78 degrees C. To the mixture was added 4-Me-2,6-(t-Bu)₂-Py (700.65 mg, 3.418 mmol, 2.27 equiv) at −78 degrees C. The resulting solution was stirred for 3 days at room temperature. The solids were filtered out. The resulting mixture was concentrated. The crude product was purified by Prep-HPLC with the following conditions (XBridge Prep C18 OBD): Column, Sum, 19*150 mm; mobile phase, A: Water (0.05% NH₃H₂O), Mobile Phase B: ACN; Flow rate: 20 mL/min; Gradient: 18 B to 48 B in 7 min; Detector, 220 nm. The product was purified by Chiral-Prep-HPLC with the following conditions (CHIRALPAK IG): Column, 3*25 cm, 5 um; mobile phase, A: Hex-HPLC, Mobile Phase B: EtOH-HPLC; Flow rate: 35 mL/min; Gradient: 25 B to 25 B in 13 min; 220 nm; Detector, 220 nm. This resulted in 3.7 mg (0.96%) of (2R)-2-(tert-butoxymethyl)-2-(hydroxymethyl)-4-methyl-1-azabicyclo[2.2.2]octan-3-one (Assumed) as a white solid. LC-MS: (ES, m/z): M+1: 256, ¹H NMR (300 MHz, CDCL₃, ppm) δ 4.04-3.70 (m, 4H), 3.53-3.24 (m, 3H), 3.11-2.93 (m, 2H), 1.99-1.81 (m, 3H), 1.25 (s, 9H), 0.98 (s, 3H).

### Example 12: Preparation of 2-(hydroxymethyl)-4-methyl-2-[(2-methylpropoxy)methyl]-1-azabicyclo[2.2.2]octan-3-one

Synthesis of 1-tert-butyl 4-methyl 4-methylpiperidine-1,4-dicarboxylate: Into a 10000-mL 3-necked round-bottom flask purged and maintained with an inert atmosphere of nitrogen, was placed 1-tert-butyl 4-methyl piperidine-1,4-dicarboxylate (400.0 g, 1.65 mol, 1.00 equiv), THE (4000.00 mL). This was followed by the addition of LDA (2150 mL, 2.15 mol, 1.30 equiv) dropwise with stirring at −78 degrees C. The mixture was stirred at −78 degrees C. for 45 min. To this was added CH₃I (369.0 g, 2.6 mol, 1.60 equiv) dropwise with stirring at −78 degrees C. The resulting solution was stirred for 5 hr at room temperature. The reaction was then quenched by the addition of 3000 mL of aqueous NH₄Cl. The resulting solution was extracted with 3×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:9). This resulted in 400 g (94.5%) of 1-tert-butyl 4-methyl 4-methylpiperidine-1,4-dicarboxylate as yellow oil. ¹H NMR (300 MHz, Chloroform-d, ppm) δ 3.81-3.73 (m, 2H), 3.72 (s, 3H), 3.00 (ddd, J=13.8, 10.6, 3.1 Hz, 2H), 2.13-2.03 (m, 2H), 1.46 (s, 9H), 1.38 (ddd, J=14.2, 10.6, 4.2 Hz, 2H), 1.22 (s, 3H).

Synthesis of methyl 4-methylpiperidine-4-carboxylate hydrochloride: Into a 5000-mL round-bottom flask, was placed 1-tert-butyl 4-methyl 4-methylpiperidine-1,4-dicarboxylate (380 g, 1.48 mol, 1.00 equiv), HCl (gas) in 1,4-dioxane (4 M, 3000 mL). The resulting solution was stirred for 6 h at room temperature. The resulting mixture was concentrated under vacuum. This resulted in 251 g (88%) of methyl 4-methylpiperidine-4-carboxylate hydrochloride as a white solid. ¹H NMR (300 MHz, Methanol-d4, ppm) δ 3.77 (s, 3H), 3.37-3.29 (m, 3H), 3.11-2.97 (m, 2H), 2.38-2.25 (m, 2H), 1.72 (ddd, J=15.3, 11.7, 4.1 Hz, 2H), 1.30 (s, 3H).

Synthesis of methyl 1-(2-ethoxy-2-oxoethyl)-4-methylpiperidine-4-carboxylate: Into a 5 L round-bottom flask, was placed methyl 4-methylpiperidine-4-carboxylate hydrochloride (250 g, 1.3 mol, 1.00 equiv), DMF (2500 mL), ethyl bromoacetate (229 g, 1.37 mmol, 1.05 equiv), K₂CO₃ (189.0 g, 1.37 mol, 1.05 equiv), TBAB (42 g, 0.13 mol, 0.10 equiv). The resulting solution was stirred for 5 hr at room temperature. The reaction was then quenched by the addition of 1000 mL of water. The resulting solution was extracted with 2×2000 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:1). This resulted in 220 g (70.0%) of methyl 1-(2-ethoxy-2-oxoethyl)-4-methylpiperidine-4-carboxylate as yellow oil. ¹H NMR (300 MHz, Chloroform-d, ppm) δ 4.18 (q, J=7.1 Hz, 2H), 3.70 (s, 3H), 3.17 (s, 2H), 2.84-2.69 (m, 2H), 2.31-2.09 (m, 4H), 1.58 (ddd, J=13.9, 10.5, 3.8 Hz, 2H), 1.27 (t, J=7.1 Hz, 3H), 1.20 (s, 3H).

Synthesis of ethyl 4-methyl-3-oxo-1-azabicyclo[2.2.2]octane-2-carboxylate: Into a 3000-mL round-bottom flask, was placed methyl 1-(2-ethoxy-2-oxoethyl)-4-methylpiperidine-4-carboxylate (200 g, 0.82 mol, 1.00 equiv), toluene (1000 mL), t-BuOK (188 g, 1.64 mol, 2.00 equiv). The resulting solution was stirred for 3 hr at 110 degrees C. in an oil bath. The reaction mixture was cooled to room temperature. The pH value of the solution was adjusted to 6 with HCl (2 mol/L). The resulting mixture was concentrated under vacuum. The residue was applied onto a silica gel column with dichloromethane/methanol (24:1). This resulted in 121 g (70.0%) of ethyl 4-methyl-3-oxo-1-azabicyclo[2.2.2]octane-2-carboxylate as yellow oil. LC-MS: (ES, m/z): M+1: 212 Synthesis of 4-methyl-1-azabicyclo[2.2.2]octan-3-one hydrochloride: Into a 100-mL round-bottom flask, was placed ethyl 4-methyl-3-oxo-1-azabicyclo[2.2.2]octane-2-carboxylate (120 g, 6.864 mmol, 1.00 equiv), conc. HCl (10.00 mL). The resulting solution was stirred overnight at 100 degrees C. in an oil bath. The reaction mixture was cooled to room temperature. The resulting mixture was concentrated under vacuum. The crude product was purified by re-crystallization from CH₃CN. The solids were collected by filtration. This resulted in 89 g (90.0%) of 4-methyl-1-azabicyclo[2.2.2]octan-3-one hydrochloride as a white solid. ¹H NMR (300 MHz, Methanol-d₄, ppm) δ 4.06 (t, J=1.3 Hz, 2H), 3.75-3.45 (m, 4H), 2.22 (ddd, J=13.4, 10.7, 5.5 Hz, 2H), 2.13-1.95 (m, 2H), 1.15 (s, 3H).

Synthesis of 2,2-bis(hydroxymethyl)-4-methyl-1-azabicyclo[2.2.2]octan-3-one: Into a 250-mL round-bottom flask, was placed 4-methyl-1-azabicyclo[2.2.2]octan-3-one hydrochloride (5.00 g, 28.464 mmol, 1.00 equiv), HCHO (46.33 g, 570.909 mmol, 20.06 equiv, 37%), K₂CO₃ (4.73 g, 34.224 mmol, 1.20 equiv). The resulting solution was stirred for 1 h at 55 degrees C. in an oil bath. The reaction mixture was cooled to room temperature. The resulting solution was diluted with 100 mL of water. The resulting solution was extracted with 3×100 mL of dichloromethane and the organic layers combined and dried over anhydrous sodium sulfate and concentrated under vacuum. The residue was applied onto a silica gel column with dichloromethane/methanol (5:1). The crude product was purified by re-crystallization from Et₂O. The solids were collected by filtration. This resulted in 870 mg (15.34%) of 2,2-bis(hydroxymethyl)-4-methyl-1-azabicyclo[2.2.2]octan-3-one as a white solid. LC-MS: (ES, m/z): M+1: 200; ¹H-NMR (300 MHz, DMSO-d₆, ppm) δ 4.60 (t, J=5.8 Hz, 2H), 3.73 (dd, J=11.6, 5.5 Hz, 2H), 3.62 (dd, J=11.6, 6.0 Hz, 2H), 3.39-3.34 (m, 1H), 3.31-3.26 (m, 1H), 2.91-2.68 (m, 2H), 1.82-1.69 (m, 4H), 0.83 (s, 3H).

Synthesis of 2-(hydroxymethyl)-4-methyl-2-[(2-methylpropoxy)methyl]-1-azabicyclo[2.2.2]octan-3-one: Into a 100-mL 3-necked round-bottom flask, was placed 2,2-bis(hydroxymethyl)-4-methyl-1-azabicyclo[2.2.2]octan-3-one (600.00 mg, 3.011 mmol, 1.00 equiv), DCM (40.00 mL). This was followed by the addition of 1-iodo-2-methylpropane (664.97 mg, 3.614 mmol, 1.20 equiv) at −78 degrees C. To this was added AgO₃SCF₃ (1703.67 mg, 6.655 mmol, 2.21 equiv) at −78 degrees C. To the mixture was added 4-Me-2,6-(t-Bu)₂-Py (1401.30 mg, 6.836 mmol, 2.27 equiv) at −78 degrees C. The resulting solution was stirred for 3 days at room temperature. The solids were filtered out. The resulting mixture was concentrated. The crude product was purified by Prep-HPLC with the following conditions (2 #SHIMADZU (HPLC-01)): Column, XBridge Prep C18 OBD Column, Sum, 19*150 mm; mobile phase, Water (0.05%/NH₃H₂O) and ACN (18% PhaseB up to 49% in 7 min); Detector, 220 nm. This resulted in 15 mg (1.95%) of 2-(hydroxymethyl)-4-methyl-2-[(2-methylpropoxy)methyl]-1-azabicyclo[2.2.2]octan-3-one as a brown solid. LC-MS: (ES, m/z): M+1: 256, ¹H NMR (300 MHz, Chloroform-d) δ 4.04-3.70 (m, 4H), 3.53-3.24 (m, 3H), 3.11-2.93 (m, 2H), 1.99-1.81 (m, 4H), 1.74-1.61 (m, 2H), 1.25 (s, 6H), 0.98 (s, 3H).

### Example 12: Preparation of bis(((S)-2-(methoxymethyl)-3-oxoquinuclidin-2-yl)methyl) carbonate (Assumed)

Synthesis of 2-(hydroxymethyl)-2-(methoxymethyl)-1-azabicyclo [2.2.2] octan-3-one: A 1000-mL round-bottom flask was placed quinuclidin-3-one (50.00 g, 399.45 mmol). This was followed by the addition of K₂CO₃ (55.21 g, 399.45 mmol, 1.00 equiv), H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of (2S)-2-(hydroxymethyl)-2-(methoxymethyl)-1-azabicyclo[2.2.2]octan-3-one (Assumed): The 450 mg of 2-(hydroxymethyl)-2-(methoxymethyl)-1-azabicyclo[2.2.2]octan-3-one was purified by Chiral-Prep-HPLC with the following conditions: Column, Lux Cellulose-4,100*4.6 mm, 3 um H19-381245; mobile phase A: n-Hexane (0.1% DEA); mobile phase B: Ethanol; Flow rate: 1.0 ml/min; Gradient: 0% B to 15% B in 7 min; Detector, 220 nm. This resulted in 190 mg of (2S)-2-(hydroxymethyl)-2-(methoxymethyl)-1-azabicyclo [2.2.2]octan-3-one (Assumed) as an off-white solid. LC-MS: (ES, m/z): M+1: 200; ee=100%. ¹H NMR (300 MHz, Methanol-d₄) δ 3.86 (d, J=4.8 Hz, 2H), 3.83-3.64 (m, 2H), 3.44-3.47 (m, 2H), 3.33-3.32 (m, 3H), 2.97-2.81 (m, 2H), 2.36-2.32 (m, 1H), 2.10-2.02 (m, 4H).

Synthesis of bis(((S)-2-(methoxymethyl)-3-oxoquinuclidin-2-yl)methyl) carbonate (Assumed): A 50 ml round bottom flask was placed (2S)-2-(hydroxymethyl)-2-(methoxymethyl)-1-azabicyclo [2.2.2] octan-3-one (Assumed) (70 mg, 0.35 mmol), CDI (50 mg, 035 mmol) and EA (5 ml). The resulting solution was stirred for 48 h at 60° C. The resulting mixture was concentrated under reduced pressure. The crude product was purified by Prep-HPLC with the following conditions (Column: Kinetex EVO C18 Column, 21.2*150, Sum; Mobile Phase A: Water (0.05% NH₃H₂O), Mobile Phase B: ACN; Flow rate: 20 mL/min; Gradient: 13% B to 26% B in 8 min, 26% B; Wave Length: 220 NM nm; RT1 (min): 7.6;) to bis(((S)-2-(methoxymethyl)-3-oxoquinuclidin-2-yl)methyl) carbonate (16 mg) (Assumed) as a white solid. LC-MS: (ES, m/z): M+1: 425 HNMR: ¹H NMR (300 MHz, Chloroform-d) δ 4.60-4.45 (m, 2H), 3.99 (d, J=11.7 Hz, 1H), 3.82 (s, 3H), 3.77-3.64 (m, 2H), 3.65-3.25 (m, 10H), 3.01-2.85 (m, 4H), 2.43 (p, J=3.0 Hz, 2H), 2.17-1.99 (m, 8H).

### Example 13: Preparation of bis((2-(methoxymethyl)-3-oxoquinuclidin-2-yl)methyl) piperazine-1,4-dicarboxylate

Synthesis of 2-(hydroxymethyl)-2-(methoxymethyl)-1-azabicyclo[2.2.2]octan-3-one: A 1000-mL round-bottom flask was placed quinuclidin-3-one (50.00 g, 399.45 mmol). This was followed by the addition of K₂CO₃ (55.21 g, 399.45 mmol, 1.00 equiv), H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. The mixture was dried over anhydrous sodium sulfate and concentrated. The residue was applied onto a silica gel column with ethyl acetate/petroleum ether (0:1-1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of bis((2-(methoxymethyl)-3-oxoquinuclidin-2-yl)methyl) piperazine-1,4-dicarboxylate A 50 ml round bottom flask was placed 2-(hydroxymethyl)-2-(methoxymethyl)-1-azabicyclo[2.2.2]octan-3-one (300 mg, 1.5 mmol), CDI (317 mg, 1.95 mmol) and DCE (10 ml). The resulting solution was stirred for 4 h at 50° C. Piperazine (77 mg, 0.9 mmol) was added and the mixture was stirred for 24 h at 50° C. The resulting mixture was concentrated under reduced pressure. The crude product was purified by Prep-HPLC with the following conditions (Column: Kinetex EVO C18 Column, 21.2*150, Sum; Mobile Phase A: Water (0.05% NH₃H₂O), Mobile Phase B: ACN; Flow rate: 20 mL/min; Gradient: 13% B to 26% B in 8 min, 26% B; Wave Length: 220 NM nm; RT1 (min): 7.6;) to afford bis((2-(methoxymethyl)-3-oxoquinuclidin-2-yl)methyl) piperazine-1,4-dicarboxylate (20 mg) as a white solid. LC-MS (ES, m/z): M+1: 537. ¹H NMR (300 MHz, Chloroform-d) δ 4.57 (d, J=11.7 Hz, 2H), 4.44 (d, J=11.7 Hz, 2H), 3.70 (s, 4H), 3.51 (s, 8H), 3.42-3.23 (m, 10H), 2.94 (dt, J=15.2, 8.3 Hz, 4H), 2.47-2.38 (m, 2H), 2.04 (td, J=7.8, 6.3, 3.3 Hz, 8H).

### Example 13: Preparation of bis((2-(methoxymethyl)-3-oxoquinuclidin-2-yl)methyl)((1R,3S)-cyclohexane-1,3-diyl)dicarbamate

Synthesis of 2-(hydroxymethyl)-2-(methoxymethyl)-1-azabicyclo[2.2.2]octan-3-one: A 1000-mL round-bottom flask was placed quinuclidin-3-one (50.00 g, 399.45 mmol). This was followed by the addition of K₂CO₃ (55.21 g, 399.45 mmol, 1.00 equiv), H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. The mixture was dried over anhydrous sodium sulfate and concentrated. The residue was applied onto a silica gel column with ethyl acetate/petroleum ether (0:1-1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of bis((2-(methoxymethyl)-3-oxoquinuclidin-2-yl)methyl) ((1R,3S)-cyclohexane-1,3-diyl)dicarbamate: A 50 ml round bottom flask was placed 2-(hydroxymethyl)-2-(methoxymethyl)-1-azabicyclo[2.2.2]octan-3-one (300 mg, 1.5 mmol), CDI (317 mg, 1.95 mmol) and DCE (10 ml). The resulting solution was stirred for 4 h at 50° C. (1R,3S)-cyclohexane-1,3-diamine (103 mg, 0.9 mmol) was added and the mixture was stirred for 48 h at 50° C. The resulting mixture was concentrated under reduced pressure. The crude product was purified by Prep-HPLC with the following conditions (Column: Kinetex EVO C18 Column, 21.2*150, Sum; Mobile Phase A: Water (0.05% NH₃H₂O), Mobile Phase B: ACN; Flow rate: 20 mL/min; Gradient: 13% B to 26% B in 8 min, 26% B; Wave Length: 220 NM nm; RT1 (min): 7.6;) to afford bis((2-(methoxymethyl)-3-oxoquinuclidin-2-yl)methyl) ((1R,3S)-cyclohexane-1,3-diyl)dicarbamate (20 mg) as a white solid. LC-MS: (ES, m/z): M+1: 565. ¹H NMR (300 MHz, Chloroform-d) δ 4.99-4.64 (m, 2H), 4.49 (d, J=11.7 Hz, 2H), 4.30 (d, J=11.7 Hz, 2H), 3.72 (s, 4H), 3.61-3.48 (m, 2H), 3.48-3.25 (m, 10H), 3.04-2.81 (m, 4H), 2.43 (q, J=3.1 Hz, 2H), 2.20-1.95 (m, 10H), 1.90-1.75 (m, 4H), 1.20-0.85 (m, 2H).

### Example 14: Preparation of 2-(methoxymethyl)-2-([[2-(methoxymethyl)-3-oxo-1-azabicyclo[2.2.2]octan-2-yl]methoxy]methyl)-1-azabicyclo[2.2.2]octan-3-one

Synthesis of 2-(hydroxymethyl)-2-(methoxymethyl)-1-azabicyclo[2.2.2]octan-3-one: Into a 1000-mL round-bottom flask, was placed 3-Quinuclidinone hydrochloride (50 g, 310.56 mmol, 1.00 equiv), H₂O (200.00 mL), MeOH (600.00 mL). This was followed by the addition of K₂CO₃ (50 g, 362.32 mmol, 1.17 equiv), 37% CH₂O (105 mL). The resulting solution was stirred for 5 h at 75 degrees C. Then the resulting solution was stirred overnight at room temperature. The resulting solution was adjusted PH=12 with 2N NaOH, extracted with 3×500 mL of dichloromethane and concentrated. The residue was applied onto a silica gel column with ethyl acetate/petroleum ether (1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200; H-NMR (300 MHz, Chloroform-d, ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of 2-(iodomethyl)-2-(methoxymethyl)-1-azabicyclo[2.2.2]octan-3-one: Into a 250-mL round-bottom flask, was placed 2-(hydroxymethyl)-2-(methoxymethyl)-1-azabicyclo[2.2.2]octan-3-one (2.00 g, 10.038 mmol, 1.00 equiv), DCM (30.00 mL), PPh₃ (3.42 g, 13.039 mmol, 1.30 equiv), 12 (3.32 g, 13.081 mmol, 1.30 equiv), imidazole (923.00 mg, 13.558 mmol, 1.35 equiv). The resulting solution was stirred overnight at room temperature. The resulting mixture was concentrated under vacuum. The residue was applied onto a silica gel column with ethyl acetate/petroleum ether (1:3). This resulted in 1.2 g (38.67%) of 2-(iodomethyl)-2-(methoxymethyl)-1-azabicyclo[2.2.2]octan-3-one as yellow oil. LC-MS: (ES, m/z): M+1: 310; H-NMR (300 MHz, Chloroform-d, ppm) δ 3.77-3.50 (m, 3H), 3.45-3.28 (m, 5H), 3.15 (ddt, J=14.5, 9.3, 4.1 Hz, 1H), 2.93 (dddd, J=31.6, 15.4, 10.2, 6.2 Hz, 2H), 2.58-2.44 (m, 1H), 2.05 (ddddt, J=22.3, 18.7, 13.2, 5.4, 3.1 Hz, 4H).

Synthesis of 2-(methoxymethyl)-2-([[2-(methoxymethyl)-3-oxo-1-azabicyclo[2.2.2]octan-2-yl]methoxy]methyl)-1-azabicyclo[2.2.2]octan-3-one: Into a 100-mL 3-necked round-bottom flask purged and maintained with an inert atmosphere of nitrogen, was placed 2-(hydroxymethyl)-2-(methoxymethyl)-1-azabicyclo[2.2.2]octan-3-one (400.00 mg, 2.008 mmol, 1.00 equiv), DCM (20.00 mL). This was followed by the addition of 2-(iodomethyl)-2-(methoxymethyl)-1-azabicyclo[2.2.2]octan-3-one (807.00 mg, 2.610 mmol, 1.30 equiv) at −78 degrees C. To this was added 2,6-di-tert-butyl-4-methylpyridine (935.00 mg, 4.553 mmol, 2.27 equiv) at −78 degrees C., then followed by AgSO₃CF₃ (1.14 g, 4.453 mmol, 2.22 equiv) at −78 degrees C. The resulting solution was stirred overnight at room temperature. The solids were filtered out. The resulting mixture was concentrated under vacuum. The residue was applied onto a silica gel column with dichloromethane/methanol (32:1). The crude product was purified by Prep-HPLC with the following conditions (2 #SHIMADZU (HPLC-01)): Column, X Bridge Shield RP18 OBD Column, 5 um, 19*150 mm; mobile phase, Water (0.05% NH₃.H₂O) and ACN (14% Phase B up to 36% in 7 min); Detector, UV. This resulted in 21 mg (2.75%) of 2-(methoxymethyl)-2-([[2-(methoxymethyl)-3-oxo-1-azabicyclo[2.2.2]octan-2-yl]methoxy]methyl)-1-azabicyclo[2.2.2]octan-3-one as a white solid. LC-MS: (ES, m/z): M+1: 381; H-NMR (300 MHz, Chloroform-d, ppm) δ 4.00 (dd, J=44.8, 10.0 Hz, 1H), 3.75-3.37 (m, 8H), 3.34 (s, 6H), 3.18-2.73 (m, 7H), 2.58-2.32 (m, 2H), 2.14-1.59 (m, 8H).

### Example 15: Preparation of (1R,2R,4S,6R)-2-(hydroxymethyl)-2-(methoxymethyl)-6-methylquinuclidin-3-one

Synthesis of ethyl 2-methylisonicotinate Into a 2000 mL 3-necked round-bottom flask were added 3-methylpyridine-4-carboxylic acid (40.00 g, 291.676 mmol, 1.00 equiv), EtOH (600.00 mL) and H2SO₄ (57.22 g, 583.352 mmol, 2 equiv) at room temperature. The resulting mixture was stirred for 16 h at 90 degrees C. The mixture was allowed to cool down to room temperature. The resulting mixture was concentrated and then diluted with EtOAc (600 mL). The mixture/residue was neutralized to pH 7 with NH₃. H₂O. The resulting mixture was extracted with EtOAc (2×500 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EtOAc (4:1) to afford ethyl 2-methylisonicotinate (45 g, 93.40%) as a yellow oil. LC-MS: (ES, m/z): M+1: 166.

Synthesis of 1-(2-ethoxy-2-oxoethyl)-4-(ethoxycarbonyl)-2-methylpyridin-1-ium: Into a 500 mL round-bottom flask were added ethyl 2-methylisonicotinate (45.00 g, 272.410 mmol, 1.00 equiv), EtOH (500 mL) and ethyl bromoacetate (68.24 g, 408.615 mmol, 1.50 equiv) at room temperature. The resulting mixture was stirred for 16 h at 80 degrees C. The resulting mixture was used in the next step directly without further purification.

Synthesis of ethyl 1-(2-ethoxy-2-oxoethyl)-2-methylpiperidine-4-carboxylate: Into a 2000 mL pressure tank reactor were added reaction mixture from last step, EtOH (600 mL) and Pd/C (8.00 g, 3.759 mmol, 0.05 equiv, 10%) at room temperature. The resulting mixture was stirred for 16 h at 80 degrees C. under hydrogen atmosphere (30 atm). The mixture was allowed to cool down to room temperature. The resulting mixture was filtered, the filter cake was washed with EtOH (2×200 mL). The filtrate was concentrated under reduced pressure. The residue was purified by silica gel column chromatography, eluted with PE/EtOAc (1:1) to afford ethyl 1-(2-ethoxy-2-oxoethyl)-2-methylpiperidine-4-carboxylate(cis) (27 g, 38.51%, 2 step) as a yellow oil. LC-MS: (ES, m/z): M+1: 258.

Synthesis of ethyl-6-methyl-3-oxoquinuclidine-2-carboxylate: Into a 1000 mL round-bottom flask were added ethyl 1-(2-ethoxy-2-oxoethyl)-2-methylpiperidine-4-carboxylate(cis) (27.00 g, 104.92 mmol, 1.00 equiv), Toluene (500.00 mL) and t-BuOK (35.32 g, 314.76 mmol, 3.00 equiv) at room temperature. The resulting mixture was stirred for 2 h at 110 degrees C. under nitrogen atmosphere. The mixture/residue was neutralized to pH 7 with HCl (4M). The resulting mixture was concentrated under reduced pressure. The residue was purified by silica gel column chromatography, eluted with CH₂Cl₂/MeOH (10:1) to afford ethyl-6-methyl-3-oxoquinuclidine-2-carboxylate(racemate) (4.8 g, 21.65%) as a colorless oil. LC-MS: (ES, m/z): M+1: 212.

Synthesis of (1S,4S,6R and 1R,4R,6S)-6-methylquinuclidin-3-one hydrogen chloride (racemate): Into a 50 mL round-bottom flask were added ethyl-6-methyl-3-oxoquinuclidine-2-carboxylate (4.8 g, 22.72 mmol, 1.00 equiv) and HCl (20.00 mL) at room temperature. The resulting mixture was stirred for 4 h at 100 degrees C. The resulting mixture was concentrated under reduced pressure. The resulting mixture was diluted with Et₂O (30 mL). The precipitated solids were collected by filtration and washed with Et₂O (2×10 mL). Then (1S,4S,6R and 1R,4R,6S)-6-methylquinuclidin-3-one hydrogen chloride (3.7 g, 92.73%) (racemate) was afforded as a white solid. LC-MS: (ES, m/z): M+1: 140.

Synthesis of mixtures of (1S,2S,4R,6S and 1R,2R,4S,6R)-2-(hydroxymethyl)-2-(methoxymethyl)-6-methylquinuclidin-3-one (racemate) and (1S,2R,4R,6S and 1R,2S,4S,6R)-2-(hydroxymethyl)-2-(methoxymethyl)-6-methylquinuclidin-3-one (racemate): To a stirred solution/mixture of (1S,4S,6R and 1R,4R,6S)-6-methylquinuclidin-3-one hydrogen chloride (racemate) (3.7 g, 26.58 mmol, 1.00 equiv) and K₂CO₃ (18.37 g, 132.90 mmol, 5.00 equiv) in MeOH (60.00 mL), H₂O (20.00 mL) was added HCHO (21.57 g, 265.81 mmol, 10.00 equiv, 37%) at room temperature. The resulting mixture was stirred for 2 h at 70 degrees C. The resulting mixture was diluted with DCM (200 mL). The resulting mixture was extracted with DCM (2×200 mL). The combined organic layers were washed with brine (1×200 mL), dried over anhydrous Na₂SO₄. After filtration, the filtrate was concentrated under reduced pressure. The residue was purified by silica gel column chromatography, eluted with DCM/MeOH (10:1) to afford a Synthesis of a mixture of (1S,2S,4R,6S and 1R,2R,4S,6R)-2-(hydroxymethyl)-2-(methoxymethyl)-6-methylquinuclidin-3-one (racemate, major peak) and (1S,2R,4R,6S and 1R,2S,4S,6R)-2-(hydroxymethyl)-2-(methoxymethyl)-6-methylquinuclidin-3-one (racemate, minor peak) (600 mg, ration=9:1) as a colorless oil. LC-MS (ES, m/z): M+1: 214.

Synthesis of (1R,2R,4S,6R)-2-(hydroxymethyl)-2-(methoxymethyl)-6-methylquinuclidin-3-one: 300 mg of (1S,2S,4R,6S and 1R,2R,4S,6R)-2-(hydroxymethyl)-2-(methoxymethyl)-6-methylquinuclidin-3-one (racemate, purity=90%) was separated by SFC with the following conditions: Column, 5: IG, 4.6*100 mm, 3 um; mobile phase A: Hexane; mobile phase B: MeOH (20 mM NH₃); Flow rate: 3.0 ml/min; Gradient: 10% B to 30% B in 4 min, RT: 1.416 min; Detector, 210 nm. This resulted in (1R,2R,4S,6R)-2-(hydroxymethyl)-2-(methoxymethyl)-6-methylquinuclidin-3-one (80 mg, 26.67%) as a semi solid. LC-MS-: (ES, m/z): M+1: 214. ANAL_SFC: Column Name: IG 100×4.6 mm 3.0 um, Co Solvent: CO₂: 30% MeOH (20 mM NH₃). Total Flow: 3.0000 mL/min. Isogradient: 4 min, RT=1.416 min. ¹H NMR (300 MHz, Chloroform-d) δ 4.05-3.90 (m, 2H), 3.88 (d, J=4.8 Hz, 2H), 3.58 (q, J=7.7 Hz, 1H), 3.39 (s, 3H), 3.30 (td, J=9.7, 9.3, 5.2 Hz, 1H), 3.16 (tq, J=14.2, 4.7 Hz, 1H), 3.06 (s, 1H), 2.36 (t, J=3.1 Hz, 1H), 2.25 (dd, J=13.2, 10.0 Hz, 1H), 1.98 (tt, J=9.5, 7.4 Hz, 2H), 1.54 (ddd, J=13.3, 6.6, 2.4 Hz, 1H), 1.39 (d, J=6.8 Hz, 3H).

### Example 16: Preparation of (1S,2S,4R,6S)-2-(hydroxymethyl)-2-(methoxymethyl)-6-methylquinuclidin-3-one

Synthesis of ethyl 2-methylisonicotinate: Into a 2000 mL 3-necked round-bottom flask were added 3-methylpyridine-4-carboxylic acid (40.00 g, 291.676 mmol, 1.00 equiv), EtOH (600.00 mL) and H2SO₄ (57.22 g, 583.352 mmol, 2 equiv) at room temperature. The resulting mixture was stirred for 16 h at 90 degrees C. The mixture was allowed to cool down to room temperature. The resulting mixture was concentrated and then diluted with EtOAc (600 mL). The mixture/residue was neutralized to pH 7 with NH₃. H₂O. The resulting mixture was extracted with EtOAc (2×500 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EtOAc (4:1) to afford ethyl 2-methylisonicotinate (45 g, 93.40%) as a yellow oil. LC-MS: (ES, m/z): M+1: 166.

Synthesis of 1-(2-ethoxy-2-oxoethyl)-4-(ethoxycarbonyl)-2-methylpyridin-1-ium: Into a 500 mL round-bottom flask were added ethyl 2-methylisonicotinate (45.00 g, 272.410 mmol, 1.00 equiv), EtOH (500 mL) and ethyl bromoacetate (68.24 g, 408.615 mmol, 1.50 equiv) at room temperature. The resulting mixture was stirred for 16 h at 80 degrees C. The resulting mixture was used in the next step directly without further purification.

Synthesis of ethyl 1-(2-ethoxy-2-oxoethyl)-2-methylpiperidine-4-carboxylate: Into a 2000 mL pressure tank reactor were added reaction mixture from last step, EtOH (600 mL) and Pd/C (8.00 g, 3.759 mmol, 0.05 equiv, 10%) at room temperature. The resulting mixture was stirred for 16 h at 80 degrees C. under hydrogen atmosphere (30 atm). The mixture was allowed to cool down to room temperature. The resulting mixture was filtered, the filter cake was washed with EtOH (2×200 mL). The filtrate was concentrated under reduced pressure. The residue was purified by silica gel column chromatography, eluted with PE/EtOAc (1:1) to afford ethyl 1-(2-ethoxy-2-oxoethyl)-2-methylpiperidine-4-carboxylate(cis) (27 g, 38.51%, 2 step) as a yellow oil. LC-MS: (ES, m/z): M+1: 258.

Synthesis of ethyl-6-methyl-3-oxoquinuclidine-2-carboxylate: Into a 1000 mL round-bottom flask were added ethyl 1-(2-ethoxy-2-oxoethyl)-2-methylpiperidine-4-carboxylate(cis) (27.00 g, 104.92 mmol, 1.00 equiv), Toluene (500.00 mL) and t-BuOK (35.32 g, 314.76 mmol, 3.00 equiv) at room temperature. The resulting mixture was stirred for 2 h at 110 degrees C. under nitrogen atmosphere. The mixture/residue was neutralized to pH 7 with HCl (4M). The resulting mixture was concentrated under reduced pressure. The residue was purified by silica gel column chromatography, eluted with CH₂Cl₂/MeOH (10:1) to afford ethyl-6-methyl-3-oxoquinuclidine-2-carboxylate(racemate) (4.8 g, 21.65%) as a colorless oil. LC-MS: (ES, m/z): M+1: 212.

Synthesis of (1S,4S,6R and 1R,4R,6S)-6-methylquinuclidin-3-one hydrogen chloride (racemate): Into a 50 mL round-bottom flask were added ethyl-6-methyl-3-oxoquinuclidine-2-carboxylate (4.8 g, 22.72 mmol, 1.00 equiv) and HCl (20.00 mL) at room temperature. The resulting mixture was stirred for 4 h at 100 degrees C. The resulting mixture was concentrated under reduced pressure. The resulting mixture was diluted with Et₂O (30 mL). The precipitated solids were collected by filtration and washed with Et₂O (2×10 mL). Then (1S,4S,6R and 1R,4R,6S)-6-methylquinuclidin-3-one hydrogen chloride (3.7 g, 92.73%) (racemate) was afforded as a white solid. LC-MS: (ES, m/z): M+1: 140.

Synthesis of a mixture of (1S,2S,4R,6S and 1R,2R,4S,6R)-2-(hydroxymethyl)-2-(methoxymethyl)-6-methylquinuclidin-3-one (racemate) and (1S,2R,4R,6S and 1R,2S,4S,6R)-2-(hydroxymethyl)-2-(methoxymethyl)-6-methylquinuclidin-3-one (racemate): To a stirred solution/mixture of (1S,4S,6R and 1R,4R,6S)-6-methylquinuclidin-3-one hydrogen chloride (racemate) (3.7 g, 26.58 mmol, 1.00 equiv) and K₂CO₃ (18.37 g, 132.90 mmol, 5.00 equiv) in MeOH (60.00 mL), H₂O (20.00 mL) was added HCHO (21.57 g, 265.81 mmol, 10.00 equiv, 37%) at room temperature. The resulting mixture was stirred for 2 h at 70 degrees C. The resulting mixture was diluted with DCM (200 mL). The resulting mixture was extracted with DCM (2×200 mL). The combined organic layers were washed with brine (1×200 mL), dried over anhydrous Na₂SO₄. After filtration, the filtrate was concentrated under reduced pressure. The residue was purified by silica gel column chromatography, eluted with DCM/MeOH (10:1) to afford a Synthesis of a mixture of (1S,2S,4R,6S and 1R,2R,4S,6R)-2-(hydroxymethyl)-2-(methoxymethyl)-6-methylquinuclidin-3-one (racemate, major peak) and (1S,2R,4R,6S and 1R,2S,4S,6R)-2-(hydroxymethyl)-2-(methoxymethyl)-6-methylquinuclidin-3-one (racemate, minor peak) (600 mg, ration=9:1) as a colorless oil. LC-MS (ES, m/z): M+1: 214.

Synthesis of (1S,2S,4R,6S)-2-(hydroxymethyl)-2-(methoxymethyl)-6-methylquinuclidin-3-one: 300 mg of (1S,2S,4R,6S and 1R,2R,4S,6R)-2-(hydroxymethyl)-2-(methoxymethyl)-6-methylquinuclidin-3-one (racemate, purity=90%) was separated by SFC with the following conditions: Column, 5: IG, 4.6*100 mm, 3 um; mobile phase A: Hexane; mobile phase B: MeOH (20 mM NH₃); Flow rate: 3.0 ml/min; Gradient: 10% B to 30% B in 4 min, RT: 1.656 min; Detector, 210 nm. This resulted in (1S,2S,4R,6S)-2-(hydroxymethyl)-2-(methoxymethyl)-6-methylquinuclidin-3-one (75 mg, 25%) as a semi solid. LC-MS: (ES, m/z): M+1: 214. ANAL_SFC: Column Name: IG 100×4.6 mm 3.0 um, Co Solvent: CO₂: 30% MeOH (20 mM NH₃). Total Flow: 3.0000 mL/min. Isogradient: 4 min, RT=1.656 min. ¹H NMR (300 MHz, Chloroform-d) δ 4.06-3.90 (m, 2H), 3.90-3.71 (m, 2H), 3.61-3.45 (m, 1H), 3.40 (d, J=4.1 Hz, 3H), 3.29 (ddd, J=16.6, 10.0, 7.0 Hz, 1H), 3.21-3.05 (m, 1H), 3.00 (s, 1H), 2.35 (p, J=2.9 Hz, 1H), 2.31-2.17 (m, 1H), 2.08-1.88 (m, 2H), 1.66-1.47 (m, 1H), 1.42-1.24 (m, 3H).

### Example 17: Preparation of (1S,2S,4R,5S)-2-(hydroxymethyl)-2-(methoxymethyl)-5-methylquinuclidin-3-one (Assumed), (1S,2R,4R)-2-(hydroxymethyl)-2-(methoxymethyl)-5-methylquinuclidin-3-one (Assumed) and (1R,2R,4S,5R)-2-(hydroxymethyl)-2-(methoxymethyl)-5-methylquinuclidin-3-one (Assumed)

Synthesis of ethyl 3-methylisonicotinate: Into a 1000 mL 3-necked round-bottom flask were added 3-methylpyridine-4-carboxylic acid (20.00 g, 145.838 mmol, 1.00 equiv), EtOH (300.00 mL) and H2SO₄ (28.61 g, 291.677 mmol, 2 equiv) at room temperature. The resulting mixture was stirred for 16 h at 90° C. The mixture was allowed to cool down to room temperature. The resulting mixture was concentrated and then diluted with EtOAc (300 mL). The mixture/residue was neutralized to pH 7 with NH₃H₂O. The resulting mixture was extracted with EtOAc (2×300 mL). The combined organic layers were washed with brine (1×300 mL), dried over anhydrous Na₂SO₄. After filtration, the filtrate was concentrated under reduced pressure. The residue was purified by silica gel column chromatography, eluted with PE/EtOAc (4:1) to afford ethyl 3-methylpyridine-4-carboxylate (20 g, 83.02%) as a yellow oil. LC-MS-PH-PHNW-2153-2: (ES, m/z): M+1: 166

Synthesis of 1-(2-ethoxy-2-oxoethyl)-4-(ethoxycarbonyl)-3-methylpyridin-1-ium: Into a 500 mL round-bottom flask were added ethyl 3-methylpyridine-4-carboxylate (20.00 g, 121.071 mmol, 1.00 equiv), EtOH (250 mL) and ethyl bromoacetate (30.33 g, 181.606 mmol, 1.50 equiv) at room temperature. The resulting mixture was stirred for 16 h at 90° C. The resulting mixture was used in the next step directly without further purification.

Synthesis of ethyl 1-(2-ethoxy-2-oxoethyl)-3-methylpiperidine-4-carboxylate: Into a 1000 mL pressure tank reactor were added reaction mixture from last step, EtOH (200 mL) and Pd/C (4.00 g, 3.759 mmol, 0.05 equiv, 10%) at room temperature. The resulting mixture was stirred for 16 h at 80° C. under hydrogen atmosphere (30 atm). The mixture was allowed to cool down to room temperature. The resulting mixture was filtered; the filter cake was washed with EtOH (2×100 mL). The filtrate was concentrated under reduced pressure. The residue was purified by silica gel column chromatography, eluted with PE/EtOAc (1:1) to afford ethyl 1-(2-ethoxy-2-oxoethyl)-3-methylpiperidine-4-carboxylate (12 g, 58.82%) as a yellow oil. LC-MS: (ES, m/z): M+1: 258

Synthesis of ethyl 3-methyl-5-oxoquinuclidine-4-carboxylate: Into a 500 mL round-bottom flask were added ethyl 1-(2-ethoxy-2-oxoethyl)-3-methylpiperidine-4-carboxylate (12.00 g, 46.633 mmol, 1.00 equiv), Toluene (300.00 mL) and t-BuOK (15.70 g, 139.914 mmol, 3.00 equiv) at room temperature. The resulting mixture was stirred for 2 h at 110 degrees C. under nitrogen atmosphere. The mixture/residue was acidified/basified/neutralized to pH 7 with HCl (4M). The resulting mixture was concentrated under reduced pressure. The residue was purified by silica gel column chromatography, eluted with DCM/MeOH (10:1) to afford ethyl 3-methyl-5-oxo-1-azabicyclo[2.2.2]octane-4-carboxylate (2.7 g, 27.41%) as a colorless oil. LC-MS: (ES, m/z): M+1: 212 Synthesis of (1R,4S,5R)-5-methylquinuclidin-3-one (racemate): Into a 50 mL round-bottom flask were added ethyl 3-methyl-5-oxo-1-azabicyclo[2.2.2]octane-4-carboxylate (2.70 g, 12.780 mmol, 1.00 equiv) and HCl (13.00 mL, 189.789 mmol, 17.82 equiv) at room temperature. The resulting mixture was stirred for 4 h at 100 degrees C. The resulting mixture was concentrated under reduced pressure. The resulting mixture was diluted with Et₂O (20 mL). The precipitated solids were collected by filtration and washed with Et₂O (2×10 mL). Then 5-methyl-1-azabicyclo[2.2.2]octan-3-one hydrochloride(racemate) (1.8 g, 80.18%) was afforded as a white solid. LC-MS: (ES, m/z): M+1: 140

Synthesis of (1R,2R,4S,5R)-2-(hydroxymethyl)-2-(methoxymethyl)-5-methylquinuclidin-3-one (racemate): To a stirred solution/mixture of (1R,4S,5R)-5-methyl-1-azabicyclo[2.2.2]octan-3-one hydrochloride (1.80 g, 10.247 mmol, 1.00 equiv) and K₂CO₃ (7.08 g, 51.235 mmol, 5.00 equiv) in MeOH (30.00 mL), H₂O (10.00 mL) was added HCHO (8.32 g, 102.470 mmol, 10.00 equiv, 37%) at room temperature. The resulting mixture was stirred for 2 h at 70° C. The resulting mixture was diluted with DCM (100 mL). The resulting mixture was extracted with DCM (2×100 mL). The combined organic layers were washed with brine (1×100 mL), dried over anhydrous Na₂SO₄. After filtration, the filtrate was concentrated under reduced pressure. The residue was purified by silica gel column chromatography, eluted with DCM/MeOH (10:1) to afford (1R,2R,4S,5R)-2-(hydroxymethyl)-2-(methoxymethyl)-5-methylquinuclidin-3-one (racemate) (500 mg, 22.88%) as a white solid. LC-MS: (ES, m/z): M+1: 214

Synthesis of (1S,2S,4R,5S)-2-(hydroxymethyl)-2-(methoxymethyl)-5-methylquinuclidin-3-one (Assumed): 200 mg of (1R,2R,4S,5R)-2-(hydroxymethyl)-2-(methoxymethyl)-5-methylquinuclidin-3-one (racemate) was separated by SFC with the following conditions: Column, CHIRALPAK IG-3, 100*4.6 mm, 3 um IG30CS-UL011; mobile phase A: n-Hexane (0.1% DEA); mobile phase B: Ethanol; Flow rate: 1.0 ml/min; Gradient: 30% B (10 min, RT: 5.281 min); Detector, 220 nm. This resulted in (1S,2S,4R,5S)-2-(hydroxymethyl)-2-(methoxymethyl)-5-methylquinuclidin-3-one (Assumed) (60 mg, 30%) as a white solid. LC-MS: (ES, m/z): M+1: 214. H-NMR: ¹H NMR (300 MHz, Chloroform-d) δ 3.98 (d, J=11.8 Hz, 1H), 3.82 (s, 3H), 3.56 (ddd, J=13.5, 9.9, 2.9 Hz, 1H), 3.42 (s, 3H), 3.30 (dd, J=14.2, 3.9 Hz, 1H), 2.93 (ddd, J=14.7, 10.2, 5.8 Hz, 1H), 2.40 (dd, J=14.3, 6.6 Hz, 1H), 2.31-2.18 (m, 3H), 1.96-1.82 (m, 1H), 1.17 (d, J=6.7 Hz, 3H).

Synthesis of (1S,2R,4R)-2-(hydroxymethyl)-2-(methoxymethyl)-5-methylquinuclidin-3-one (Assumed): 200 mg of (1R,2R,4S,5R)-2-(hydroxymethyl)-2-(methoxymethyl)-5-methylquinuclidin-3-one (racemate) was separated by SFC with the following conditions: Column, CHIRALPAK AY-3, 50*4.6 mm, 3 um AY30CC-SK001; mobile phase A: n-Hexane (0.1% DEA); mobile phase B: Ethanol; Flow rate: 1.0 ml/min; Gradient: 20% B (5 min, RT: 1.675 min, 1.908 min); Detector, 220 nm. This resulted in (1S,2R,4R)-2-(hydroxymethyl)-2-(methoxymethyl)-5-methylquinuclidin-3-one (Assumed) (20 mg, 10%) as a white solid. LC-MS: (ES, m/z): M+1: 214. H-NMR: ¹H NMR (300 MHz, Chloroform-d) δ 4.01 (d, J=11.8 Hz, 1H), 3.89-3.74 (m, 3H), 3.63-3.48 (m, 1H), 3.42 (s, 3H), 3.30 (dt, J=10.9, 7.1 Hz, 1H), 2.90 (ddd, J=14.3, 10.7, 6.4 Hz, 1H), 2.85 (s, 1H), 2.43 (dd, J=14.2, 6.5 Hz, 1H), 2.36-2.19 (m, 3H), 1.93-1.76 (m, 1H), 1.17 (d, J=6.8 Hz, 3H).

Synthesis of (1R,2R,4S,5R)-2-(hydroxymethyl)-2-(methoxymethyl)-5-methylquinuclidin-3-one (Assumed): 200 mg of (1R,2R,4S,5R)-2-(hydroxymethyl)-2-(methoxymethyl)-5-methylquinuclidin-3-one (racemate) was separated by SFC with the following conditions: Column, CHIRALPAK AY-3, 50*4.6 mm, 3 um AY30CC-SK001; mobile phase A: n-Hexane (0.1% DEA); mobile phase B: Ethanol; Flow rate: 1.0 ml/min; Gradient: 20% B (5 min, RT: 1.325 min); Detector, 220 nm. This resulted in (1R,2R,4S,5R)-2-(hydroxymethyl)-2-(methoxymethyl)-5-methylquinuclidin-3-one (Assumed) (40 mg, 20%) as a white solid. LC-MS: (ES, m/z): M+1: 214. H-NMR: ¹H NMR (300 MHz, Chloroform-d) δ 3.98 (d, J=11.7 Hz, 1H), 3.83 (s, 1H), 3.82 (s, 2H), 3.63-3.47 (m, 1H), 3.41 (s, 3H), 3.39-3.22 (m, 1H), 3.00-2.84 (m, 1H), 2.84 (s, 1H), 2.46-2.24 (m, 1H), 2.29-2.16 (m, 2H), 1.95-1.78 (m, 1H), 1.17 (d, J=6.7 Hz, 3H).

### Example 18: Preparation of (2-(methoxymethyl)-3-oxoquinuclidin-2-yl)methyl (((2-(methoxymethyl)-3-oxoquinuclidin-2-yl)methoxy)carbonyl)-L-valinate

Synthesis of 2-(hydroxymethyl)-2-(methoxymethyl)-1-azabicyclo[2.2.2]octan-3-one: Into a 1000-mL round-bottom flask, was placed quinuclidin-3-one (50.00 g, 399.45 mmol). This was followed by the addition of K₂CO₃ (55.21 g, 399.45 mmol, 1.00 equiv), H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. The mixture was dried over anhydrous sodium sulfate and concentrated. The residue was applied onto a silica gel column with ethyl acetate/petroleum ether (0:1-1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of (2-(methoxymethyl)-3-oxoquinuclidin-2-yl)methyl (tert-butoxycarbonyl)-L-valinate: Into a 50-mL round-bottom flask was placed 2-(hydroxymethyl)-2-(methoxymethyl)-1-azabicyclo[2.2.2]octan-3-one (500.0 mg, 2.51 mmol, 1.0 equiv). This was followed by the addition of (tert-butoxycarbonyl)-L-valine (545.2 mg, 2.51 mmol, 1.00 equiv), DCC (932.1 mg, 4.52 mmol), DMAP (61.3 mg, 0.51 mmol), DCM (15.0 mL). The resulting solution was stirred for 6 h at room temperature. The resulting solution was extracted with 3×10 mL of dichloromethane. The organic layer was dried over anhydrous sodium sulfate and concentrated. The residue was applied onto a silica gel column with DCM/MeOH (10;1). This resulted in 420 mg (42.00%) of (2-(methoxymethyl)-3-oxoquinuclidin-2-yl)methyl (tert-butoxycarbonyl)-L-valinate as a white solid. LC-MS: (ES, m/z): M+1: 399

Synthesis of (2-(methoxymethyl)-3-oxoquinuclidin-2-yl)methyl L-valinate: Into a 25-mL round-bottom flask was placed (2-(methoxymethyl)-3-oxoquinuclidin-2-yl)methyl (tert-butoxycarbonyl)-L-valinate (420 mg, 1.0 equiv, 1.05 mmol). This was followed by the addition of HCl in Dioxane (4N, 10.0 mL). The resulting solution was stirred for 2 h at room temperature. The resulting solution was concentrated. This resulted in 370 mg (94.8%, HCl salt) of (2-(methoxymethyl)-3-oxoquinuclidin-2-yl)methyl L-valinate as a white solid. LC-MS: (ES, m/z): M+1: 299

Synthesis of (2-(methoxymethyl)-3-oxoquinuclidin-2-yl)methyl (((2-(methoxymethyl)-3-oxoquinuclidin-2-yl)methoxy)carbonyl)-L-valinate: Into a 25 mL round-bottom flask were added 2-(hydroxymethyl)-2-(methoxymethyl)-1-azabicyclo[2.2.2]octan-3-one (150.00 mg, 0.753 mmol, 1.00 equiv) DCE (3.00 mL) and CDI (146.48 mg, 0.903 mmol, 1.2 equiv) at room temperature. The resulting mixture was stirred for 3 h at 50 degrees C. To the above mixture was added [2-(methoxymethyl)-3-oxo-1-azabicyclo[2.2.2]octan-2-yl]methyl (2S)-2-amino-3-methylbutanoate (224.63 mg, 0.753 mmol, 1 equiv) and TEA (152.36 mg, 1.506 mmol, 2 equiv) at 50° C. The resulting mixture was stirred for additional 5 h at 50° C. The reaction was quenched with Water at room temperature. The resulting mixture was extracted with CH₂Cl₂ (2×10 mL). The combined organic layers were washed with brine (1×10 mL), dried over anhydrous Na₂SO₄. After filtration, the filtrate was concentrated under reduced pressure. The crude product (mg) was purified by Prep-HPLC with the following conditions (Column: Atlantis Prep T3 OBD Column, 19*150 mm Sum; Mobile Phase A: Water (0.05% TFA), Mobile Phase B: ACN; Flow rate: 20 mL/min; Gradient: 10% B to 30% B in 7 min, 30% B; Wave Length: 202 nm;) and ACN (42% PhaseB up to 56% in 7 min); Detector, 254 nm.) to afford [2-(methoxymethyl)-3-oxo-1-azabicyclo[2.2.2]octan-2-yl]methyl (2S)-2-[([[2-(methoxymethyl)-3-oxo-1-azabicyclo[2.2.2]octan-2-yl]methoxy]carbonyl)amino]-3-methylbutanoate (40 mg, 10.15%) as a white solid. LC-MS: (ES, m/z): M+1: 524. ¹H NMR (400 MHz, DMSO-d₆) δ 8.06 (dd, J=19.3, 8.7 Hz, 1H), 4.84-4.33 (m, 4H), 4.06 (dd, J=16.3, 7.9 Hz, 2H), 3.98-3.80 (m, 4H), 3.62 (d, J=10.7 Hz, 4H), 3.32 (d, J=10.7 Hz, 6H), 2.69 (d, J=8.6 Hz, 2H), 2.34-2.00 (m, 9H), 0.90 (t, J=6.5 Hz, 6H).

### Example 19: Preparation of bis((2-(methoxymethyl)-3-oxoquinuclidin-2-yl)methyl) terephthalate

Synthesis of 2-(hydroxymethyl)-2-(methoxymethyl)-1-azabicyclo[2.2.2]octan-3-one: Into a 1000-mL round-bottom flask, was placed quinuclidin-3-one (50.00 g, 399.45 mmol). This was followed by the addition of K₂CO₃ (55.21 g, 399.45 mmol, 1.00 equiv), H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. The mixture was dried over anhydrous sodium sulfate and concentrated. The residue was applied onto a silica gel column with ethyl acetate/petroleum ether (0:1-1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of bis((2-(methoxymethyl)-3-oxoquinuclidin-2-yl)methyl) terephthalate: To a stirred solution of 2-(hydroxymethyl)-2-(methoxymethyl)-1-azabicyclo[2.2.2]octan-3-one (150 mg, 0.753 mmol, 2.0 equiv) and terephthalic acid (75.04 mg, 0.452 mmol, 1.00 equiv) in DCM was added DCC (279.59 mg, 1.355 mmol, 3.6 equiv) and DMAP (18.39 mg, 0.151 mmol, 0.4 equiv) at room temperature. The resulting mixture was stirred for 3 h at room temperature. The resulting mixture was extracted with CH₂Cl₂ (2×10 mL). The combined organic layers were washed with brine (1×10 mL), dried over anhydrous Na₂SO₄. After filtration, the filtrate was concentrated under reduced pressure. The crude product was purified by Prep-HPLC with the following conditions (Column: Atlantis Prep T3 OBD Column, 19*150 mm Sum; Mobile Phase A: Water (0.05% TFA), Mobile Phase B: ACN; Flow rate: 20 mL/min; Gradient: 10% B to 30% B in 7 min, 30% B; Wave Length: 202 nm;) and ACN (42% PhaseB up to 56% in 7 min); Detector, 254 nm.) to afford bis((2-(methoxymethyl)-3-oxoquinuclidin-2-yl)methyl) terephthalate (70 mg, 29.32%) as a white solid. LC-MS: (ES, m/z): M+1: 529. ¹H NMR (300 MHz, Chloroform-d) δ 8.27 (s, 4H), 4.85 (d, J=3.4 Hz, 4H), 4.47 (d, J=11.2 Hz, 2H), 4.12 (s, 2H), 4.01 (d, J=11.2 Hz, 2H), 3.86 (s, 2H), 3.55 (s, 4H), 3.41 (s, 6H), 2.84 (s, 2H), 2.32 (d, J=32.6 Hz, 8H).

### Example 20: Preparation of (1S,5R,7S)-7-(hydroxymethyl)-7-(methoxymethyl)-1-azabicyclo[3.2.2]nonan-6-one & (1R,5S,7R)-7-(hydroxymethyl)-7-(methoxymethyl)-1-azabicyclo[3.2.2]nonan-6-one & (1S,5R,7R)-7-(hydroxymethyl)-7-(methoxymethyl)-1-azabicyclo[3.2.2]nonan-6-one & (1R,5S,7S)-7-(hydroxymethyl)-7-(methoxymethyl)-1-azabicyclo[3.2.2]nonan-6-one

Synthesis of tert-butyl 4-cyanoazepane-1-carboxylate: Into a 2000- mL round-bottom flask purged and maintained with an inert atmosphere of nitrogen, was placed tert-butyl 4-oxoazepane-1-carboxylate (40 g, 187.550 mmol, 1.00 equiv), DME (800 mL). This was followed by the addition of Tos-Mic (84.22 g, 431.365 mmol, 2.3 equiv), t-BuOK (73.66 g, 656.425 mmol, 3.5 equiv), t-BuOH (31.97 g, 431.365 mmol, 2.3 equiv) at 0 degrees C. The resulting solution was stirred for 16 h at room temperature. The reaction was then quenched by the addition of 800 mL of water. The resulting solution was extracted with 2×800 mL of ethyl acetate and washed with 2×800 ml of brine. The mixture was dried over anhydrous sodium sulfate and concentrated under vacuum. The residue was applied onto a silica gel column with ethyl acetate/petroleum ether (1:2). This resulted in 20 g (Y=47.54%) tert-butyl 4-cyanoazepane-1-carboxylate as a light yellow oil. 1H NMR (300 MHz, Chloroform-d, ppm) δ 3.66-3.35 (m, 4H), 2.86 (q, J=5.6, 5.0 Hz, 1H), 2.14-1.86 (m, 6H), 1.48 (d, J=2.4 Hz, 9H).

Synthesis of 1-(tert-butoxycarbonyl)azepane-4-carboxylic acid: Into a 500-mL round-bottom flask, was placed tert-butyl 4-cyanoazepane-1-carboxylate (20 g, 89.165 mmol, 1.00 equiv), H₂O (10% NaOH, 300 mL). The resulting solution was stirred for overnight at 100 degrees C. The mixture as acidified to pH 5-6 with HCl (1M). The resulting solution was extracted with 3×400 mL of ethyl acetate and washed with 2×400 ml of brine. The mixture was dried over anhydrous sodium sulfate and concentrated under vacuum. This resulted in 15 g crude 1-(tert-butoxycarbonyl) azepane-4-carboxylic acid as a light yellow oil. LC-MS: (ES, m/z): M-56+1: 188. ¹H NMR (300 MHz, DMSO-d₆, ppm) δ 12.12 (s, 1H), 3.50-3.37 (m, 1H), 3.29-3.14 (m, 3H), 2.37-2.29 (s, 2H), 1.99-1.74 (m, 3H) 1.68-1.47 (m, 2H), 1.41 (s, 9H).

Synthesis of tert-butyl 4-(methoxy(methyl)carbamoyl)azepane-1-carboxylate: Into a 500- mL round-bottom flask was placed 1-(tert-butoxycarbonyl)azepane-4-carboxylic acid (14 g, 57.541 mmol, 1.00 equiv), DCM (150 mL), CDI (9.33 g, 57.541 mmol, 1.0 equiv). The resulting solution was stirred for 1 h at room temperature. And then was followed by the addition of N,O-dimethylhydroxylamine (4.22 g, 69.049 mmol, 1.2 equiv), TEA (17.47 g, 172.623 mmol, 3.0 equiv). The resulting solution was stirred for 16 h at room temperature. The reaction was quenched by the addition of 200 mL of water. The resulting solution was extracted with 2×150 mL of dichloromethane. The mixture was washed with 2×200 ml of water and 2×200 ml of brine. The mixture was dried over anhydrous sodium sulfate and concentrated under vacuum. The residue was applied onto a silica gel column with ethyl acetate/petroleum ether (EA). This resulted in 12 g (Y=72.82%) tert-butyl 4-[methoxy(methyl)carbamoyl]azepane-1-carboxylateas a light yellow oil. LC-MS: (ES, m/z): M+1: 287. ¹H NMR (300 MHz, DMSO-d₆, ppm) δ 3.66 (d, J=1.4 Hz, 3H), 3.51-3.35 (m, 2H), 3.36-3.09 (m, 2H), 3.08 (s, 3H), 2.77-2.64 (m, 2H), 1.94-1.64 (m, 3H), 1.68-1.43 (m, 1H), 1.41 (s, 9H), 1.38-1.08 (m, 1H).

Synthesis of tert-butyl 4-acetylazepane-1-carboxylate: Into a 250- mL round-bottom flask purged and maintained with an inert atmosphere of nitrogen, was placed tert-butyl 4-[methoxy(methyl)carbamoyl]azepane-1-carboxylate (10 g, 34.965 mmol, 1.0 equiv), THF (100 mL). This was followed by the addition of CH₃MgBr (3M in 2-Methyltetrahydrofuran, 29 mL, 2.5 equiv) at 0 degrees C. The resulting solution was stirred for 2 h at 5 degrees C. The reaction was quenched by the addition of 100 mL of NH₄Cl (aq). The resulting solution was extracted with 2×100 mL of ethyl acetate and washed with 2×100 ml of brine. The mixture was dried over anhydrous sodium sulfate and concentrated under vacuum. The residue was applied onto a silica gel column with ethyl acetate/petroleum ether (EA). This resulted in 7.3 g (Y=86.69%) tert-butyl 4-acetylazepane-1-carboxylate as a light yellow oil. LC-MS: (ES, m/z): M-56+1: 186. ¹H NMR (300 MHz, DMSO-d6, ppm) δ 3.52-3.31 (m, 2H), 3.28-3.15 (m, 2H), 2.45 (dd, J=9.9, 3.5 Hz, 1H), 2.11 (s, 3H), 1.96-1.75 (m, 3H), 1.58-1.49 (m, 2H), 1.40 (s, 9H), 1.38-1.22 (m, 1H).

Synthesis of tert-butyl 4-(2-bromoacetyl)azepane-1-carboxylate: Into a 250- mL round-bottom flask purged and maintained with an inert atmosphere of nitrogen, was placed tert-butyl 4-acetylazepane-1-carboxylate (7.3 g, 29.006 mmol, 1.00 equiv), THE (80 mL). This was followed by the addition of LDA (2M in THF, 37 mL, 2.5 equiv) at −78° C. The resulting mixture was stirred at −78° C. for 40 min, treated with TMS-Cl (6.98 mL, 54.6 mmol) over 15 min and stirred for an additional hour. After this time, the reaction mixture was poured into sat NaHCO₃ (100 mL) and extracted with Et₂O (2×100 mL). The extract was washed with brine (2×100 mL), dried (Na₂SO₄) and concentrated on a rotary evaporator. The resultant residue was dissolved in anhydrous THE (150 mL). The resultant solution was cooled to 0° C. and treated sequentially with NaHCO₃ (3.17 g, 37.708 mmol, 1.3 equiv) and NBS (4.65 g, 26.105 mmol, 0.9 equiv). The mixture was stirred at RT for 2 h and then partitioned between saturated NaHCO₃ (100 mL). The mixture was extracted with Et₂O (2×100 mL). The combined ethereal extracts were washed with brine (2×100 mL). The mixture was dried over anhydrous sodium sulfate and concentrated under vacuum. The residue was applied onto a silica gel column with ethyl acetate/petroleum ether (1:3). This resulted in 6 g (Y=64.60%) tert-butyl 4-(2-bromoacetyl) azepane-1-carboxylate as a light yellow oil. LC-MS (ES, m/z): M-56+42: 305. ¹H NMR (300 MHz, DMSO-d₆, ppm) δ 4.48 (s, 2H), 3.47-3.36 (m, 2H), 3.32-3.13 (m, 2H), 2.74 (t, J=10.3 Hz, 1H), 1.99-1.78 (m, 3H), 1.66-1.42 (m, 1H), 1.40 (s, 9H), 1.14-0.99 (m, 1H), 0.97-0.75 (m, 1H)

Synthesis of 1-(azepan-4-yl)-2-bromoethan-1-one hydrochloride: Into a 250- mL round-bottom flask was placed tert-butyl 4-(2-bromoacetyl)azepane-1-carboxylate (6 g, 18.737 mmol, 1.00 equiv), DCM (60 mL), HCl (gas) in 1,4-dioxane (60 mL). The resulting solution was stirred for 1 h at room temperature. The reaction concentrated under vacuum. This resulted in 4.9 g crude 1-(azepan-4-yl)-2-bromoethanone hydrochloride as an off-white solid. LC-MS (ES, m/z): M+1: 220

Synthesis of 1-azabicyclo[3.2.2]nonan-6-one: Into a 1000- mL round-bottom flask was placed K₂CO₃ (7.92 g, 57.294 mmol, 3.0 equiv), ACN (500 mL). This was followed by the addition of 1-(azepan-4-yl)-2-bromoethanone hydrochloride (4.9 g, 19.098 mmol, 1.00 equiv) in ACN (500 mL) at 85 degrees C. The resulting solution was stirred for 3 h at 85 degrees C. The resulting mixture was filtered and the filtrate was concentrated under reduced pressure. The residue was applied onto a silica gel column with dichloromethane/methanol (10:1). This resulted in 2 g (Y=75.23%) 1-azabicyclo [3.2.2]nonan-6-one as a light yellow solid. LC-MS (ES, m/z): M+1: 140. 1H NMR (300 MHz, DMSO-d₆, ppm) δ 3.26-3.19 (m, 2H), 3.08-2.78 (m, 5H), 2.47 (td, J=5.0, 1.9 Hz, 1H), 2.15-1.57 (m, 5H), 1.59-1.38 (m, 1H).

Synthesis of (1R,5S,7R)-7-(hydroxymethyl)-7-(methoxymethyl)-1-azabicyclo[3.2.2]nonan-6-one (racemate) & (1R,5S,7S)-7-(hydroxymethyl)-7-(methoxymethyl)-1-azabicyclo[3.2.2]nonan-6-one (racemate): Into a 100 mL round-bottom flask was placed 1-azabicyclo[3.2.2]nonan-6-one (1.8 g, 12.931 mmol, 1.00 equiv), H₂O (10 mL), MeOH (15 mL), HCHO (30% in H₂O, 12.93 g, 129.310 mmol, 10 equiv), K₂CO₃ (8.94 g, 64.655 mmol, 5 equiv). The resulting solution was stirred for 2 h at 70 degrees C. The resulting mixture was diluted with 50 mL H₂O. The resulting solution was extracted with 2×100 mL of dichloromethane/methanol (10:1) and washed with 2×100 ml of brine. The mixture was dried over anhydrous sodium sulfate and concentrated under vacuum. The crude product was purified by Prep-HPLC with the following conditions: Column, XBridge Shield RP18 OBD Column, Sum, 19*150 mm; mobile phase, Water (0.05% NH₃.H₂O) and ACN (10% Phase B up to 25% in 12 min); Detector, UV 254/220 nm. This resulted in 70 mg (1R,5S,7R)-7-(hydroxymethyl)-7-(methoxymethyl)-1-azabicyclo[3.2.2]nonan-6-one (racemate) as an off-white solid. This resulted in 110 mg (1R,5S,7S)-7-(hydroxymethyl)-7-(methoxymethyl)-1-azabicyclo[3.2.2]nonan-6-one (racemate) as an off-white solid. LC-MS (ES, m/z): M+1: 214. ¹H NMR (400 MHz, Chloroform-d, ppm) δ 4.10 (d, J=11.8 Hz, 1H), 3.82 (dt, J=11.9, 5.7 Hz, 1H), 3.75 (dd, J=9.8, 1.1 Hz, 1H), 3.67 (d, J=9.8 Hz, 1H), 3.41 (d, J=0.8 Hz, 3H), 3.46-3.30 (m, 2H), 3.09-3.01 (m, 2H), 3.02-2.90 (m, 1H), 2.68-2.64 (m, 1H), 2.15-1.97 (m, 1H), 1.91-1.80 (m 2H), 1.83-1.68 (m, 1H), 1.64-1.55 (m, 1H). 1H NMR (400 MHz, Chloroform-d, ppm) δ 3.88 (d, J=10.1 Hz, 1H), 3.80 (d, J=10.3 Hz, 1H), 3.78 (d, J=3.0 Hz, 2H), 3.48-3.40 (m, 1H), 3.39 (s, 3H), 3.39-3.28 (m, 1H), 3.12-3.06 (m 1H), 3.05-2.96 (m, 1H), 2.67-2.63 (m, 1H), 2.21-2.14 (m, 1H), 2.06-1.99 (m, 1H), 1.98-1.90 (m, 2H), 1.78-1.71 (m, 1H), 1.66-1.58 (m, 1H).

Synthesis of (1S,5R,7S)-7-(hydroxymethyl)-7-(methoxymethyl)-1-azabicyclo[3.2.2]nonan-6-one & (1R,5S,7R)-7-(hydroxymethyl)-7-(methoxymethyl)-1-azabicyclo[3.2.2]nonan-6-one: The 70 mg of (1R,5S,7R)-7-(hydroxymethyl)-7-(methoxymethyl)-1-azabicyclo[3.2.2]nonan-6-one (racemate) was purified by Chiral-Prep-HPLC with the following conditions: Column: CHIRALPAK 1H-3, 50*4.6 mm, 3 um 1H30CC-WH004; mobile phase A: n-Hexane (0.2% DEA); mobile phase B: Ethanol:MeOH=1:2; Flow rate: 35 ml/min; Gradient: 10% B to 10% B in 14 min; Detector, 220 nm. This resulted in 3.0 mg of (1S,5R,7S)-7-(hydroxymethyl)-7-(methoxymethyl)-1-azabicyclo[3.2.2]nonan-6-one (Assumed) as an off-white solid. This resulted in 3.2 mg of (1R,5S,7R)-7-(hydroxymethyl)-7-(methoxymethyl)-1-azabicyclo[3.2.2]nonan-6-one (Assumed) as an off-white solid. LC-MS: (ES, m/z): M+1: 214; ee=100%. 1H NMR (400 MHz, Chloroform-d, ppm) δ 4.14 (dd, J=11.8, 2.9 Hz, 1H), 3.84 (dd, J=11.7, 8.5 Hz, 1H), 3.78 (dd, J=9.9, 1.3 Hz, 1H), 3.70 (d, J=9.8 Hz, 1H), 3.44 (s, 3H), 3.48-3.36 (m, 2H), 3.12-3.04 (m, 2H), 2.93 (dd, J=9.1, 3.5 Hz, 1H), 2.70-2.67 (m, 1H), 2.13-2.06 (m, 2H), 1.95-1.83 (m, 2H), 1.80-1.73 (m, 1H), 1.69-1.56 (m, 1H). LC-MS: (ES, m/z): M+1: 214; ee=98%. 1H NMR (400 MHz, Chloroform-d, ppm) δ 4.13 (d, J=11.8 Hz, 1H), 3.89-3.74 (m, 2H), 3.70 (d, J=9.8 Hz, 1H), 3.44 (s, 3H), 3.48-3.35 (m, 2H), 3.14-3.02 (m, 2H), 2.93 (d, J=8.8 Hz, 1H), 2.73-2.64 (m, 1H), 2.15-2.01 (m, 2H), 1.94-1.83 (m, 2H), 1.85-1.69 (m, 1H), 1.69-1.58 (m, 1H).

Synthesis of (1S,5R,7R)-7-(hydroxymethyl)-7-(methoxymethyl)-1-azabicyclo[3.2.2]nonan-6-one & (1R,5S,7S)-7-(hydroxymethyl)-7-(methoxymethyl)-1-azabicyclo[3.2.2]nonan-6-one: The 110 mg of (1R,5S,7S)-7-(hydroxymethyl)-7-(methoxymethyl)-1-azabicyclo[3.2.2]nonan-6-one (racemate) was purified by Anal-SFC with the following conditions: Column, Lux-2 100*4.6.0 mm 3.0 um; mobile phase: IPA (50% Hex); Flow rate: 35 ml/min; Gradient: 0% B to 10% B in 14 min; Detector, 220 nm. This resulted in 3.8 mg of (1S,5R,7R)-7-(hydroxymethyl)-7-(methoxymethyl)-1-azabicyclo[3.2.2]nonan-6-one (Assumed) as a light yellow oil. This resulted in 3.5 mg of (1R,5S,7S)-7-(hydroxymethyl)-7-(methoxymethyl)-1-azabicyclo[3.2.2]nonan-6-one (Assumed) as a light yellow oil. LC-MS: (ES, m/z): M+1: 214; ee=100%. ¹H NMR (300 MHz, Chloroform-d) δ 3.89 (d, J=10.2 Hz, 1H), 3.85-3.74 (m, 3H), 3.50-3.41 (m, 1H), 3.39 (s, 3H), 3.38-3.30 (m, 1H), 3.23 (s, 1H), 3.17-2.97 (m, 2H), 2.72-2.62 (m, 1H), 2.22-2.15 (m, 1H), 2.07-1.88 (m, 1H), 1.81-1.71 (m, 1H), 1.74-1.59 (m, 1H). LC-MS: (ES, m/z): M+1: 214; ee=98%. 1H NMR (300 MHz, Chloroform-d) δ 3.89 (d, J=10.2 Hz, 1H), 3.80 (d, J=12.8 Hz, 3H), 3.45 (dd, J=14.9, 7.2 Hz, 1H), 3.39 (s, 3H), 3.37-3.30 (m, 1H), 3.14-2.99 (m, 2H), 2.67-2.64 (m, 1H), 2.22-2.14 (m, 1H), 2.09-1.86 (m, 3H), 1.7-1.71 (m, 1H), 1.67-1.59 (m, 1H).

### Example 21: Preparation of bis((2-(methoxymethyl)-3-oxoquinuclidin-2-yl)methyl) methylphosphonate

Synthesis of 2-(hydroxymethyl)-2-(methoxymethyl)-1-azabicyclo[2.2.2]octan-3-one: Into a 1000-mL round-bottom flask, was placed quinuclidin-3-one (50.00 g, 399.45 mmol). This was followed by the addition of K₂CO₃ (55.21 g, 399.45 mmol, 1.00 equiv), H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. The mixture was dried over anhydrous sodium sulfate and concentrated. The residue was applied onto a silica gel column with ethyl acetate/petroleum ether (0:1-1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of bis((2-(methoxymethyl)-3-oxoquinuclidin-2-yl)methyl) methylphosphonate: To a stirred solution of 2-(hydroxymethyl)-2-(methoxymethyl)-1-azabicyclo[2.2.2]octan-3-one (150 mg, 0.753 mmol, 1.0 equiv) and TEA (150.2 mg, 1.506 mmol, 2.00 equiv) in DCM was added methylphosphonic dichloride (60.0 mg, 0.452 mmol, 0.6 equiv) at 0° C. The resulting mixture was stirred for 4 h at 0° C. The resulting mixture was extracted with CH₂Cl₂ (2×10 mL). The combined organic layers were washed with brine (1×10 mL), dried over anhydrous Na₂SO₄. After filtration, the filtrate was concentrated under reduced pressure. The crude product was purified by Prep-HPLC with the following conditions (Column: Kinetex EVO C18 Column, 21.2*150, 5 um; Mobile Phase A: Water (0.05% NH₃H₂O), Mobile Phase B: ACN; Flow rate: 20 mL/min; Gradient: 13% B to 26% B in 8 min, 26% B; Wave Length: 220 NM nm; RT1 (min): 7.6;) to afford bis((2-(methoxymethyl)-3-oxoquinuclidin-2-yl)methyl) methylphosphonate (30 mg, 8.69%) as a white solid. LC-MS: (ES, m/z): M+1: 459. ¹H NMR (400 MHz, DMSO-d₆) δ 4.75-4.61 (m, 1H), 4.47 (dd, J=11.9, 5.7 Hz, 2H), 3.61 (t, J=12.0 Hz, 5H), 3.53-3.39 (m, 5H), 3.32 (s, 6H), 2.76-2.65 (m, 3H), 2.19 (dd, J=39.8, 12.1 Hz, 11H), 1.68 (d, J=18.1 Hz, 3H).

### Example 22: Preparation of tris((2-(methoxymethyl)-3-oxoquinuclidin-2-yl)methyl) phosphate hydrochloride

Synthesis of 2-(hydroxymethyl)-2-(methoxymethyl)-1-azabicyclo[2.2.2]octan-3-one: Into a 1000-mL round-bottom flask, was placed quinuclidin-3-one (50.00 g, 399.45 mmol). This was followed by the addition of K₂CO₃ (55.21 g, 399.45 mmol, 1.00 equiv), H₂O (200.00 mL), MeOH (300.00 mL). The resulting solution was stirred for 5 h at 75 degrees C. Then the resulting solution was stirred for overnight at room temperature. The resulting solution was extracted with 3×500 mL of dichloromethane concentrated. The mixture was dried over anhydrous sodium sulfate and concentrated. The residue was applied onto a silica gel column with ethyl acetate/petroleum ether (0:1-1:1). This resulted in 11 g (13.82%) of 2-(hydroxymethyl)-2-(methoxymethyl)-1-azabicyclo[2.2.2]octan-3-one as a white solid. LC-MS: (ES, m/z): M+1: 200. ¹H NMR (CDCl₃, 300 ppm) 3.98 (d, J=11.7 Hz, 1H), 3.83-3.79 (m, 3H), 3.39-3.28 (m, 5H), 3.02-2.89 (m, 2H), 2.43-2.38 (m, 1H), 2.11-2.02 (m, 2H).

Synthesis of Synthesis of tris((2-(methoxymethyl)-3-oxoquinuclidin-2-yl)methyl) phosphate hydrochloride: Into a 250-mL round-bottom flask purged and maintained with an inert atmosphere of nitrogen, was placed 2-(hydroxymethyl)-2-(methoxymethyl)-1-azabicyclo[2.2.2]octan-3-one (389.84 mg, 1.956 mmol, 3 equiv), DCM (10 mL), TEA (263.98 mg, 2.608 mmol, 4 equiv). This was followed by the addition of POCl₃ (100 mg, 0.652 mmol, 1.00 equiv) at 0 degrees C. The resulting solution was stirred for 16 h at room temperature. The reaction was then quenched by the addition of 20 mL of water. The resulting solution was extracted with 2×20 mL of dichloromethane/methanol (10:1) and washed with 2×30 ml of brine. The mixture was dried over anhydrous sodium sulfate and concentrated under vacuum. The crude product was purified by Prep-HPLC with the following conditions: SunFire Prep C18 OBD Column, 50*250 mm Sum 10 nm; mobile phase, phase A: H₂O (0.05% TFA); phase B: CH₃CN (10% CH₃CN up to 40% CH₃CN in 12 min). This resulted in 30 mg (7.17%) of tris((2-(methoxymethyl)-3-oxoquinuclidin-2-yl)methyl) phosphate hydrochloride as a white solid. LC-MS: (ES, m/z): M+1: 678. ¹H NMR (400 MHz, DMSO-d₆) δ 11.84 (s, 2H), 4.96 (dd, J=12.0, 4.1 Hz, 3H), 4.58 (dt, J=12.7, 3.9 Hz, 3H), 4.10-3.97 (m, 6H), 3.79-3.57 (m, 6H), 3.47 (s, 6H), 3.39-3.25 (m, 9H), 2.70 (s, 3H), 2.27-2.14 (m, 12H).

### Example 23: Preparation of (1S,2S,4R,6S)-2-(hydroxymethyl)-2-(methoxymethyl)-6-(trifluoromethyl)quinuclidin-3-one (Assumed) and (1R,2R,4S,6R)-2-(hydroxymethyl)-2-(methoxymethyl)-6-(trifluoromethyl)quinuclidin-3-one (Assumed) and (1S,2R,4R,6S)-2-(hydroxymethyl)-2-(methoxymethyl)-6-(trifluoromethyl)quinuclidin-3-one (Assumed) and (1R,2S,4S,6R)-2-(hydroxymethyl)-2-(methoxymethyl)-6-(trifluoromethyl)quinuclidin-3-one (Assumed)

Synthesis of 2-(trifluoromethyl)pyridine-4-carboxylate: Into a 1000 mL pressure tank reactor were added 4-bromo-2-(trifluoromethyl)pyridine (50 g, 221.243 mmol, 1.00 equiv), MeOH (500 mL), TEA (44.78 g, 442.486 mmol, 2 equiv), Pd(dppf)Cl₂CH₂Cl₂ (5.41 g, 6.637 mmol, 0.03 equiv) at room temperature. The resulting mixture was stirred for 16 h at 80 degrees C. under 20 atm carbon monoxide atmosphere. The mixture was allowed to cool down to room temperature. The resulting mixture was filtered, the filter cake was washed with MeOH (200 mL). The filtrate was concentrated under reduced pressure. The resulting mixture was filtered, the filter cake was washed with MeOH (1×100 mL). The filtrate was concentrated under reduced pressure. This resulted in methyl 2-(trifluoromethyl)pyridine-4-carboxylate (42 g, 92.54%) as colorless oil. LC-MS: (ES, m/z): M+1=206.

Synthesis of methyl 2-tert-butylpiperidine-4-carboxylate: Into a 1000 mL pressure tank reactor were added methoxy[2-(trifluoromethyl)pyridin-4-yl]methanol (42 g, 202.750 mmol, 1.00 equiv), MeOH (300 mL), HOAc (100 mL), PtO₂ (4.60 g, 20.275 mmol, 0.1 equiv) at room temperature. The resulting mixture was stirred for 60 h at 100 degrees C. under 60 atm hydrogen atmosphere. The mixture was allowed to cool down to room temperature. The resulting mixture was filtered, the filter cake was washed with MeOH (1×150 mL). The filtrate was concentrated under reduced pressure. residue was purified by silica gel column chromatography, eluted with PE/EA (10:01-1:1) to afford methyl 2-tert-butylpiperidine-4-carboxylate (25.6 g, 63.36%) as colorless oil. LC-MS: (ES, m/z): M+1=212. ¹H NMR (300 MHz, DMSO-d, ppm) δ 3.62 (s, 3H), 3.34-3.12 (m, 1H), 3.08-2.94 (m, 1H), 2.56 (dt, J=12.4, 3.4 Hz, 1H), 1.97-1.93 (m, 1H), 1.79 (ddq, J=12.9, 4.4, 2.4 Hz, 1H), 1.47-1.26 (m, 2H).

Synthesis of methyl 1-(2-methoxy-2-oxoethyl)-2-(trifluoromethyl)piperidine-4-carboxylate: Into a 1000 mL 3-necked round-bottom flask were added methyl 2-(trifluoromethyl)piperidine-4-carboxylate (22 g, 104.175 mmol, 1.00 equiv) DMF (220 mL), methyl 2-bromoacetate (23.90 g, 156.262 mmol, 1.5 equiv), Cs₂CO₃ (67.88 g, 208.350 mmol, 2 equiv) at room temperature. The resulting mixture was stirred for 12 h at 110 degrees C. under nitrogen atmosphere. The mixture was allowed to cool down to room temperature. The resulting mixture was diluted with water (300 mL). The resulting mixture was extracted with EtOEt (3×200 mL). The combined organic layers were washed with brine (1×200 mL), dried over anhydrous Na₂SO₄. After filtration, the filtrate was concentrated under reduced pressure. The residue was purified by silica gel column chromatography, eluted with PE/EA (10:01-3:1) to afford methyl 1-(2-methoxy-2-oxoethyl)-2-(trifluoromethyl)piperidine-4-carboxylate (23 g, 77.95%) as colorless oil. LC-MS: (ES, m/z): M+1=284. ¹H NMR (300 MHz, DMSO-d, ppm) δ 4.72 (s, 2H), 3.76 (s, 3H), 3.70 (s, 3H), 3.04-2.85 (m, 2H), 2.62-2.51 (m, J=4.8, 1.3 Hz, 1H), 2.17-2.10 (m, 2H), 1.89-1.81 (m, 1H), 1.60-1.37 (m, 2H).

Synthesis of methyl 3-oxo-6-(trifluoromethyl)-1-azabicyclo[2.2.2]octane-2-carboxylate: Into a 1000 mL 3-necked round-bottom flask were added methyl 1-(2-methoxy-2-oxoethyl)-2-(trifluoromethyl)piperidine-4-carboxylate (18 g, 63.549 mmol, 1.00 equiv), Toluene (500 mL), t-BuOK (21.39 g, 190.647 mmol, 3 equiv) at room temperature. The resulting mixture was stirred for 2 h at d110 degrees C. The mixture was allowed to cool down to room temperature. The resulting mixture was diluted with water (300 mL). The resulting mixture was extracted with EtOEt (3×200 mL). The combined organic layers were washed with brine (lx 200 mL), dried over anhydrous Na₂SO₄. After filtration, the filtrate was concentrated under reduced pressure. The residue was purified by silica gel column chromatography, eluted with PE/EA (10:01-3:1) to afford methyl 3-oxo-6-(trifluoromethyl)-1-azabicyclo[2.2.2]octane-2-carboxylate (4.5 g, 28.19%) as colorless oil. LC-MS: (ES, m/z): M+1=252

Synthesis of 6-(trifluoromethyl)-1-azabicyclo[2.2.2]octan-3-one: Into a 100 mL round-bottom flask were added methyl 2-methyl-3-oxo-3-[2-(trifluoromethyl)piperidin-4-yl]propanoate (4.5 g, 16.838 mmol, 1.00 equiv) and 6N HCl (45 mL, at room temperature. The resulting mixture was stirred for 12 h at 100 degrees C. The mixture was allowed to cool down to room temperature. The resulting mixture was diluted with water (200 mL). The resulting mixture was extracted with EtOEt (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (10:01-3:1) to afford 6-(trifluoromethyl)-1-azabicyclo[2.2.2]octan-3-one (1.5 g, 46.12%) as colorless oil. LC-MS: (ES, m/z): M+1=194. ¹H NMR (300 MHz, DMSO-d, ppm) δ 3.77 (p, J=9.5 Hz, 1H), 3.39-3.28 (m, J=5.1 Hz, 2H), 3.25-2.99 (m, 1H), 2.89-2.66 (m, 1H), 2.39 (d, J=3.7 Hz, 1H), 2.5-2.10 (m, J=13.6, 10.2, 3.4 Hz, 1H), 2.05-1.72 (m, 3H).

Synthesis of 3-hydroxy-2-(methoxymethyl)-2-methyl-1-[2-(trifluoromethyl)piperidin-4-yl]propan-1-one: Into a 100 mL 3-necked round-bottom flask were added 6-(trifluoromethyl)-1-azabicyclo[2.2.2]octan-3-one (1.5 g, 7.765 mmol, 1.00 equiv), MeOH (12 mL), H₂O (10 mL), 30% HCHO (2.33 g, 77.650 mmol, 10 equiv), K₂CO₃ (4.29 g, 31.060 mmol, 4 equiv) at room temperature. The resulting mixture was stirred for 2 h at 70 degrees C. The mixture was allowed to cool down to room temperature. The resulting mixture was diluted with water (20 mL). The resulting mixture was extracted with EtOEt (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EA (10:01-3:1) to afford 3-hydroxy-2-(methoxymethyl)-2-methyl-1-[2-(trifluoromethyl)piperidin-4-yl]propan-1-one (racemate) (180 mg, 8.18%) as white solid. The crude product was purified by Prep-HPLC with the following conditions (Prep-HPLC-003): Column, SunFire Prep C18 OBD Column, 19*150 mm, 5 μm 10 nm; mobile phase, Water (0.05% TFA) and ACN (35% ACN up to 75% in 10 min); Detector, uv. The collected solution was concentrated under vacuum to remove CH₃CN and the resulting solution was dried by lyophilization. Peak Q: M+1=268, R,T=0.723 min, This resulted in (80 mg, 44.4%) as white solid. Peak H: M+1=268, R,T=0.801 min, This resulted in (85 mg, 47.2%) as white solid.

### Synthesis of (1S,2S,4R,6S)-2-(hydroxymethyl)-2-(methoxymethyl)-6-(trifluoromethyl)quinuclidin-3-one (Assumed) and (1R,2R,4S,6R)-2-(hydroxymethyl)-2-(methoxymethyl)-6-(trifluoromethyl)quinuclidin-3-one (Assumed)

The Peak H (85 mg) was purified by Chiral-Prep-HPLC with the following conditions: Mobile phase: A: n-Hexane (0.1%) B: MeOH; Flow rate: 20 mL/min; Column: DAICEL CHIRALPAK IA, 250*20 mm, Sum; Gradient: 12% B in 20 min; 220 nm. This resulted in 30 mg (35.29%) of (1S,2S,4R,6S)-2-(hydroxymethyl)-2-(methoxymethyl)-6-(trifluoromethyl)quinuclidin-3-one (Assumed) as off-white solid. And 25 mg (29.41%) of (1R,2R,4S,6R)-2-(hydroxymethyl)-2-(methoxymethyl)-6-(trifluoromethyl)quinuclidin-3-one (Assumed) as off-white solid.

(1S,2S,4R,6S)-2-(hydroxymethyl)-2-(methoxymethyl)-6-(trifluoromethyl)quinuclidin-3-one (Assumed): LC-MS-A: (ES, m/z): M+1=268. 1H NMR-A (300 MHz, DMSO-d, ppm) δ 4.19 (p, J=8.9 Hz, 1H), 3.95 (d, J=10.8 Hz, 1H), 3.91-3.82 (m, 3H), 3.36 (s, 3H), 3.28 (td, J=10.4, 9.9, 5.1 Hz, 1H), 3.12 (t, J=12.8 Hz, 1H), 2.53 (s, 2H), 2.32 (dd, J=13.4, 10.4 Hz, 1H), 2.17-2.02 (m, 2H), 1.96 (s, 1H).

(1R,2R,4S,6R)-2-(hydroxymethyl)-2-(methoxymethyl)-6-(trifluoromethyl)quinuclidin-3-one (Assumed): LC-MS-D: (ES, m/z): M+1=268. 1H NMR- -D (300 MHz, DMSO-d, ppm) δ 4.32 (q, J=9.0 Hz, 1H), 3.96 (s, 2H), 3.93 (s, 2H), 3.37 (s, 3H), 3.33-3.17 (m, 2H), 2.65-2.54 (s, 2H), 2.41-2.28 (m, 1H), 2.21-2.03 (m, 2H), 2.05-1.88 (m, 1H).

Synthesis of (1S,2R,4R,6S)-2-(hydroxymethyl)-2-(methoxymethyl)-6-(trifluoromethyl)quinuclidin-3-one (Assumed) and (1R,2S,4S,6R)-2-(hydroxymethyl)-2-(methoxymethyl)-6-(trifluoromethyl)quinuclidin-3-one (Assumed): The Peak Q (80 mg) was separated by SFC with the following conditions: Column, CHIRALPAK AY-3, 50*4.6 mm, 3 um AY30CC-SK001; mobile phase A: n-Hexane (0.1% DEA); mobile phase B: Ethanol; Flow rate: 1.0 ml/min; Gradient: 20% B (5 min, RT: 1.675 min, 1.908 min); Detector, 220 nm. This resulted in (1S,2R,4R,6S)-2-(hydroxymethyl)-2-(methoxymethyl)-6-(trifluoromethyl)quinuclidin-3-one (Assumed) (25 mg, 31.25%) as colorless oil. And (1R,2S,4S,6R)-2-(hydroxymethyl)-2-(methoxymethyl)-6-(trifluoromethyl)quinuclidin-3-one (Assumed) (20 mg, 25.00%) as colorless oil.

(1S,2R,4R,6S)-2-(hydroxymethyl)-2-(methoxymethyl)-6-(trifluoromethyl)quinuclidin-3-one (Assumed): LC-MS-B: (ES, m/z): M+1=268. ¹H NMR-B (300 MHz, DMSO-d, ppm) δ 4.08-3.87 (m, 3H), 3.86 (d, J=10.0 Hz, 1H), 3.74 (d, J=10.4 Hz, 1H), 3.41 (s, 3H), 3.34 (t, J=8.1 Hz, 1H), 3.20 (dt, J 15.6, 7.4 Hz, 1H), 2.66 (s, 1H), 2.53 (s, 1H), 2.34-2.09 (i, 2H), 2.04 (s, 2H).

(1R,2S,4S,6R)-2-(hydroxymethyl)-2-(methoxymethyl)-6-(trifluoromethyl)quinuclidin-3-one (Assumed): LC-MS-C: (ES, m/z): M+1=268. ¹H NMR-C (300 MHz, DMSO-d, ppm) δ 4.08-3.84 (m, 3H), 3.85 (d, J=6.1 Hz, 1H), 3.73 (d, J=10.4 Hz, 1H), 3.40 (s, 3H), 3.38-3.27 (m, 1H), 3.20 (dt, J=15.1, 7.4 Hz, 1H), 2.52 (s, 1H), 2.21 (dq, J=20.7, 13.5 Hz, 2H), 2.05 (d, J=8.5 Hz, 2H), 1.27 (s, 1H).

Example A: The compounds below are prepared by methods substantially identical, similar, or analogous to those disclosed in the General Scheme and above Examples.

| Cmpound | Chemical Structure | m/z[MH + 1] |
|---|---|---|
| 1. | (1R,2R,4S,6R)-2-(hydroxymethyl)-6-methyl-2- | 268 |
| | ((trifluoromethoxy)methyl)quinuclidin-3-one | |
| 2. | (1R,2R,4S,6R)-2-(hydroxymethyl)-2-((methoxy- | 217 |
| | d3)methyl)-6-methylquinuclidin-3-one, | |
| 3. | (1R,2R,4S,6R)-6-ethyl-2-(hydroxymethyl)-2- | 228 |
| | (methoxymethyl)quinuclidin-3-one, | |
| 4. | (1S,2R,4R,6R)-6-ethyl-2-(hydroxymethyl)-2- | 228 |
| | (methoxymethyl)quinuclidin-3-one, | |
| 5. | (1R,2S,4S,6R)-6-ethyl-2-(hydroxymethyl)-2- | 228 |
| | (methoxymethyl)quinuclidin-3-one, | |
| 6. | (1S,2R,4R,6S)-6-ethyl-2-(hydroxymethyl)-2- | 228 |
| | (methoxymethyl)quinuclidin-3-one, | |
| 7. | (1R,2R,4S,6S)-6-ethyl-2-(hydroxymethyl)-2- | 228 |
| | (methoxymethyl)quinuclidin-3-one, | |
| 8. | (1S,2S,4R,6R)-6-ethyl-2-(hydroxymethyl)-2- | 228 |
| | (methoxymethyl)quinuclidin-3-one, | |
| 9. | (1S,2S,4R,6S)-6-ethyl-2-(hydroxymethyl)-2- | 228 |
| | (methoxymethyl)quinuclidin-3-one, | |
| 10. | (1R,2S,4S,6S)-6-ethyl-2-(hydroxymethyl)-2- | 228 |
| | (methoxymethyl)quinuclidin-3-one, | |
| 11. | (1R,2S,4S,6R)-2-(hydroxymethyl)-6-isopropyl-2- | 242 |
| | (methoxymethyl)quinuclidin-3-one, | |
| 12. | (1S,2R,4R,6S)-2-(hydroxymethyl)-6-isopropyl-2- | 242 |
| | (methoxymethyl)quinuclidin-3-one, | |
| 13. | (1R,2R,4S,6S)-2-(hydroxymethyl)-6-isopropyl-2- | 242 |
| | (methoxymethyl)quinuclidin-3-one, | |
| 14. | (1S,2S,4R,6S)-2-(hydroxymethyl)-6-isopropyl-2- | 242 |
| | (methoxymethyl)quinuclidin-3-one, | |
| 15. | (1R,2S,4S,6S)-2-(hydroxymethyl)-6-isopropyl-2- | 242 |
| | (methoxymethyl)quinuclidin-3-one, | |
| 16. | (1S,2S,4R,6R)-2-(hydroxymethyl)-6-isopropyl-2- | 242 |
| | (methoxymethyl)quinuclidin-3-one, | |
| 17. | (1R,2R,4S,6R)-2-(hydroxymethyl)-6-isopropyl-2- | 242 |
| | (methoxymethyl)quinuclidin-3-one, | |
| 18. | (1S,2R,4R,6R)-2-(hydroxymethyl)-6-isopropyl-2- | 242 |
| | (methoxymethyl)quinuclidin-3-one, | |
| 19. | (1S,2R,4R,6S)-2-(hydroxymethyl)-6-isobutyl-2- | 256 |
| | (methoxymethyl)quinuclidin-3-one, | |
| 20. | (1S,2S,4R,6S)-2-(hydroxymethyl)-6-isobutyl-2- | 256 |
| | (methoxymethyl)quinuclidin-3-one, | |
| 21. | (1R,2R,4S,6R)-2-(hydroxymethyl)-6-isobutyl-2- | 256 |
| | (methoxymethyl)quinuclidin-3-one, | |
| 22. | (1R,2S,4S,6R)-2-(hydroxymethyl)-6-isobutyl-2- | 256 |
| | (methoxymethyl)quinuclidin-3-one, | |
| 23. | (1S,2R,4R,6R)-2-(hydroxymethyl)-6-isobutyl-2- | 256 |
| | (methoxymethyl)quinuclidin-3-one, | |
| 24. | (1S,2S,4R,6R)-2-(hydroxymethyl)-6-isobutyl-2- | 256 |
| | (methoxymethyl)quinuclidin-3-one, | |
| 25. | (1R,2R,4S,6S)-2-(hydroxymethyl)-6-isobutyl-2- | 256 |
| | (methoxymethyl)quinuclidin-3-one, | |
| 26. | (1R,2S,4S,6S)-2-(hydroxymethyl)-6-isobutyl-2- | 256 |
| | (methoxymethyl)quinuclidin-3-one | |
| 27. | (1S,2R,4R,6R)-2-(hydroxymethyl)-2-(methoxymethyl)-6- | 270 |
| | neopentylquinuclidin-3-one, | |
| 28. | (1S,2S,4R,6R)-2-(hydroxymethyl)-2-(methoxymethyl)-6- | 270 |
| | neopentylquinuclidin-3-one, | |
| 29. | (1R,2R,4S,6S)-2-(hydroxymethyl)-2-(methoxymethyl)-6- | 270 |
| | neopentylquinuclidin-3-one, | |
| 30. | (1R,2S,4S,6S)-2-(hydroxymethyl)-2-(methoxymethyl)-6- | 270 |
| | neopentylquinuclidin-3-one, | |
| 31. | (1S,2R,4R,6S)-2-(hydroxymethyl)-2-(methoxymethyl)-6- | 270 |
| | neopentylquinuclidin-3-one | |
| 32. | (1S,2R,4R,6S)-2-(hydroxymethyl)-2-(methoxymethyl)-6- | 270 |
| | neopentylquinuclidin-3-one, | |
| 33. | (1R,2R,4S,6R)-2-(hydroxymethyl)-2-(methoxymethyl)-6- | 270 |
| | neopentylquinuclidin-3-one, | |
| 34. | (1R,2S,4S,6R)-2-(hydroxymethyl)-2-(methoxymethyl)-6- | 270 |
| | neopentylquinuclidin-3-one, | |
| 35. | (1S,2R,4R)-2-(hydroxymethyl)-2-(methoxymethyl)-1- | 186 |
| | azabicyclo[2.2.1]heptan-3-one, | |
| 36. | (1S,2S,4R)-2-(hydroxymethyl)-2-(methoxymethyl)-1- | 186 |
| | azabicyclo[2.2.1]heptan-3-one, | |
| 37. | (1R,2R,4S)-2-(hydroxymethyl)-2-(methoxymethyl)-1- | 186 |
| | azabicyclo[2.2.1]heptan-3-one, | |
| 38. | (1R,2S,4S)-2-(hydroxymethyl)-2-(methoxymethyl)-1- | 186 |
| | azabicyclo[2.2.1]heptan-3-one, | |
| 39. | isopropyl (isopropoxy(((1R,2S,4S,6R)-2-(methoxymethyl)- | 525 |
| | 6-methyl-3-oxoquinuclidin-2-yl)methoxy)phosphoryl)-L- | |
| | phenylalaninate, | |
| 40. | isopropyl ((((1R,2S,4S,6R)-2-(methoxymethyl)-6-methyl-3- | 678 |
| | oxoquinuclidin-2-yl)methoxy)(((2S,6R)-2- | |
| | (methoxymethyl)-6-methyl-3-oxoquinuclidin-2- | |
| | yl)methoxy)phosphoryl)-L-phenylalaninate, | |
| 41. | ((1R,2S,4S,6R)-2-(methoxymethyl)-6-methyl-3- | 565 |
| | oxoquinuclidin-2-yl)methyl(((2S,6R)-2-(methoxymethyl)- | |
| | 6-methyl-3-oxoquinuclidin-2-yl)methyl)phenyl phosphate, | |
| 42. | ((1R,2S,4S,6R)-2-(methoxymethyl)-6-methyl-3- | 487 |
| | oxoquinuclidin-2-yl)methyl(((2S,6R)-2-(methoxymethyl)- | |
| | 6-methyl-3-oxoquinuclidin-2-yl)methyl)methylphosphonate, | |
| 43. | isopropyl ((((1R,2S,4S,6R)-2-(methoxymethyl)-6-methyl-3- | 559 |
| | oxoquinuclidin-2-yl)methoxy)(phenoxy)phosphoryl)-L- | |
| | phenylalaninate, | |
| 44. | benzyl ((((1R,2S,4S,6R)-2-(methoxymethyl)-6-methyl-3- | 559 |
| | oxoquinuclidin-2-yl)methoxy)(phenoxy)phosphoryl)-L- | |
| | valinate, | |
| 45. | ((1R,2S,4S,6R)-2-(methoxymethyl)-6-methyl-3- | 256 |
| | oxoquinuclidin-2-yl)methyl acetate, | |
| 46. | ((1R,2S,4S,6R)-2-(methoxymethyl)-6-methyl-3- | 284 |
| | oxoquinuclidin-2-yl)methyl isobutyrate, | |
| 47. | ((1R,2S,4S,6R)-2-(methoxymethyl)-6-methyl-3- | 298 |
| | oxoquinuclidin-2-yl)methyl pivalate, | |
| 48. | benzyl ((((1R,2S,4S,6R)-2-(methoxymethyl)-6-methyl-3- | 531 |
| | oxoquinuclidin-2-yl)methoxy)(phenoxy)phosphoryl)-L- | |
| | alaninate, | |
| 49. | isopropyl ((((1R,2S,4S,6R)-2-(methoxymethyl)-6-methyl-3- | 511 |
| | oxoquinuclidin-2-yl)methoxy)(phenoxy)phosphoryl)-L- | |
| | valinate, | |
| 50. | isopropyl ((((1R,2S,4S,6R)-2-(methoxymethyl)-6-methyl-3- | 483 |
| | oxoquinuclidin-2-yl)methoxy)(phenoxy)phosphoryl)-L- | |
| | alaninate, | |
| 51. | isopropyl ((((1R,2S,4S,6R)-2-(methoxymethyl)-6-methyl-3- | 611 |
| | oxoquinuclidin-2-yl)methoxy)((S)-2-methyl-1- | |
| | (propionyloxy)propoxy)phosphoryl)-L-phenylalaninate, | |
| 52. | benzyl (isopropoxy(((1R,2S,4S,6R)-2-(methoxymethyl)-6- | 525 |
| | methyl-3-oxoquinuclidin-2-yl)methoxy)phosphoryl)-L- | |
| | valinate, | |

### Biological Example 1: In Vitro Anti-Proliferation Assay in BCL-2-Dependent Acute Lymphoblastic Leukemia (ALL) Cell Line RS4;11 with G101V Mutation

Certain Bcl-2 inhibitors such as venetoclax induce high rates of durable remission in patients with previously treated leukemia such as chronic lymphocytic leukemia (CLL). However, disease can relapse in certain patients. In some replacing patients, a recurring secondary mutation is the Gly101Val (G101V) mutation, which reduces the affinity of venetoclax to Bcl-2 by ˜180-fold in surface plasmon resonance assays, thereby preventing the drug from displacing pro-apoptotic mediators from Bcl-2 in cells, and conferring acquired resistance in cell lines and primary patient cells.

The RS4;11 cell line used herein was engineered to stably overexpress the G101V mutant form of human Bcl-2. The cell antiproliferation was assayed by PerkinElmer ATPlite™ Luminescence Assay System. Briefly, the RS4;11(G101V) cancer cells were plated at a density of about 1×10⁴ cells per well in Costar 96-well plates, and were incubated with different concentrations of compounds for about 72 hours in medium supplemented with 5% FBS or 10% normal human serum (NHS). One lyophilized substrate solution vial was then reconstituted by adding 5 mL of substrate buffer solution, and was agitated gently until the solution was homogeneous. About 50 μL of mammalian cell lysis solution was added to 100 μL of cell suspension per well of a microplate, and the plate was shaken for about five minutes in an orbital shaker at ˜700 rpm. This procedure was used to lyse the cells and to stabilize the ATP. Next, 50 μL substrate solution was added to the wells and microplate was shaken for five minutes in an orbital shaker at ˜700 rpm. Finally, the luminescence was measured by a PerkinElmer TopCount® Microplate Scintillation Counter. Such assays, carried out with a range of doses of test compounds, allowed the determination of the cellular anti-antiproliferative IC₅₀ of the compounds of the present invention. The following table lists the IC₅₀ values of certain compounds of the invention.

The following table lists the IC₅₀ values of certain compounds of the invention.

| | RS4; 11-G101V |
|---|---|
| Compound | IC50 (μM) |
| APR-246 | <5 |
| Example 2 | <5 |
| Example 3 (isomer 1) | <5 |
| Example 3 (isomer 2) | >5 |
| Example 4 | >5 |
| Example 5 | <5 |
| Example 6(isomer 1) | <5 |
| Example 6(isomer 2) | <5 |
| Example 7(isomer 1) | <5 |
| Example 7(isomer 2) | <5 |
| Example 8(isomer 1) | <5 |
| Example 8(isomer 2) | <5 |
| Example 9 | >5 |
| Example 10 | >5 |
| Example 15 | <5 |
| Example 16 | <5 |

The following table lists the IC₅₀ values of another in vitro anti-proliferation assay in RS4;11-G101V cell line. The Example 15 is more potent than the reference compound APR-246.

| Compound | IC50 (uM) |
|---|---|
| APR-246 | 1.76 |
| Example 15 | 0.73 |

### Biological Example 2: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing was 5% DMSO in 20% HPBCD in water, and the PO formulation was 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation.

The PK results of Example 15 is shown in the Table below. The data shows that Example has excellent bioavailability.

| Example 15 | AUCₗₐₛ(h*ng/mL) | Bioavailabilty |
|---|---|---|
| Oral, 100 mpk | 26,093 | 79% |

### Biological Example 3: In Vivo Xenograft Studies

Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

What is claimed is:

1. A compound of Formula (I), or an N-oxide thereof, or a pharmaceutically acceptable salt, solvate, polymorph, tautomer, stereoisomer, an isotopic form, or a prodrug of said compound of Formula (I) or N-oxide thereof: 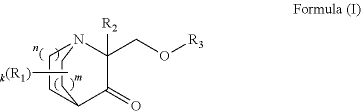
wherein
k is 1, 2, 3, 4, 5, or 6;
R₁ is alkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, alkenyl, alkynyl, cycloalkyl, cycloalkenyl, heterocycloalkyl, spiroheterocycloalkyl, heterocycloalkenyl, aryl, heteroaryl is optionally substituted with one or more R_{d};
R₂ is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, alkyl-ORₐ, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, —S(O)(═N(R_{b}))R_{c}, —N═S(O)R_{b}R_{c}, ═NR_{b}, —SO₂N(R_{b})R_{c}, —N(R_{b})SO₂R_{c}, in which said cycloalkyl, cycloalkenyl, heterocycloalkyl, heterocycloalkenyl, aryl, heteroaryl is optionally substituted with one or more R_{d};
R₃ is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, —S(O)(═N(R_{b}))R_{c}, —N═S(O)R_{b}R_{c}, ═NR_{b}, —SO₂N(R_{b})R_{c}, —N(R_{b})SO₂R_{c}, 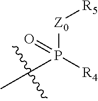
in which said cycloalkyl, cycloalkenyl, heterocycloalkyl, heterocycloalkenyl, aryl, heteroaryl is optionally substituted with one or more R_{d};
Z₀ is absent, O, N(Rₐ), or S;
R₄ is alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH—(CHR_{b})COOR_{c}, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
R₅ is alkyl, spiroalkyl, alkenyl, alkynyl, cycloalkyl, cycloalkenyl, heterocycloalkyl, spiroheterocycloalkyl, heterocycloalkenyl, aryl, heteroaryl, -alkyl-Rₐ, —NH(CH₂)ₚRₐ, —C(O)Rₐ, —S(O)Rₐ, —SO₂Rₐ, —C(O)ORₐ, —C(O)ORₐ, -alkyl-OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, C(O)NHOH, C(O)OH, —C(O)O-alkyl, —C(O)O-aryl, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ; and
Rₑ is H, D, alkyl, spiroalkyl, alkenyl, alkynyl, halo, cyano, amine, nitro, hydroxy, ═O, C(O)NHOH, —C(O)O-alkyl, —C(O)O-aryl, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl.
two of R₁ groups, taken together with the atom to which they are attached, may optionally form a cycloalkyl or heterocycloalkyl optionally substituted with one or more R_{d};
R₂ and R₃ groups, taken together with the atom to which they are attached, may optionally form a cycloalkyl or heterocycloalkyl optionally substituted with one or more R_{d}; and
each of m, n, k, and p, independently, is 0, 1, 2, or 3.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (II):

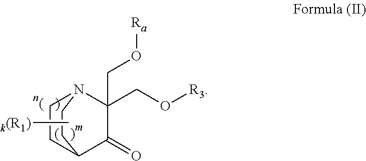

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (III):

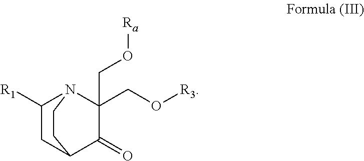

4. The compound according to claim 3 or an N-oxide thereof, or a pharmaceutically acceptable salt, solvate, polymorph, tautomer, stereoisomer, an isotopic form, or a prodrug thereof, wherein the compound is represented by Formula (IV):

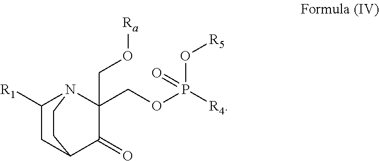

5. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (V):

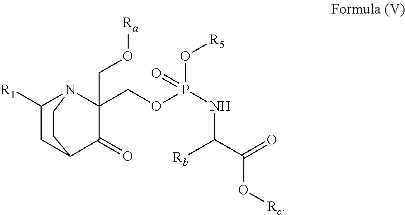

6. The compound according to claim 1 or an N-oxide thereof, or a pharmaceutically acceptable salt, solvate, polymorph, tautomer, stereoisomer, an isotopic form, or a prodrug thereof, wherein the compound is selected from the group consisting of:
(1R,2S,4S,6S)-2-(hydroxymethyl)-2-(methoxymethyl)-6-methylquinuclidin-3-one,
(1S,2S,4R,6S)-2-(hydroxymethyl)-2-(methoxymethyl)-6-methylquinuclidin-3-one,
(1R,2R,4S,6S)-2-(hydroxymethyl)-2-(methoxymethyl)-6-methylquinuclidin-3-one,
(1S,2R,4R,6S)-2-(hydroxymethyl)-2-(methoxymethyl)-6-methylquinuclidin-3-one,
(1R,2S,4S,6R)-2-(hydroxymethyl)-2-(methoxymethyl)-6-methylquinuclidin-3-one,
(1S,2S,4R,6R)-2-(hydroxymethyl)-2-(methoxymethyl)-6-methylquinuclidin-3-one,
(1R,2R,4S,6R)-2-(hydroxymethyl)-2-(methoxymethyl)-6-methylquinuclidin-3-one,
(1S,2R,4R,6R)-2-(hydroxymethyl)-2-(methoxymethyl)-6-methylquinuclidin-3-one,
(1R,2R,4S,6R)-6-ethyl-2-(hydroxymethyl)-2-(methoxymethyl)quinuclidin-3-one,
(1S,2R,4R,6R)-6-ethyl-2-(hydroxymethyl)-2-(methoxymethyl)quinuclidin-3-one,
(1R,2S,4S,6R)-6-ethyl-2-(hydroxymethyl)-2-(methoxymethyl)quinuclidin-3-one,
(1S,2R,4R,6S)-6-ethyl-2-(hydroxymethyl)-2-(methoxymethyl)quinuclidin-3-one,
(1R,2R,4S,6S)-6-ethyl-2-(hydroxymethyl)-2-(methoxymethyl)quinuclidin-3-one,
(1S,2S,4R,6R)-6-ethyl-2-(hydroxymethyl)-2-(methoxymethyl)quinuclidin-3-one,
(1S,2S,4R,6S)-6-ethyl-2-(hydroxymethyl)-2-(methoxymethyl)quinuclidin-3-one,
(1R,2S,4S,6S)-6-ethyl-2-(hydroxymethyl)-2-(methoxymethyl)quinuclidin-3-one,
(1R,2S,4S,6R)-2-(hydroxymethyl)-6-isopropyl-2-(methoxymethyl)quinuclidin-3-one,
(1S,2R,4R,6S)-2-(hydroxymethyl)-6-isopropyl-2-(methoxymethyl)quinuclidin-3-one,
(1R,2R,4S,6S)-2-(hydroxymethyl)-6-isopropyl-2-(methoxymethyl)quinuclidin-3-one,
(1S,2S,4R,6S)-2-(hydroxymethyl)-6-isopropyl-2-(methoxymethyl)quinuclidin-3-one,
(1R,2S,4S,6S)-2-(hydroxymethyl)-6-isopropyl-2-(methoxymethyl)quinuclidin-3-one,
(1S,2S,4R,6R)-2-(hydroxymethyl)-6-isopropyl-2-(methoxymethyl)quinuclidin-3-one,
(1R,2R,4S,6R)-2-(hydroxymethyl)-6-isopropyl-2-(methoxymethyl)quinuclidin-3-one,
(1S,2R,4R,6R)-2-(hydroxymethyl)-6-isopropyl-2-(methoxymethyl)quinuclidin-3-one,
(1S,2R,4R,6S)-2-(hydroxymethyl)-6-isobutyl-2-(methoxymethyl)quinuclidin-3-one,
(1S,2S,4R,6S)-2-(hydroxymethyl)-6-isobutyl-2-(methoxymethyl)quinuclidin-3-one,
(1R,2R,4S,6R)-2-(hydroxymethyl)-6-isobutyl-2-(methoxymethyl)quinuclidin-3-one,
(1R,2S,4S,6R)-2-(hydroxymethyl)-6-isobutyl-2-(methoxymethyl)quinuclidin-3-one,
(1S,2R,4R,6R)-2-(hydroxymethyl)-6-isobutyl-2-(methoxymethyl)quinuclidin-3-one,
(1S,2S,4R,6R)-2-(hydroxymethyl)-6-isobutyl-2-(methoxymethyl)quinuclidin-3-one,
(1R,2R,4S,6S)-2-(hydroxymethyl)-6-isobutyl-2-(methoxymethyl)quinuclidin-3-one,
(1R,2S,4S,6S)-2-(hydroxymethyl)-6-isobutyl-2-(methoxymethyl)quinuclidin-3-one
(1S,2R,4R,6R)-2-(hydroxymethyl)-2-(methoxymethyl)-6-neopentylquinuclidin-3-one,
(1S,2S,4R,6R)-2-(hydroxymethyl)-2-(methoxymethyl)-6-neopentylquinuclidin-3-one,
(1R,2R,4S,6S)-2-(hydroxymethyl)-2-(methoxymethyl)-6-neopentylquinuclidin-3-one,
(1R,2S,4S,6S)-2-(hydroxymethyl)-2-(methoxymethyl)-6-neopentylquinuclidin-3-one,
(1S,2R,4R,6S)-2-(hydroxymethyl)-2-(methoxymethyl)-6-neopentylquinuclidin-3-one
(1S,2R,4R,6S)-2-(hydroxymethyl)-2-(methoxymethyl)-6-neopentylquinuclidin-3-one,
(1R,2R,4S,6R)-2-(hydroxymethyl)-2-(methoxymethyl)-6-neopentylquinuclidin-3-one,
(1R,2S,4S,6R)-2-(hydroxymethyl)-2-(methoxymethyl)-6-neopentylquinuclidin-3-one,
(1R,2R,4S,6R)-2-(hydroxymethyl)-2-(methoxymethyl)-6-methylquinuclidin-3-one,
(1R,2R,4S,6R)-2-(hydroxymethyl)-6-methyl-2-((trifluoromethoxy)methyl)quinuclidin-3-one,
(1R,2R,4S,6R)-2-(hydroxymethyl)-2-((methoxy-d3)methyl)-6-methylquinuclidin-3-one,
((1R,2S,4S,6R)-2-(methoxymethyl)-6-methyl-3-oxoquinuclidin-2-yl)methyl acetate,
((1R,2S,4S,6R)-2-(methoxymethyl)-6-methyl-3-oxoquinuclidin-2-yl)methyl isobutyrate,
((1R,2S,4S,6R)-2-(methoxymethyl)-6-methyl-3-oxoquinuclidin-2-yl)methyl pivalate,
isopropyl (isopropoxy(((1R,2S,4S,6R)-2-(methoxymethyl)-6-methyl-3-oxoquinuclidin-2-yl)methoxy)phosphoryl)-L-phenylalaninate,
benzyl (isopropoxy(((1R,2S,4S,6R)-2-(methoxymethyl)-6-methyl-3-oxoquinuclidin-2-yl)methoxy)phosphoryl)-L-valinate,
isopropyl ((((1R,2S,4S,6R)-2-(methoxymethyl)-6-methyl-3-oxoquinuclidin-2-yl)methoxy)((S)-2-methyl-1-(propionyloxy)propoxy)phosphoryl)-L-phenylalaninate,
isopropyl ((((1R,2S,4S,6R)-2-(methoxymethyl)-6-methyl-3-oxoquinuclidin-2-yl)methoxy)(phenoxy)phosphoryl)-L-phenylalaninate,
benzyl ((((1R,2S,4S,6R)-2-(methoxymethyl)-6-methyl-3-oxoquinuclidin-2-yl)methoxy)(phenoxy)phosphoryl)-L-valinate,
benzyl ((((1R,2S,4S,6R)-2-(methoxymethyl)-6-methyl-3-oxoquinuclidin-2-yl)methoxy)(phenoxy)phosphoryl)-L-alaninate,
isopropyl ((((1R,2S,4S,6R)-2-(methoxymethyl)-6-methyl-3-oxoquinuclidin-2-yl)methoxy)(phenoxy)phosphoryl)-L-valinate, and
isopropyl ((((1R,2S,4S,6R)-2-(methoxymethyl)-6-methyl-3-oxoquinuclidin-2-yl)methoxy)(phenoxy)phosphoryl)-L-alaninate.

7. A pharmaceutical composition comprising a compound of Formula (I) or an N-oxide thereof of any one of claims 1-6, or a pharmaceutically acceptable salt, solvate, polymorph, tautomer, stereoisomer, an isotopic form, or a prodrug of said compound of Formula (I) or an N-oxide thereof, and a pharmaceutically acceptable diluent or carrier.

8. A method of treating a neoplastic disease, autoimmune disease, and inflammatory disorder, comprising administering to a subject in need thereof an effective amount of a compound of Formula (I) or an N-oxide thereof of any one of claims 1-6, or a pharmaceutically acceptable salt, solvate, polymorph, tautomer, stereoisomer, an isotopic form, or a prodrug of said compound of Formula (I) or an N-oxide thereof.

9. A compound of Formula (B), or an N-oxide thereof, or a pharmaceutically acceptable salt, solvate, polymorph, tautomer, stereoisomer, an isotopic form, or a prodrug of said compound of Formula (B) or N-oxide thereof: 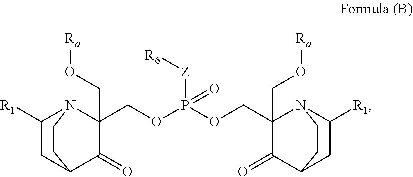
wherein
R₁ is H, D, alkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, alkenyl, alkynyl, cycloalkyl, cycloalkenyl, heterocycloalkyl, spiroheterocycloalkyl, heterocycloalkenyl, aryl, heteroaryl is optionally substituted with one or more R_{d};
Z is absent, O, or N(Rₐ);
R₆ is alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, cyano, —ORₐ, —SRₐ, -alkyl-Rₐ, —NH—(CHRₐ)COOR_{b}, —C(O)Rₐ, —S(O)Rₐ, —SO₂Rₐ, —C(O)ORₐ, —OC(O)Rₐ, —NHR_{b}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, C(O)NHOH, C(O)OH, —C(O)O-alkyl, —C(O)O-aryl, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ; and
Rₑ is H, D, alkyl, spiroalkyl, alkenyl, alkynyl, halo, cyano, amine, nitro, hydroxy, ═O, C(O)NHOH, —C(O)O-alkyl, —C(O)O-aryl, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl.

10. The compound according to claim 9 or an N-oxide thereof, or a pharmaceutically acceptable salt, solvate, polymorph, tautomer, stereoisomer, an isotopic form, or a prodrug thereof, wherein the compound is selected from the group consisting of:
((1R,2S,4S,6R)-2-(methoxymethyl)-6-methyl-3-oxoquinuclidin-2-yl)methyl (((2S,6R)-2-(methoxymethyl)-6-methyl-3-oxoquinuclidin-2-yl)methyl) methylphosphonate,
((1R,2S,4S,6R)-2-(methoxymethyl)-6-methyl-3-oxoquinuclidin-2-yl)methyl (((2S,6R)-2-(methoxymethyl)-6-methyl-3-oxoquinuclidin-2-yl)methyl) phenyl phosphate, and
isopropyl ((((1R,2S,4S,6R)-2-(methoxymethyl)-6-methyl-3-oxoquinuclidin-2-yl)methoxy)(((2S,6R)-2-(methoxymethyl)-6-methyl-3-oxoquinuclidin-2-yl)methoxy)phosphoryl)-L-phenylalaninate.

11. A pharmaceutical composition comprising a compound of Formula (B) or an N-oxide thereof of claim 9 or 10, or a pharmaceutically acceptable salt, solvate, polymorph, tautomer, stereoisomer, an isotopic form, or a prodrug of said compound of Formula (B) or an N-oxide thereof, and a pharmaceutically acceptable diluent or carrier.

12. A method of treating a neoplastic disease, autoimmune disease, and inflammatory disorder, comprising administering to a subject in need thereof an effective amount of a compound of Formula (B) or an N-oxide thereof of claim 9 or 10, or a pharmaceutically acceptable salt, solvate, polymorph, tautomer, stereoisomer, an isotopic form, or a prodrug of said compound of Formula (B) or an N-oxide thereof.

13. The method of claim 8 or 12, wherein the neoplastic disease is characterized by a mutant p53.

14. The method of claim 13, wherein the compound of Formula (I) or (B); the N-oxide thereof; or the pharmaceutically acceptable salt, solvate, polymorph, tautomer, stereoisomer, isotopic form, or prodrug thereof, restores biological function to the mutant p53.
